(19) United States
(12) Patent Application Publication
(10) Pub. No.: US 2025/0000908 A1
(43) Pub. Date: Jan. 2, 2025

(54) METHODS FOR PREPARING AND ADMINISTERING CRYOPRESERVED PLATELETS AND PLATELET DERIVATIVES LOADED WITH ANTI-CANCER DRUGS

(71) Applicant: Cellphire, Inc., Rockville, MD (US)

(72) Inventors: Keith Andrew MOSKOWITZ, Westfield, IN (US); Fengqian CHEN, Rockville, MD (US)

(73) Assignee: Cellphire, Inc., Rockville, MD (US)

(21) Appl. No.: 18/737,851

(22) Filed: Jun. 7, 2024

Related U.S. Application Data
(60) Provisional application No. 63/507,090, filed on Jun. 8, 2023.

(51) Int. Cl. A61K 35/19; A61K 31/704; A61K 47/10; A61K 47/20; A61K 47/26; A61P 35/00

(52) U.S. Cl. CPC A61K 35/19; A61K 31/704; A61K 47/10; A61K 47/20; A61K 47/26; A61P 35/00

(57) ABSTRACT

In some embodiments, provided herein is a composition comprising cryopreserved anti-cancer drug-loaded platelet composition, and a process for preparing the same, in illustrative embodiments, cryopreserved anti-cancer drug-loaded platelet composition can be stored at a temperature in a range of −10° C. to −30° C. for at least 1 month. The anti-cancer drug-loaded platelets in the composition can be platelet derivatives. In some embodiments provided herein are methods of preparing anti-cancer drug loaded platelet derivatives, or cryopreserved anti-cancer drug-loaded platelets, comprising: incubating platelets with an anti-cancer drug, and with a loading buffer comprising a monosaccharide and/or a disaccharide, to form anti-cancer drug-loaded platelets, and lyophilizing or freezing the anti-cancer drug-loaded platelets, to form anti-cancer drug loaded platelet derivatives, or cryopreserved anti-cancer drug-loaded platelets, respectively. In some embodiments, provided herein are methods of administering anti-cancer drug loaded platelet derivatives and cryopreserved platelets.

### CROSS-REFERENCE TO RELATED APPLICATIONS

This application claims priority to U.S. Provisional Application Ser. No. 63/507,090, filed on Jun. 8, 2023, and the content of the U.S. Provisional application listed above is incorporated herein by reference in its entirety.

### TECHNICAL FIELD

The present disclosure relates to cryopreserved platelets, and platelet derivatives, such as freeze-dried platelet derivatives, as biological carriers of drugs, such as, but not limited to, anti-cancer drugs.

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

There are a few studies available to suggest that platelets can be used as a delivery system for delivering cargo molecules. However, there is a long-felt need to provide a reliable source of targeted delivery of therapeutic agents, for example, a biologic comprising anti-cancer drugs, that are easily manufacturable, stable at a temperature that is easy to maintain in different parts of the world and amenable to long-term storage and transportation. Cryopreservation is a known method of preserving platelets, however, it is known that stringent freezing temperature conditions are required for storing cryopreserved platelets, for example, ultra-freezers are required to maintain temperatures at ≤−65° C. in order to have a functional platelet product. Accordingly, there is a long-felt need in the art to have a cryopreserved platelet composition that is effective in delivering therapeutic agents like anti-cancer drugs, and is readily manufacturable and that can be stored under conditions that do not require highly specialized equipment, for example that can be stored in standard −20° C. freezers for months or even years.

### SUMMARY OF THE INVENTION

To address the long-felt needs mentioned in the Background section, the present disclosure provides aspects and embodiments that include anti-cancer drug-loaded platelet derivatives, or cryopreserved anti-cancer drug-loaded platelets, methods for preparing anti-cancer drug-loaded platelet derivatives or cryopreserved anti-cancer drug-loaded platelets, and methods for administering an anti-cancer drug to a subject. In an aspect, cryopreserved anti-cancer drug-loaded platelets herein are capable of being stored in a −20° C. freezer, for example at a temperature in a range of −10° C. to −30° C., for at least 1 month, 3, or 6 months, and are capable of retaining at least 30% of the anti-cancer drug upon thawing.

Loading platelets with drugs, for example anti-cancer drugs, can allow targeted delivery of the drugs to sites of interest, such as tumors and cancer cells therein. Further, drug-loaded platelets can be lyophilized or cryopreserved to allow for long-term storage. In some embodiments the loading of a drug in the platelets mitigates systemic side effects associated with the drug and lowers the threshold of therapeutic dose necessary by facilitating targeted treatment at site of interest.

In some aspects, provided herein is a process for preparing a cryopreserved anti-cancer drug-loaded platelet composition that can be stored at a temperature higher than −65° C., for example, at a temperature in the range of −10° C. to −30° C. for a time period of at least 1 month, 3 months, 12 months, or until the cryopreserved anti-cancer drug-loaded platelets are required for treating a subject in need thereof. Also provided is a frozen platelet composition comprising anti-cancer drug-loaded cryopreserved platelets, which is capable of being stored at a temperature higher than −65° C., for example at a temperature in the range of −10° C. to −30° C. for a time period of at least 1 month, 3 months, 12 months, or until the cryopreserved platelets are required for treating a subject in need thereof.

Accordingly, provided herein in one aspect is a process for preparing a cryopreserved anti-cancer drug-loaded platelet composition comprising anti-cancer drug-loaded cryopreserved platelets, said process comprising:
a) incubating platelets with an anti-cancer drug in the presence of a loading buffer comprising a monosaccharide and/or a disaccharide at a temperature in the range of 18-42° C. for a time period in the range of 20 minutes to 12 hours, 20 minutes to 6 hours, or 20 minutes to 3 hours to obtain a population of anti-cancer drug-loaded platelets;
b) freezing the population of anti-cancer drug-loaded platelets in a cryopreservation medium at a temperature of equal to or less than −50° C. to form an initial frozen anti-cancer drug-loaded platelet composition; and
c) storing the initial frozen anti-cancer drug-loaded platelet composition at a temperature in the range of −10° C. to −30° C. for at least 10 days to form the anti-cancer drug-loaded cryopreserved platelet composition.

Accordingly, provided herein in one aspect is composition comprising frozen anti-cancer drug-loaded platelets in a cryopreservation medium in a frozen state, wherein the composition is capable of yielding the following recited properties after storage for 6 months, upon thawing:
a) is in a liquid state without requiring the addition of a liquid to achieve such liquid state;
b) exhibits a platelet count of at least 1.0×10¹¹/35 ml of the composition;
c) yields a single peak that corresponds to a compromised membrane peak in a membrane integrity assay;
d) exhibits a CD61-positive-microparticle content of less than 50% of the CD61 positive particles in the composition, and
e) retains at least 40% of the anti-cancer drug. In some embodiments, the composition upon thawing generates thrombin in an in vitro thrombin generation assay.

Provided herein in some aspects, is a method for administering an anti-cancer drug to a subject, comprising:
administering a therapeutically effective dose of anti-cancer drug-loaded platelet derivatives, or cryopreserved anti-cancer drug-loaded platelets, wherein the subject has cancer. In illustrative embodiments the subject is other than a contraindicated person, wherein the contraindicated person has a stent, has or had a heart condition, has or had a diagnosed blood clot, and/or has an effective amount of an anti-platelet agent in their bloodstream.

Provided herein in some aspects, is a method for administering an anti-cancer drug to a subject, comprising:
a) preparing anti-cancer drug loaded platelet derivatives, or cryopreserved anti-cancer drug-loaded platelets by loading the anti-cancer drug into platelets and lyophilizing or freezing the platelets to prepare the anti-cancer drug loaded platelet derivatives, or the cryopreserved anti-cancer drug-loaded platelets, respectively, wherein the anti-cancer drug loaded platelet derivatives, or the cryopreserved anti-cancer drug-loaded platelets comprise the anti-cancer drug;
b) storing the anti-cancer drug loaded freeze-dried platelet derivatives, or the cryopreserved anti-cancer drug-loaded platelets for at least 2, 3, 4, 5, 6, 7, or 8 days; and
c) administering a therapeutically effective dose of the anti-cancer drug-loaded platelet derivatives or the cryopreserved anti-cancer drug-loaded platelets to the subject.

Not to be limited by theory, drug-loaded platelets, cryopreserved drug-loaded platelets, drug-loaded platelet derivatives, or drug-loaded thrombosomes provided herein can shield the drug from exposure in circulation, thereby reducing or eliminating systemic toxicity (e.g. cardiotoxicity) associated with the drug. Drug-loaded platelets, cryopreserved drug-loaded platelets, drug-loaded platelet derivatives, or drug-loaded thrombosomes can also protect the drug from metabolic degradation or inactivation. Drug delivery with drug-loaded platelets, cryopreserved drug-loaded platelets, drug-loaded platelet derivatives, or drug-loaded thrombosomes can therefore be advantageous in treatment of diseases such as cancer, wherein the drug comprises an anti-cancer agent or an anti-cancer drug. Anti-cancer drug-loaded platelets, cryopreserved anti-cancer drug loaded platelets, anti-cancer drug-loaded platelet derivatives, or anti-cancer drug-loaded thrombosomes can facilitate targeting of cancer cells while mitigating systemic side effects. Therefore, compositions comprising or process herein producing anti-cancer drug-loaded platelets, cryopreserved anti-cancer drug-loaded platelets, anti-cancer drug-loaded platelet derivatives, or anti-cancer drug-loaded thrombosomes can be used in any therapeutic setting in which expedited healing process is required or advantageous. Potential applications include, for example, targeted depletion of cancer cells with chemotherapy drugs.

Accordingly, in some embodiments, provided herein is a method of treating a disease, such as cancer as disclosed herein, comprising administering anti-cancer drug-loaded platelets, cryopreserved anti-cancer drug-loaded platelets, anti-cancer drug-loaded platelet derivatives, or anti-cancer drug-loaded thrombosomes as disclosed herein. Accordingly, in some embodiments, provided herein is a method of treating cancer, comprising administering cold stored, room temperature stored, cryopreserved thawed, rehydrated, and/or lyophilized anti-cancer drug-loaded platelets, anti-cancer drug-loaded platelet derivatives, or anti-cancer drug-loaded thrombosomes as disclosed herein.

Further details regarding aspects and embodiments of the present disclosure are provided throughout this patent application. Sections and section headers are for ease of reading and are not intended to limit combinations of disclosure, such as methods, compositions, and kits or functional elements therein across sections. Further details regarding aspects and embodiments of the present disclosure are provided throughout this patent application. Sections and section headers are for ease of reading and are not intended to limit combinations of disclosure, such as methods, compositions, or other functional elements therein across sections.

### BRIEF DESCRIPTION OF THE DRAWINGS

FIG. 1 shows a non-limiting flow chart of an exemplary process for preparing a cryopreserved anti-cancer drug-loaded platelet composition that is capable of storing at a temperature in the range of −10° C. to −30° C.

FIG. 2 shows a comparison of doxorubicin in doxorubicin-loaded platelet derivatives (Post-lyo-FPH) with doxorubicin-loaded platelets before lyophilization (Pre-lyo-FPH), and a comparison of doxorubicin in thawed cryopreserved doxorubicin-loaded platelets (Post-cryo-CPP) with doxorubicin-loaded platelets before cryopreservation (Pre-cryo-CPP).

FIG. 3A shows stability of cryopreserved doxorubicin-loaded platelets (DOX-CPP), and doxorubicin-loaded platelet derivatives (DOX-FPD) in terms of number of platelets or platelet derivatives/μl over a period of 14 days.

FIG. 3B shows stability of cryopreserved doxorubicin-loaded platelets (DOX-CPP), and doxorubicin-loaded platelet derivatives (DOX-FPD) in terms of doxorubicin femtograms (fg)/cryopreserved platelets or platelet derivatives over a period of 18 days.

FIG. 4 shows the clot formation time for cryopreserved doxorubicin-loaded platelets (DOX-CPP), unloaded CPP, doxorubicin-loaded platelet derivatives (DOX-FPH), and unloaded FPH.

FIG. 5 shows the surface interaction between the HepG2 cells and cryopreserved platelets (CPP) or platelet derivatives (FPH).

FIG. 6 shows the intracellular uptake of DOX-CPP, DOX-FPH, Doxosome, and free DOX in HepG2 cells.

FIG. 7 shows fluorescent images of doxorubicin released from DOX-CPP inside the HepG2 cells.

FIG. 8A shows the interaction between cryopreserved platelets (CPP), and HepG2 cells in the absence (Control; Cl), and in the presence of inhibitors, Wortmannin (Wo), Tirofiban (Ti), and Podoplanin (Po).

FIG. 8B shows the interaction between platelet derivatives (FPH), and HepG2 cells in the absence (Control; Cl), and in the presence of inhibitors, Wortmannin (Wo), Tirofiban (Ti), and Podoplanin (Po).

FIG. 9A shows the effect of concentration (plt/μl) of DOX-CPP, DOX-FPH, unloaded CPP, and unloaded FPH on the viability of HepG2 cells.

FIG. 9B shows the effect of DOX concentration (μM) in DOX-CPP, DOX-FPH, unloaded CPP, and unloaded FPH on the viability of HepG2 cells.

FIG. 10A shows the distribution of DiR labeled DOX-CPP and DiR labeled Doxosomes in mice during the different time courses.

FIG. 10B shows the accumulation of DiR labeled DOX-CPP and DiR labeled Doxosomes in different organs in mice.

FIG. 11A shows the accumulation of an undiluted dose (2.18 mg/kg) of DOX-CPP and Doxosomes in mice for time points, 6 h, 12 h, 24 h, and 48 h post administration.

FIG. 11B shows the accumulation of 1:4 diluted dose (0.54 mg/kg) of DOX-CPP and Doxosomes in mice for time points, 6 h, 12 h, 24 h, and 48 h post administration.

FIG. 11C shows the accumulation of 1:10 diluted dose (0.218 mg/kg) of DOX-CPP and Doxosomes in mice for time points, 6 h, 12 h, 24 h, and 48 h post administration.

FIG. 12A shows an overall decrease in tumor burden (BLI) in mice for DOX-CPP and Doxosomes in a dose-dependent manner at a 12-hour time-point post administration.

FIG. 12B shows an overall decrease in tumor burden (BLI) in mice for DOX-CPP and Doxosomes in a dose-dependent manner at a 24-hour time-point post administration.

FIG. 12C shows an overall decrease in tumor burden (BLI) in mice for DOX-CPP and Doxosomes in a dose-dependent manner at a 48-hour time-point post administration.

FIG. 13A shows the tumor weight in mice for all three doses of DOX-CPP and Doxosomes at different time points.

FIG. 13B shows the tumor weight in mice as a function of time for the undiluted dose (2.18 mg/kg) of DOX-CPP and Doxosomes.

FIG. 14A shows the accumulation of DOX-CPP and Doxosomes for the three dosages at the tumor site in mice at 24 days.

FIG. 14B shows the tumor weight at 24 days post administration of the three dosages each of DOX-CPP and Doxosomes.

### DEFINITIONS

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein, the term “fresh platelets” has its ordinary meaning in the art.

As used herein, “cryopreserved platelets” are frozen platelets that when thawed are in a liquid state regardless of whether any liquid is added to the frozen platelets after thawing. Accordingly, cryopreserved platelets are not fresh platelets and they are not freeze-dried platelet derivatives. During processing cryopreserved platelets are not dried. The term “cryopreserved platelets” does not imply any minimum length of time such platelets are present in a frozen state. However, cryopreserved platelets are typically stored for at least 24 hours in a frozen state. Cryopreserved platelets are typically suspended in a cryoprotectant in a frozen state, until thawing before use. In some embodiments herein, cryopreserved platelets are stored for a period of at least 1, 2, 3, 4, 5, 6, 9, or 12 months at a temperature of −20° C.

As used herein, “hemostatic properties” include the following properties: (a) the ability to generate thrombin in a thrombin generation assay, for example in the presence of tissue factor and phospholipids; (b) the ability to occlude a collagen-coated microchannel in vitro, for example under conditions in which fresh platelets can occlude a collagen-coated microchannel in vitro; (c) the capability of thrombin-induced trapping in the presence of thrombin. As demonstrated in Examples herein, platelet derivatives, such as a freeze-dried platelet derivatives (e.g., thrombosomes) herein, in illustrative embodiments are hemostats, and thus have one, two, or all of the aforementioned hemostatic properties.

As used herein, “platelet derivatives” are particles that have some characteristics of fresh platelets but are surrounded by a compromised plasma membrane (i.e., lack an integrated membrane around them), and as such include pores that are larger than pores found in living platelets. Thus, in illustrative embodiments, platelet derivatives herein exhibit an increased permeability to IgG antibodies. In illustrative embodiments, platelet derivatives, or aggregates thereof found in platelet compositions, are at least 0.5 μm or between 0.5 μm and 25 μm in diameter as determined by dynamic light scattering. Thus, such subsets of platelet-derivative particles are distinguishable from platelet-derivative microparticles, which have a diameter of less than 0.5 μm. Dry platelet derivatives are typically present in a dried substance that includes other components present along with the platelet derivatives when they were dried. Furthermore, a dry platelet derivative(s) with at least one hemostatic property can be referred to as a dry platelet derivative hemostat(s) (PDH).

As used herein “platelet derivatives” in the context of cryopreserved platelets, or a composition comprising frozen platelets and/or platelet derivatives implies particles that, unlike fresh platelets, do not have intact cell membranes, for example, as demonstrated by a Calcein AM membrane integrity assay. Accordingly, in some embodiments, a composition provided herein comprises platelet derivatives, and such platelet derivatives are not freeze-dried platelet derivatives.

In illustrative embodiments, platelet derivatives herein have a reduced ability to, or are unable to transduce signals from the external environment into a response inside the particle that are typically transduced in living platelets. However, platelet derivatives herein (e.g., thrombosomes) can retain some metabolic activity, for example, as evidenced by lactate dehydrogenase (LDH) activity and/or esterase activity.

As used herein, “particle size” refers to the diameter of a particle, unless indicated otherwise. In some embodiments of any of the aspects and embodiments herein that include a platelet derivative composition in a powdered form, the size of the particles is determined after rehydrating the platelet derivative composition with an appropriate solution. In some embodiments, the amount of solution for rehydrating a platelet derivative composition is equal to the amount of buffer or preparation agent present at the step of freeze-drying. The particle size distribution and microparticle content of a composition can be measured by any appropriate method, for example, by flow cytometry using sizing standards, or in illustrative embodiments by dynamic light scattering (DLS). As used herein, a content (e.g., ratio or percent) of microparticles in illustrative embodiments refers to the microparticle content based on the scattering intensity of all particles from about 1 nm to about 60,000 nm in radius in the composition. In some cases, the viscosity of a sample used for DLS can be at about 1.060 cP (or adjusted to be so), as this is the approximate viscosity of plasma. It will be understood that the measured size of particles can vary depending on the technology used to perform the measurement. Particle sizes provided herein are typically as determined by DLS unless the context indicates otherwise. For example, if a percent surface marker content of a composition that includes particles, is recited as a percent of particles or platelet derivatives within a certain size range, flow cytometry is typically used to measure both biomarker content and particle size. In some embodiments, the platelet derivative composition as per any aspects, or embodiments comprises a population of platelet derivatives greater than 0.5 μm, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, illustrative or target platelet derivatives have a diameter in the range of 0.5-2.5 μm using flow cytometry, or a diameter of 0.5-25 μm using DLS, and microparticles have a diameter less than 0.5 μm by either method.

As used herein, “thrombosomes” (sometimes also herein called “Tsomes” or “Ts”) are platelet derivatives that have been contacted with an incubating agent (e.g., any of the incubating agents described herein) and lyopreserved (e.g., freeze-dried). Thus, thrombosomes are illustrative or target freeze-dried platelet derivatives (FDPDs). Illustrative or target freeze-dried platelet derivative compositions herein (e.g. thrombosomes) typically have at least 1 hemostatic property, and thus can function as hemostatic agents and can be referred to hemostat(s) or hemostatic product(s). Illustrative or target FDPDs and compositions herein comprising the same that have at least 1 hemostatic property can also be referred to as freeze-dried platelet derived hemostat(s) or freeze-dried platelet hemostat(s) (both of which can be abbreviated FDPDH, FDPH or FPH). In some cases, illustrative or target FDPDs such as thrombosomes can be prepared from pooled platelets. Illustrative or target FDPDs such as thrombosomes can have a shelf life of 2-3 years in dry form at ambient temperature and can be rehydrated with sterile water within minutes (e.g. 1, 2, 3, 4, 5, 10 15, 20, 25, or 30 minutes) for immediate infusion. One example of thrombosomes are THROMBOSOMES® freeze-dried platelet derivatives (Cellphire Inc., Rockville, MD), which are in clinical trials for the treatment of acute hemorrhage in thrombocytopenia patients and are a product of Cellphire, Inc. In non-limiting illustrative embodiments, FDPD compositions, or illustrative or target freeze-dried platelet-derivative (i.e. “FDPD”) compositions herein, or anti-cancer drug-loaded platelet derivatives, such as those prepared according to Example 1 herein, are compositions that include a population of platelet derivatives having a reduced propensity to aggregate such that no more than 10% of the platelet derivatives in the population aggregate under aggregation conditions comprising an agonist but no platelets, and in illustrative embodiments, no divalent cations. Furthermore, such illustrative or target platelet derivatives typically have the ability to generate thrombin in an in vitro thrombin generation assay and/or have the ability to occlude a collagen-coated microchannel in vitro.

As used herein “anti-cancer drug-loaded platelets”, “cryopreserved anti-cancer drug-loaded platelets” “anti-cancer drug-loaded platelet derivatives” can be understood to include the platelets or platelet derivatives that are associated with the anti-cancer drug in any form. The platelets or platelet derivatives can have the anti-cancer drug inside the platelets or platelet derivatives. The “anti-cancer drug-loaded platelets”, “cryopreserved anti-cancer drug-loaded platelets”, or “anti-cancer drug-loaded platelet derivatives” can contain, comprise, and/or otherwise be associated with, or bound to the anti-cancer drug. The anti-cancer drug-loaded platelets provided herein can be classified as biologics, and can be loaded with anti-cancer drugs of various classifications (e.g., small molecules, targeted therapies, biologics), as provided herein.

It is to be understood that the terminology used herein is for the purpose of describing particular aspects and embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment. As used herein, the symbol “<” means less than or in the context of temperatures, can mean below a recited temperature. As used herein, the symbol “/” in the context of temperatures, can mean to include a range of temperature, for example −20° C.+/−2° C. would mean a temperature from −18° C. to −22° C. As used herein, “about” or “consisting essentially of” mean±10% of the indicated range, value, or structure, unless otherwise indicated. As used herein, the terms “include” and “comprise” are open ended and are used synonymously. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

While the embodiments of the present disclosure are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

It is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### DETAILED DESCRIPTION

The present disclosure addresses many long-felt needs and long-standing problems in the art, such as, but not limited to those mentioned in the Background section herein. To overcome the above-mentioned and additional problems in the art, the present disclosure provides aspects and embodiments that include biologic therapeutic agents, for example, cryopreserved anti-cancer drug-loaded platelets for treating cancer that are capable of being stored at a temperature in a range of −10° C. to −30° C. (e.g., in a −20 freezer) for at least 1 month, 3, 6 months, or longer. In some embodiments, biologic therapeutic agents herein can deliver anti-cancer drugs in a manner superior to that of the anti-cancer drugs in liposomes. Surprisingly, methods disclosed herein that involve a transition from an initial freezing temperature to a warmer frozen storing temperature (e.g., from a freezing temperature of equal to or less than −50° C. to a storage temperature of −10° C. to −30° C.), provide such cryopreserved anti-cancer drug-loaded platelets that can be stored for at least 1 month, 3, 6 months, or longer. Such methods, thus provide an advantage in terms of simplifying the storage conditions and delivering a platform for administering anti-cancer drugs to subjects in need thereof.

In some aspects and embodiments, provided herein are anti-cancer drug-loaded platelet derivatives, cryopreserved anti-cancer drug-loaded platelets, methods for preparing anti-cancer drug-loaded platelet derivatives or cryopreserved anti-cancer drug-loaded platelets, and methods for administering an anti-cancer drug to a subject. In some aspects and embodiments, cryopreserved anti-cancer drug-loaded platelets provided herein are capable of being stored at a temperature in a range of −10° C. to −30° C. for at least 1 month, 3 months, 6 months, or longer, and are capable of retaining at least 30% of the anti-cancer drug upon thawing. Aspects and embodiments herein provide processes for preparing such cryopreserved anti-cancer drug-loaded platelets.

In some aspects and embodiments, anti-cancer drug-loaded platelet derivatives provided herein are capable of being stored at a temperature in a range of 20° C. to 30° C. for at least 1 year, and are capable of retaining at least 30% of the anti-cancer drug upon rehydrating.

Accordingly, provided herein, in some aspects, is a process comprising a step of incubating platelets with an anti-cancer drug in the presence of a loading buffer comprising a monosaccharide and/or a disaccharide at a temperature in the range of 18-42° C. for a time period in the range of 20 minutes to 12 hours, to obtain a population of anti-cancer drug-loaded platelets, the population of anti-cancer drug-loaded platelets are subjected to initial freezing at a temperature (i.e., initial temperature) less than or equal to −50° C., −60° C., −65° C., −70° C., −80° C., −85° C., or −90° C., or in the range of −50° C. to −85° C., or −60° C. to −85° C. to form an initial frozen anti-cancer drug-loaded platelet composition, followed by storing the initial frozen anti-cancer drug-loaded platelet composition in a frozen state at a temperature (i.e., storage temperature) equal to or greater than −30° C., but less than 0° C., to form a cryopreserved anti-cancer drug-loaded platelet composition. Surprisingly, it was found that the anti-cancer drug-loaded cryopreserved platelets formed by the process as disclosed herein have the property of being stable and retain hemostatic abilities when stored at higher temperatures as compared to that required for storing conventional cryopreserved platelets. For example, the anti-cancer drug-loaded cryopreserved platelets prepared as per a process disclosed herein can be stored at a temperature of about −30° C., −25° C., −20° C., −15° C., −10° C., or −5° C. or at a temperature in the range of −30° C. to −5° C., or −30° C. to −5° C. for at least 1 month up to 2, 3, 4, 5, 6, 7, 8, 9, 10, 11, or 12 months, or for at least 2, 3, 4, 5, 6, 7, 8, 9, 10, 11, or 12 months, or for at least, or up to 1 year, 2, 3, 4, 5, or 6 years, or for between 6 months and 1 year, 2, 3, 4, 5, or 6 years.

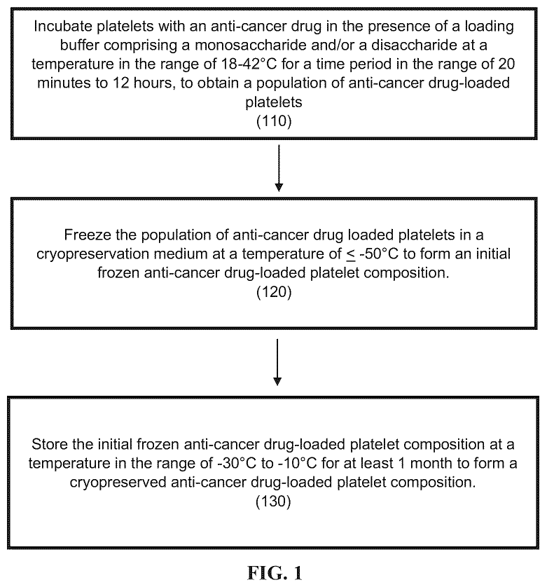

Accordingly, as illustrated in FIG. 1, provided in some aspects, are processes for preparing a cryopreserved anti-cancer drug-loaded platelet composition. Exemplary steps of such aspects are shown in boxes in FIG. 1. Such methods, in non-limiting examples, can include the following steps:
a) incubating platelets with an anti-cancer drug in the presence of a loading buffer comprising a monosaccharide and/or a disaccharide at a temperature in the range of 18-42° C. for a time period in the range of 20 minutes to 12 hours, to obtain a population of anti-cancer drug-loaded platelets;
b) freezing the population of anti-cancer drug-loaded platelets in a cryopreservation medium at a temperature of equal to or less than-50° C. to form an initial frozen anti-cancer drug-loaded platelet composition; and
c) storing the initial frozen anti-cancer drug-loaded platelet composition at a temperature in the range of −10° C. to −30° C. for at least 10 days to form the anti-cancer drug-loaded cryopreserved platelet composition.

Provided herein, in one aspect is a composition comprising frozen anti-cancer drug-loaded platelets in a cryopreservation medium in a frozen state, wherein the composition is capable of yielding the following recited properties after storage for 6 months, upon thawing:
a) is in a liquid state without requiring the addition of a liquid to achieve such liquid state;
b) exhibits a platelet count of at least 1.0×10¹¹/35 ml of the composition;
c) yields a single peak that corresponds to a compromised membrane peak in a membrane integrity assay;
d) exhibits a CD61-positive-microparticle content of less than 50% of the CD61 positive particles in the composition, and
e) generates thrombin in an in vitro thrombin generation assay.
f) retains at least 40% of the anti-cancer drug.

### Preparation of Cryopreserved Platelets Using a Transition in Freezing Temperatures

Provided herein in one aspect is a process for preparing cryopreserved anti-cancer drug-loaded platelets that include an initial freezing step comprising loading platelets with an anti-cancer drug, to form a population of anti-cancer drug-loaded platelets, followed by freezing the population of anti-cancer drug-loaded platelets in a cryopreservation medium, at a temperature of less than or equal to −50° C., −55° C., −60° C., in illustrative embodiments, less than or equal to −65° C., −70° C., −75° C., or −80° C., to form an initial frozen anti-cancer drug-loaded platelet composition, and a second step comprising storing the initial frozen anti-cancer drug-loaded platelet composition in a frozen state at a temperature of more than or equal to −40° C., −35° C., −30° C., −25° C., in illustrative embodiments, more than or equal to −20° C., −15° C., or −10° C., but less than 0° C. to form cryopreserved anti-cancer drug-loaded platelets, or cryopreserved anti-cancer drug-loaded platelet composition. In some embodiments, a composition that includes cryopreserved anti-cancer drug-loaded platelets obtained from a process using a transition in freezing temperatures from an initial temperature equal to or less than some initial target temperature set at a target initial temperature or temperature range that is no warmer than −50° C., and then stored after some period of time, at a temperature that is at a target storage temperature set at a target storage temperature or storage temperature range between about −10° C. to about-30° C., can be referred to as a transition temperature cryopreserved-product, or a transition temperature-cryopreserved composition, and such a process can be referred to as a transition temperature cryopreservation process. Also, for convenience to differentiate a transition temperature cryopreserved product from a cryopreserved product that does not involve such a transition in temperature in its preparation, in some embodiments, a cryopreserved product that is obtained by only freezing and storing at a target temperature at or below −50° C., −60° C., for example about −80° C., is referred to as a single temperature cryopreserved-product. A skilled artisan will understand that such single-temperature cryopreserved product can in fact, be subjected to a variation in temperatures, but such variation does not include a transition from an initial freezing temperature at or below −50° C. to a target storage temperature at −10° C. to −30° C. Surprisingly, cryopreserved platelets, or cryopreserved anti-cancer drug-loaded platelets obtained by a process including a transition in temperature as disclosed herein when stored at a temperature of equal to or higher than −30° C. but less than 0° C., or −5° C. upon thawing can exhibit hemostatic properties, and in illustrative embodiments are capable of delivering anti-cancer drug to a subject, thereby addressing a long-felt need in storage conditions of cryopreserved platelets.

In some embodiments, an initial frozen anti-cancer drug-loaded platelet composition can be stored at a freezing temperature of more than −40° C., −35° C., −30° C., −25° C., −20° C. In some embodiments, storing of an initial frozen anti-cancer drug-loaded platelet composition can be done for at least 30 minutes, 1 hour, 2, 3, 6, 8, 10, 12, 18, 24 hours, 2 days, 3, 5, 7, 15, 20, 25, days, 1 month, 2, 3, 4, 5, 6, 7, 8, 9, 10, 11, or 12 months, or 1 year, 2, 3, 4, 5, or 6 years. In some embodiments, storing of an initial frozen anti-cancer drug-loaded platelet composition can be done for a time period in the range of 1 month to 10 years, 1 month to 8 years, 1 month to 6 years, 1 month to 5 years, 1 month to 3 years, 1 month to 2 years, 1 month to 1 year, 6 months to 10 years, 1 year to 10 years, 2 years to 10 years, or 3 years to 10 years. In some cases, an initial frozen anti-cancer drug-loaded platelet composition can be stored at a temperature in the range of −40° C. to −10° C. until the cryopreserved anti-cancer drug-loaded platelets are used for treating a subject in need thereof, in illustrative embodiments, for administering to a subject having cancer.

Typically, in an initial freezing step, a cryopreservation medium having anti-cancer drug-loaded platelets, as disclosed herein, become frozen, and achieve the temperatures as disclosed therein to form an initial frozen anti-cancer drug-loaded platelet composition. For example, freezing a cryopreservation medium having anti-cancer drug-loaded platelets, as disclosed herein at a temperature in the range of −50° C. to −85° C. comprises subjecting the cryopreservation medium to the temperature range such that an initial frozen anti-cancer drug-loaded platelet composition is formed at the end of the step, and the temperature of the initial frozen anti-cancer drug-loaded platelet composition is in the range of −50° C. to −85° C. An initial freezing step can be performed for a time period until the cryopreservation medium having the anti-cancer drug-loaded platelets reaches a temperature less than or equal to −50° C., −55° C., −60° C., −65° C., −70° C., −75° C., or −80° C., and the time it takes for the cryopreservation medium to attain the temperature can depend on various factors, not limited to the volume of a cryo-vessel, dimensions of a cryo-vessel, volume of cryopreservation medium having the anti-cancer drug-loaded platelets, concentration of anti-cancer drug-loaded platelets in a cryo-vessel, and composition of a cryopreservation medium in a cryo-vessel. A cryopreservation medium that can be used in a process as disclosed herein can be a cryopreservation medium comprising a cryoprotectant. In illustrative embodiments, the cryoprotectant comprises dimethyl sulfoxide (DMSO). In other embodiments, the cryoprotectant can be any other cryoprotectant apart from DMSO. Other non-limiting examples of suitable cryoprotectants can include saccharides, such as monosaccharides and disaccharides, including sucrose, maltose, trehalose, glucose, mannose, dextrose, xylose, and a combination thereof. In some embodiments, a cryopreservation medium comprising DMSO as a cryoprotectant can have a concentration in the range of 0.001-10%, 0.5-7%, 1-8%, 2-8%, 3-8%, 4-8%, or 5-8%. In some embodiments, a cryopreservation medium can be a loading buffer comprising monosaccharides and/or disaccharides, and in some embodiments, monosaccharides can be selected from glucose, mannose, and dextrose, and disaccharides can be selected from sucrose, trehalose, maltose, and xylose. In some embodiments, cryopreservation medium can be the loading buffer that is used for incubating platelets with an anti-cancer drug in an earlier step. In other embodiments, cryopreservation medium can be a different composition as compared to the loading buffer that is used for incubating platelets with an anti-cancer drug in an earlier step. In some embodiments, an initial freezing step can be done for at least for at least 30 minutes, 1 hour, 2 hours, or 3 hours. For example, an initial freezing step can be done for a time period in the range of 30 minutes to 12 hours, 30 minutes to 10 hours, 30 minutes to 8 hours, 30 minutes to 6 hours, or 30 minutes to 4 hours. In some embodiments, an initial freezing step can be done for more than 12 hours, 2 days, 3 days, 1 week, 1 month, or 6 months. In some embodiments, the temperature during an initial freezing step can be in the range of −50° C. to −90° C., −50° C. to −85° C., −50° C. to −80° C., −50° C. to −75° C., −50° C. to −70° C., −55° C. to −90° C., −60° C. to −90° C., −60° C. to −85° C., −60° C. to −80° C., −60° C. to −75° C., or −65° C. to −75° C. In illustrative embodiments, the temperature during an initial freezing step can be −65° C.+/−5° C., −65° C.+/−4° C., −65° C.+/−3° C., −65° C.+/−2° C., or −65° C.+/−1° C. In other illustrative embodiments, the temperature during an initial freezing step can be −80° C.+/−5° C., −80° C.+/−4° C., −80° C.+/−3° C., −80° C.+/−2° C., or −80° C.+/−1° C. In some embodiments, the time-period during an initial freezing step can depend on the temperature that needs to be achieved. For example, an initial freezing step can comprise a temperature in the range of −60° C. to −80° C., for a time period in the range of 1 hour to 7 hours, 1 hour to 6 hours, 1 hour to 5 hours, 1 hour to 4 hours, or 1 hour to 2 hours. In some embodiments, an initial freezing step comprises placing a cryopreservation medium having anti-cancer drug-loaded platelets in a freezer set at a temperature in the range of −50° C. to −90° C., to form an initial frozen anti-cancer drug-loaded platelet composition.

In some embodiments, storing an initial frozen anti-cancer drug-loaded platelet composition comprises subjecting an initial frozen anti-cancer drug-loaded platelet composition to a temperature equal to or more than −30° C., −25° C., −20° C., −15° C., or −10° C. but less than −5° C. In illustrative embodiments, an initial frozen anti-cancer drug-loaded platelet composition is subjected to a temperature of −20° C.+/−5° C., −20° C.+/−4° C., −20° C.+/−3° C., −20° C.+/−2° C., −20° C.+/−1° C., or −20° C.+/−0.5° C. In some embodiments, storing an initial frozen anti-cancer drug-loaded platelet composition comprises storing in a freezer that is set at a temperature in a range of −10° C. to −40° C., −10° C. to −30° C., or −15° C. to −25° C. Typically, an initial frozen anti-cancer drug-loaded platelet composition when stored at a temperature as disclosed herein or when subjected to a temperature as disclosed herein reaches the intended temperature at the end of the step to form cryopreserved anti-cancer drug-loaded platelets, and after the step, the cryopreserved anti-cancer drug-loaded platelets is stored at the temperature disclosed herein for at least 7, 10, 15, 20, 25 days, 1 month, 2, 3, 4, 6, 8, 10, 12 months, 1 year, 2, 3, 4, 5, 6, 7, 8, 9, or 10 years, or until the cryopreserved anti-cancer drug-loaded platelets are used for administering to or treating a subject in need thereof. In some embodiments, storing an initial frozen anti-cancer drug-loaded platelet composition can comprise storing at a freezing temperature of equal to or higher than −30° C. for a time of at least 30 minutes, 45 minutes, 50 minutes, 60 minutes, 70 minutes, 80 minutes, 90 minutes, 2 hours, or 3 hours to form cryopreserved anti-cancer drug-loaded platelets. In some embodiments, a process disclosed herein can comprise subjecting an initial frozen anti-cancer drug-loaded platelet composition to a temperature equal to or higher than −30° C., in illustrative embodiments, in a range of −10° C. to −30° C. for a time until the temperature of the initial frozen anti-cancer drug-loaded platelet composition reaches the temperature of equal to or higher than −30° C., or in illustrative embodiments, in a range of −10° C. to −30° C. to form cryopreserved anti-cancer drug-loaded platelets. Typically, once the temperature of the initial frozen anti-cancer drug-loaded platelet composition reaches the temperature in a range of −10° C. to −30° C. to form a cryopreserved anti-cancer drug-loaded composition, the cryopreserved anti-cancer drug-loaded composition is be stored at a temperature in a range of −10° C. to −30° C. until the cryopreserved anti-cancer drug-loaded platelets are used for treating a subject in need thereof, or are used for administrating to a subject in need thereof.

### Composition Comprising Frozen/Cryopreserved Anti-Cancer Drug-Loaded Platelets

Compositions comprising frozen or cryopreserved anti-cancer drug-loaded platelets, or frozen or cryopreserved anti-cancer drug-loaded platelet derivatives provided herein, in some aspects and embodiments have one or more recited properties (which can also be referred to as recited attributes or recited characteristics). It will be understood that compositions that fall under such aspects or embodiments comprising one or more recited properties exhibit such one or more recited properties, but to fall under such aspects or embodiments that comprise such recited one or more properties does not require that a step is actually performed to demonstrate the one or more recited properties. However, a skilled artisan will understand that such one or more recited properties of a composition can be identified using a method that is set out by a recited property, or by performing a known method, to determine whether a test composition possesses such one or more recited properties. Frozen compositions herein that comprise anti-cancer drug-loaded platelets and/or anti-cancer drug-loaded platelet derivatives, upon thawing exhibit one or more of the following non-limiting recited properties: a) are capable of exhibiting a platelet count of at least 1.0×10¹¹ in 35 ml; b) have about 50% to about 99% of platelets and/or platelet-derived particles in the range of about 1 μm to about 2.5 μm or 5 μm; c) are in a liquid state without requiring the addition of a liquid to achieve such liquid state; d) yield a single peak that corresponds to a compromised membrane peak in a membrane integrity assay; e) exhibit a CD61-positive-microparticle content of less than 50% of the CD61 positive particles in the composition; f) retains at least 30%, 35%, 40%, 45%, 50%, 60%, or 70% of the anti-cancer drug; g) exhibit an ability to generate thrombin in an in vitro thrombin generation assay; h) are capable of inducing aggregation under in vitro aggregation conditions comprising an agonist; i) exhibit swirling upon visual observation of the composition; j) exhibit lack of aggregation upon visual observation of the composition; and/or k) exhibit lactadherin positivity in the range of 80-99.5%.

Provided herein in an aspect is a composition comprising frozen anti-cancer drug-loaded platelets, in an illustrative embodiment, frozen anti-cancer drug-loaded platelet derivatives, in a cryopreservation medium in a frozen state. In some embodiments, a composition comprising frozen anti-cancer drug-loaded platelets in a cryopreservation medium is a composition comprising cryopreserved anti-cancer drug-loaded platelets and/or cryopreserved anti-cancer drug-loaded platelet derivatives. In some embodiments, cryopreserved anti-cancer drug-loaded platelets herein can be cryopreserved anti-cancer drug-loaded platelet derivatives. Typically, a composition comprising frozen anti-cancer drug-loaded platelets upon thawing is in a liquid state without the addition of a liquid, such as water or a buffer. Without being bound by any theory, since the process of cryopreservation does not include the step of drying, the anti-cancer drug-loaded platelets in a cryopreservation medium become frozen because the cryopreservation medium is subjected to a freezing temperature, since there is no step of drying, the cryopreservation medium having anti-cancer drug-loaded platelets when thawed is in a liquid state. In some embodiments, a composition comprising anti-cancer drug-loaded platelets herein upon storing for at least 1 month, 2, 3, 4, 5, 6, 7, 8, 9, 10, 11, 12 months, 1 year, 2, 4, 5, 8, or 10 years at a temperature in the range of −10° C. to −40° C. is capable of exhibiting a platelet count of at least 1.0×10¹¹, 1.2×10¹¹, 1.4×10¹¹, 1.6×10¹¹, or 1.7×10¹¹/20-35 ml of the composition, and retains at least 30%, 35%, 40%, 45%, 50%, 60%, or 70% of the anti-cancer drug upon thawing. For example, a composition herein upon storing for at least 1 month, 2, 3, 4, 5, 6, 7, 8, 9, 10, 11, 12 months, 1 year, 2, 4, 5, 8, or 10 years at a temperature in the range of −10° C. to −40° C. is capable of exhibiting a platelet count of at least 1.0×10¹¹ in a cryo-vessel, cryo-vial, or a cryo-bag having a volume of 35, 30, 25, or 20 ml, or a volume of around 20-35 ml. Platelet counts can be performed with an automated hematology analyzer, or manually with a hemocytometer. For example, platelet counts of a sample, such as a thawed platelet sample can be determined by using a hematology analyzer, for example, a Beckman Coulter AcT Diff 2 Hematology Particle Analyzer or a Beckman Coulter D×H Hematology Analyzer (Beckman Coulter, beckmancoulter.com). Hematology analyzers are known to be based on the Coulter Principle, which is an electronic method for counting and sizing particles. Although the Coulter Principle can be used to calculate and size many types of particles, the specific application of this principle in hematology is to count and size white blood cells (WBC), red blood cells (RBC), and platelets (PLT). As a non-limiting method, the platelet count in a composition herein can be derived from an internal continuous PLT/RBC histogram. Particles between 0 and 70 fL are counted and sized as they pass through the RBC aperture. The raw data is evaluated using a proprietary platelet algorithm, such as D×H (available on the Beckman Coulter D×H Hematology Analyzer) to identify the platelet population. The system also performs feature analysis to identify patterns of interference at the low and high ends of the PLT histogram. The algorithm uses both the PLT raw data and the fitted histograms for this process to determine PLT interference patterns, correcting or flagging results, depending on the severity of the interference. The platelet histogram's evaluation improves accuracy by excluding interferences from debris, micro bubbles, red cell fragments or exceptionally small red blood cells. In some embodiments, platelets or anti-cancer drug-loaded platelets can be counted by considering platelets having a diameter in the range of 0.5-5 μm, 1-4 μm, 1-3 μm, 1-2.5 μm, 1.5-3 μm, or in illustrative embodiments, 0.5-2.5 μm, or 2.5-5.0 μm, typically when measured by flow cytometry or light scattering. In some embodiments, platelets or anti-cancer drug-loaded platelets can be counted by considering platelets having a diameter of at least 0.5 μm, or at least 1 μm, typically when measured by flow cytometry or light scattering. In some embodiment, particles in a composition that are less than 1 μm in diameter are microparticles, typically when measured by flow cytometry or light scattering. In some embodiment, particles in a composition that are less than 0.5 μm in diameter are microparticles, typically when measured by flow cytometry or light scattering. As a non-limiting example of platelet count techniques, flow cytometry can be used for sorting and counting platelets, or platelet derivatives in a composition herein. As is known in the art, different techniques are available for measuring particle sizes of platelets, platelet derivatives, and microparticles, for example platelet derived microparticles. One such technique, in a non-limiting manner, that can be used for measuring particle sizes is flow cytometry. Flow Cytometry is a technique for quantifying characteristics of cells such as cell number, size and complexity, fluorescence, phenotype, and viability. In general, the forward scatter in a flow cytometry is located in line with the laser intercept and is typically considered a measure of the relative cell size. The side scatter is typically located perpendicular to the laser beam intercept and is used to measure the relative complexity of the cell. Commercially available sizing beads can be used to obtain the forward scatter values to calibrate the instrument in order to measure the sizes of the particles. The gates used to measure the size distribution of particles in a composition as disclosed herein are drawn using forward scatter height (FSC—H) signals generated by latex beads of a known diameter. For example, commercially available sizing beads of 0.5 μm, and 2.5 μm can be used to set size gate ranges in a flow cytometry equipment for counting particles that are below 0.5 μm, such as microparticles or platelet derived microparticles, for counting platelets or platelet derivatives that fall in the range of 0.5 μm and 2.5 μm. In some embodiments, a composition comprising frozen anti-cancer drug-loaded platelets or frozen anti-cancer drug-loaded platelet derivatives, or cryopreserved anti-cancer drug-loaded platelets or cryopreserved anti-cancer drug-loaded platelet derivatives in a frozen state, in illustrative embodiments upon storing for at least 1 month, 2, 3, 4, 5, 6, 7, 8, 9, 10, 11, 12 months, 1 year, 2, 4, 5, 8, or 10 years at a temperature in the range of −10° C. to −40° C., upon thawing exhibit a platelet count recovery of at least 65%, 70%, or 75%, and upon thawing retains at least 30%, 35%, 40%, 45%, 50%, 60%, or 70% of the anti-cancer drug. For example, a platelet count recovery can be in a range of 60% to 95%, 65% to 95%, 70% to 95%, or 75% to 95%, 70% to 99%, 72% to 99%, or 75% to 99%. For example, retention of anti-cancer drug, in illustrative embodiments, a chemotherapy drug, for example doxorubicin, can be in the range of 25-99%, 25-95%, 25-90% 25-85%, 25-80%, 25-75%, 25-70%, 25-65%, 25-60%, 30-95%, 30-80%, 35-95%, 35-90%, 35-85%, 35-80%, 35-75%, or 35-70%. A skilled artisan can understand that platelet recovery can be performed by comparing the platelet counts in a composition before freezing and after thawing, to assess the counts after a storage time. In a non-limiting example, percentage platelet recovery can be assessed by using Beckman Coulter AcT Diff 2 Hematology Particle Analyzer or the Beckman Coulter D×H Hematology Analyzer.

In some embodiments, a composition comprising frozen platelets and/or platelet derivatives herein upon storing for at least 1 month, 2, 3, 4, 5, 6, 7, 8, 9, 10, 11, or 12 months at a temperature in the range of −10° C. to −40° C., upon thawing can have a CD61-positive microparticle content of less than 80%, 75%, 70%, 65%, 60%, in illustrative embodiments, less than 50%, 40%, 30%, or 25%. In some embodiments, CD61-positive microparticle content out of all the particles including platelets, platelet derivatives, and microparticles is in the range of 1-30%, 1-25%, 1-20%, 1-15%, 5-30%, 5-25%, or 5-20%. In some embodiments, microparticles, or CD 61-positive microparticles are particles that are less than 0.5 μm in diameter, typically when measured by flow cytometry or light scattering. In some embodiments, microparticles, or CD 61-positive microparticles are particles that are less than 0.25 μm in diameter, typically when measured by flow cytometry or light scattering. In some embodiments, microparticles, or CD 61-positive microparticles are particles that are less than 1 μm in diameter, typically when measured by flow cytometry or light scattering. In some embodiments, at least 70%, 75%, 80% of the particles, typically including platelet, platelet derivatives, and microparticles in the composition are positive for lactadherin. For example, lactadherin positive particles in a composition can be in the range of 70% to 99%, 75% to 99%, or 80% to 99% of the particles in the composition. In some embodiments, lactadherin positive microparticles in a composition can be in the range of 70% to 99%, 75% to 99%, or 80% to 99% of the particles in the composition. Analysing using flow cytometry-based sorting and counting is a non-limiting technique for calculating the percentage positivity of CD-61 positive microparticles, and lactadherin positive particles. Various known techniques can be used to determine the sizes of various populations of particles in a composition as disclosed herein. For example, in some embodiments, flow cytometry forward scattering is used for determining the size of the particles. In other embodiments, light scattering, such as Thrombolux Dynamic light scattering is used for determining the size of the particles.

In some embodiments, a composition comprising frozen platelets, and/or platelet derivatives provided herein, in illustrative embodiments upon thawing, comprises platelets and/or platelet-derived particles, such as platelet derivatives having a particle size (e.g., diameter or max dimension) of at least at least about 0.4 μm, at least about 0.5 μm, at least about 0.6 μm, at least about 0.7 μm, at least about 0.8 μm, at least about 0.9 μm, or at least about 1.0 μm, about 0.5 μm to about 5.0 μm. In some embodiments, the cryopreserved platelet composition has about 50% to about 99% (e.g., about 55% to about 95%, about 60% to about 90%, about 65% to about 85, about 70% to about 80%) of platelets and/or platelet-derived particles in the range of about 0.3 μm to about 5.0 μm in diameter, about 0.5 μm to about 5.0 μm, (e.g., from about 0.4 μm to about 4.0 μm in diameter, from about 0.5 μm to about 2.5 μm in diameter, from about 0.6 μm to about 2.0 μm in diameter, about 1 μm to about 5.0 μm in diameter, about 1 μm to about 4.0 μm in diameter, about 1.5 μm to about 4.5 μm in diameter, or about 1 μm to about 3.0 μm in diameter).

In some embodiments, a composition comprising frozen platelets and/or platelet derivatives, or cryopreserved platelets and/or platelet derivatives herein upon storing for at least 1 month, 2, 3, 4, 5, 6, 7, 8, 9, 10, 11, or 12 months at a temperature in the range of −10° C. to −40° C., upon thawing can exhibit an ability to generate thrombin in an in vitro thrombin generation assay. A skilled artisan can use any known test(s) to assess thrombin generation. For example, thrombin generation can be assessed by a thrombin generation assay, and the assay can be performed by semi-automated methods for example using a calibrated automated thrombogram, or using fully automated systems. Thrombin generation assay is a type of coagulation test and is based on the potential of plasma to generate thrombin over time, following addition of activators like phospholipids, tissue factor, and calcium. The results of the assay can typically be calculated as a thrombogram, or thrombin generation curve using computer software after calculation of thrombogram parameters. A non-limiting example of assay conditions of a thrombin generation assay include incubating platelets in the presence of tissue factor, and phospholipids. In some embodiments, an in vitro assay comprises incubating platelets and/or platelet derivatives in the presence of tissue factor and phospholipids but in the absence of fresh platelets. Thus, in some embodiments, frozen platelets and/or platelet derivatives as disclosed herein can be capable of generating thrombin, for example, when in the presence of a reagent containing tissue factor and phospholipids in vitro. For example, in some cases, frozen platelets and/or platelet derivatives, or cryopreserved platelets and/or platelet derivatives (e.g., at a concentration of at least about 10×10³ particles/μL, 20×10³ particles/μL, 30×10³ particles/μL, or 44×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM), 50 nM, 75 nM, 100 nM, 150 nM, 175 nM, 200 nM, 250 nM, 275 nM, 300 nM, in illustrative embodiments, when in the presence of a reagent containing tissue factor (e.g., at 0.25 pM, 0.5 pM, 1 pM, 2 pM, 5 pM or 10 pM) and optionally phospholipids. For example, in some cases, frozen platelets and/or platelet derivatives, or cryopreserved platelets and/or platelet derivatives (e.g., at a concentration of at least about 10×10³ particles/μL, 20×10³ particles/μL, 30×10³ particles/μL, or 44×10³ particles/μL) as described herein can generate a TPH of about 100 nM to about 350 nM (e.g., about 125 nM to about 350 nM, or about 150 to about 350 nM), in illustrative embodiments when in the presence of a reagent containing tissue factor and (e.g., at 0.25 pM, 0.5 pM, 1 pM, 2 pM, 5 pM or 10 pM) and optionally phospholipids. In some cases, frozen platelets and/or platelet derivatives, or cryopreserved platelets and/or platelet derivatives (e.g., at a concentration of about 4.8×10³ particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives, or cryopreserved platelets and/or platelet derivatives. In some cases, frozen platelets and/or platelet derivatives (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ particles/μL of frozen platelets and/or platelet derivatives. In some embodiments, a composition herein can have an IU of at least 0.4, 0.5, 0.7/10⁶ particles. A skilled artisan can use other known techniques to assess the thrombin generation potential of the platelets, or platelet derivatives as disclosed herein. Accordingly, in some embodiments, a composition herein comprises platelets, or platelet derivatives that retain hemostatic abilities even upon storing at a temperature in the range of −10° C. to −30° C. for at least 12 months.

In some embodiments, a composition comprising frozen platelets and/or platelet derivatives, or cryopreserved platelets and/or platelet derivatives herein when stored at a temperature in the range of −10° C. to −40° C., in illustrative embodiments, upon storing for at least 1 month, 2, 3, 4, 5, 6, 7, 8, 9, 10, 11, or 12 months, upon thawing can be capable of occluding a collagen-coated microchannel, a tissue factor-coated microchannel, or a collagen- and tissue factor-coated microchannel in vitro. For example, such occluding can be determined, for example, by using a total thrombus-formation analysis system (T-TAS®). In some embodiments, a microchannel is collagen-coated microchannel. In some embodiments, a microchannel is tissue factor-coated microchannel, for example, thromboplastin-coated microchannel. In some embodiments, a microchannel is collagen- and tissue factor-coated microchannel. In some cases, frozen or cryopreserved platelets or platelet derivatives as described herein upon thawing, when at a concentration of at least 50×10³ particles/μL, 60×10³ particles/μL, or 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 60) of less than 30, 25, 20, 15, or 14 minutes, or between 5 on the low end of the range, and 15, 20, or 25 on the high end, or between 10 on the low end of the range, and 15, 20, or 25 on the high end, or between 15 on the low end of the range and 20 or 25 on the high end, for example, in platelet-reduced citrated whole blood. In some cases, frozen or cryopreserved platelets or platelet derivatives as described herein upon thawing, when at a concentration of at least 50×10³ particles/μL, 60×10³ particles/μL, or 70×10³ particles/μL (e.g., at least 73×10³, 100×10³, 150×10³, 173×10³, 200×10³, 250×10³, or 255×10³ particles/μL) can result in an area under the curve (AUC) of at least 1300 (e.g., at least 1380, 1400, 1500, 1600, or 1700), for example, in platelet-reduced citrated whole blood. The occlusion time depicts the time it takes the sample to form a thrombus. The lower the time the faster the thrombus formation occurred. The analysis can capture occlusion time (OT) and area under the curve (AUC). OT represents the lag time it takes for the flow pressure to reach a target pressure, such as 60 kPa, 70 kPa, or 80 kPa from the baseline pressure. The AUC is the area under the flow pressure versus time curve which is related to overall thrombus formation. Microchannels or capillaries having different dimensions can be used in a T-TAS system for determining the occlusion times of cryopreserved platelets or cryopreserved platelet derivatives, or frozen platelets or frozen platelet derivatives under different experimental conditions as provided by numerous commercial suppliers (See e.g., Zacros, Tokyo, JP). For example, a T-TAS PL chip, AR chip, or HD chip can be used for an occlusion (e.g., T-TAS) assay, as are commercially available. Typically, an AR chip for the purposes of T-TAS assay is coated with either collagen, or a tissue-factor, such as thromboplastin, or both. Typically an HD chip for the purposes of T-TAS assay is coated with either collagen, or a tissue-factor, such as thromboplastin, or both. For example, the PL chip can have capillary dimensions of 40 μm×40 μm; or an AR chip can have capillary dimensions of 0.3 mm×80 μm; or an HD chip can have capillary dimensions of 0.3 mm×50 μm. Therefore, it is envisioned that a T-TAS assay can be performed to test the ability to occlude a collagen-coated microchannel, utilizing a microchannel or capillary with dimensions in the range of 0.02-0.5, 0.1-0.5, 0.2-0.4, 0.1-0.3, or 0.2-0.3 mm×25-200, 25-100, 50-100, 40-90, 40-80, or 50-80 μm.

In some embodiments, a composition comprising anti-cancer drug-loaded frozen platelets and/or anti-cancer drug-loaded platelet derivatives herein, when stored at a temperature in the range of −10° C. to −40° C., in illustrative embodiments, upon storing for at least 1 month, 2, 3, 4, 5, 6, 7, 8, 9, 10, 11, or 12 months, upon thawing can exhibit a single peak in a membrane integrity assay typically based upon retention of fluorophore in platelets and/or platelet derivatives. A skilled artisan can contemplate different techniques to study the retention of a fluorophore in particles, such as platelets, and/or platelet derivatives. One such technique is Calcein acetoxymethyl (AM) membrane integrity assay. Calcein AM is a substance that is able to cross the cell membrane and reach the cytosol where Calcein AM gets hydrolyzed by the enzyme esterase to produce fluorescence. Platelets and/or platelet derivatives that are intact are able to retain this fluorescence while non-intact platelets do not. Therefore, based on the fluorescence that is emitted particles can be assessed for their membrane integrity. Accordingly, based on Calcein AM assay, in some embodiments, platelets and/or platelet derivatives in a composition provided herein do not have intact cell membranes, i.e. have compromised membranes.

Another non-limiting technique for assessing membrane integrity is by detecting lactate dehydrogenase enzyme (LDH) that is released by the cells having a compromised membrane. LDH is a stable cytoplasmic enzyme that is found in all cells. LDH is rapidly released into the cell culture supernatant when the cell membrane is damaged. According to one of the protocols, LDH activity can be easily quantified by using the nicotinamide adenine dinucleotide (NAD)+hydrogen (NADH) produced during the conversion of lactate to pyruvate to reduce a second compound in a coupled reaction into a product with properties that are easily quantitated. This protocol measures the reduction of a yellow tetrazolium salt, Iodonitrotetrazolium (INT), by NADH into a red, water-soluble formazan-class dye by absorbance at 492 nm. The amount of formazan is directly proportional to the amount of LDH in the supernatant, which is, in turn, directly proportional to the number of cells that have compromised membrane. Accordingly, in some embodiments, frozen platelets or platelet derivatives, or cryopreserved platelets or platelet derivatives in a composition herein have compromised membrane as per LDH assay for assessing membrane integrity.

In some embodiments, a composition comprising frozen anti-cancer drug-loaded platelets and/or anti-cancer drug-loaded platelet derivatives, or cryopreserved anti-cancer drug-loaded platelets and/or anti-cancer drug-loaded platelet derivatives herein when stored at a temperature in the range of −10° C. to −40° C., in illustrative embodiments, upon storing for at least 1 month, 2, 3, 4, 5, 6, 7, 8, 9, 10, 11, or 12 months, upon thawing is capable of showing aggregation under aggregation conditions comprising an agonist, not limited to arachidonic acid, collagen, and TRAP-6. In some embodiments, aggregation conditions comprise an agonist but no fresh or apheresis platelets. In some embodiments, aggregation conditions comprise an agonist but no fresh or apheresis platelets, and no divalent cation. Non-limiting examples of aggregation agonists include, collagen, epinephrine, ristocetin, arachidonic acid, adenosine di-phosphate, and thrombin receptor associated protein (TRAP). In some embodiments, frozen platelets and/or platelet derivatives, or cryopreserved platelets and/or platelet derivatives herein upon thawing exhibit aggregation in the presence of arachidonic acid, in the range of 20-60%, 20-50%, or 30-50%, or aggregation of at least 20%, 30%, 40%, or 50%. In some embodiments, frozen platelets and/or platelet derivatives, or cryopreserved platelets and/or platelet derivatives herein upon thawing exhibit aggregation in the presence of collagen, in the range of 2-50%, 2-40%, or 2-30%. In some embodiments, frozen platelets and/or platelet derivatives, or cryopreserved platelets and/or platelet derivatives herein upon thawing exhibit aggregation in the presence of TRAP-6, in the range of 2-50%, 2-40%, or 2-30%.

### Stability of the Cryopreserved Anti-Cancer Drug-Loaded Platelets

In some embodiments of aspects that include a process for preparing a cryopreserved anti-cancer drug-loaded platelet composition including a transition in freezing temperatures from an initial freezing temperature to a storage freezing temperature, a process for preparing a cryopreserved anti-cancer drug-loaded platelet composition including a single temperature, and a composition comprising cryopreserved anti-cancer drug-loaded platelets, the anti-cancer drug-loaded cryopreserved platelets herein can be stable when stored frozen at −80° C., −70° C., −60° C., −50° C., −40° C., −30° C., −20° C., or higher. In some embodiments, cryopreserved anti-cancer drug-loaded platelets as disclosed herein, or cryopreserved anti-cancer drug-loaded platelets formed by a process as disclosed herein, in illustrative embodiments, cryopreserved anti-cancer drug-loaded platelets formed by a process that comprises freezing anti-cancer drug-loaded platelets in a cryopreservation medium as disclosed herein, at a temperature of less than or equal to −50° C., −55° C., −60° C., in illustrative embodiments, less than or equal to −65° C., −70° C., −75° C., or −80° C., to form an initial frozen anti-cancer drug-loaded platelet composition, and a second step comprising storing the initial frozen anti-cancer drug-loaded platelet composition at a temperature of more than or equal to −40° C., −35° C., −30° C., −25° C., in illustrative embodiments, more than or equal to −20° C., −15° C., or −10° C., but less than 0° C., are stable when stored at a temperature of more than or equal to −40° C., −35° C., −30° C., −25° C., −15° C., or −10° C., but less than 0° C. Stability of the cryopreserved anti-cancer drug-loaded platelets present in a cryo-vessel as provided in a collection of cryo-vessels herein, can be assessed by a non-limiting list of parameters including visual inspection of cracks, tears, breaks of the cryo-vessel, such as a cryo-bag, visual inspection of aggregate free swirling of the cryopreserved anti-cancer drug-loaded platelets in the cryo-vessel, such as a cryo-bag, platelet counts per cryo-vessel, and pH of the cryopreserved anti-cancer drug-loaded platelets in the cryo-vessel. For example, in some non-limiting embodiments, cryopreserved anti-cancer drug-loaded platelets herein are stable when stored at a specific temperature, for example, at about −20° C., or higher when the cryopreserved anti-cancer drug-loaded platelets swirl without the presence of any aggregation on a visual inspection. For example, when stored at −20° C.+/−10° C., −20° C.+/−8° C., −20° C.+/−5° C., or −20° C.+/−2° C. the cryopreserved anti-cancer drug-loaded platelets swirl without the presence of any aggregation on a visual inspection. For example, in some non-limiting embodiments, cryopreserved anti-cancer drug-loaded platelets herein are stable when stored at a specific temperature, for example, at about −20° C., or higher, for example, stored at −20° C.+/−5° C. the pH of the cryopreserved anti-cancer drug-loaded platelets, typically upon thawing is equal to or more than 6.0, typically, equal to or more than 6.2. For example, the cryopreserved anti-cancer drug-loaded platelets herein upon storing at about −20° C., for example, at −20° C.+/−5° C. for a period in the range of 1 month-36 months, 1 month-30 months, 1 month-24 months, 1 month-18 months, or 1 month-12 months, typically upon thawing exhibit a pH higher than 7.0. In some embodiments, the cryopreserved anti-cancer drug-loaded platelets herein upon storing at about −20° C., for example, at −20° C.+/−5° C. for a period in the range of 1-12 months, typically upon thawing exhibit a pH higher than 6.2, 6.4, 6.6, 6.8, 7.0, or 7.2. For example, the cryopreserved anti-cancer drug-loaded platelets herein upon storing at −20° C.+/−5° C. for a period in the range of 1-12 months, typically upon thawing exhibit a pH in the range of 6.2 to 7.8, 6.4 to 7.8, 6.6 to 7.8, or 7-7.8.

For example, in some non-limiting embodiments, cryopreserved anti-cancer drug-loaded platelets herein are stable when stored at a specific temperature, for example, at about −20° C., or higher, for example, stored at −20° C.+/−5° C. the total number of platelets, typically upon thawing in a cryo-vessel is equal to or more than 1.5×10¹¹, 1.6×10¹¹, or 1.7×10¹¹. In illustrative embodiments, cryopreserved anti-cancer drug-loaded platelets herein, typically when stored at −20° C.+/−5° C. for at least 1 month, 2, 3, 4, 6, 8, 10, 12 months, 2, 3, 4, 5, 6, 7, 8, 9, or 10 years, upon thawing in some embodiments have a total number of platelets in a cryo-vessel equal to or more than 1.5×10¹¹, 1.6×10¹¹, or 1.7×10¹¹, and upon thawing retains at least 30%, 35%, 40%, 45%, 50%, 60%, or 70% of the anti-cancer drug. In some embodiments, cryopreserved anti-cancer drug-loaded platelets herein, typically upon thawing exhibit a particle size, for example, diameter in the range of 0.5 μm to 2.5 μm, 1 μm to 5 μm, 1 μm to 4 μm, or 0.5 μm to 5.0 μm such that at least 50% of the platelets after thawing have a diameter in the range of 0.5 μm to 2.5 μm, 1 μm to 5 μm, 1 μm to 4 μm, or 0.5 μm to 5.0 μm. In some embodiments, cryopreserved anti-cancer drug-loaded platelets herein, in illustrative embodiments, upon storing at a temperature in the range of −40° C. to −5° C., −30° C. to −5° C., or −20° C. to −5° C., for at least 1 month, 2, 3, 4, 6, 8, 10, 12 months, 2, 3, 4, 5, 6, 7, 8, 9, or 10 years typically upon thawing exhibit a particle size, for example, diameter in the range of 0.5 μm to 2.5 μm, 1 μm to 5 μm, 1 μm to 4 μm, or 0.5 μm to 5.0 μm and upon thawing retains at least 30%, 35%, 40%, 45%, 50%, 60%, or 70% of the anti-cancer drug, and in some embodiments, have a total number of anti-cancer drug-loaded platelets in a cryo-vessel equal to or more than 1.5×10¹¹, 1.6×10¹¹, or 1.7×10¹¹. In some embodiments, at least 50%, 60%, 70%, or 75% of the cryopreserved anti-cancer drug-loaded platelets upon thawing have a diameter in the range of 0.5 μm to 2.5 μm, 1 μm to 5 μm, 1 μm to 4 μm, or 0.5 μm to 5.0 μm. In illustrative embodiments, cryopreserved anti-cancer drug-loaded platelets herein upon thawing have a total number of platelets in a cryo-vessel equal to or more than 1.5×10¹¹, 1.6×10¹¹, or 1.7×10¹¹, and typically, the cryopreserved anti-cancer drug-loaded platelets upon thawing retain hemostatic properties, for example, generating thrombin in an in vitro condition, ability to reduce bleeding in a subject, or ability to increase platelet numbers in a subject in need thereof, and also retains at least 30%, 35%, 40%, 45%, 50%, 60%, or 70% of the anti-cancer drug. In illustrative embodiments, the ability to reduce bleeding in a subject is based on administration of, for example 0.5 to 3 units of frozen platelets or frozen anti-cancer drug-loaded platelets, frozen platelet derivatives or frozen anti-cancer drug-loaded platelet derivatives, cryopreserved platelets or cryopreserved anti-cancer drug-loaded platelets, or cryopreserved platelet derivatives or cryopreserved anti-cancer drug-loaded platelet derivatives. In illustrative embodiments, where 1 unit corresponds to 2.5×10¹¹+/−4.2×10¹¹ frozen or cryopreserved platelets and/or platelet derivatives, or frozen or cryopreserved anti-cancer drug-loaded platelets and/or platelet derivatives. In some embodiments, methods herein include administering liquid compositions of thawed compositions of frozen platelets or frozen anti-cancer drug-loaded platelets, frozen platelet derivatives or frozen anti-cancer drug-loaded platelet derivatives, cryopreserved platelets or cryopreserved anti-cancer drug-loaded platelets, or cryopreserved platelet derivatives or cryopreserved anti-cancer drug-loaded platelet derivatives provided herein and/or prepared according to any method provided herein, to a subject to restore hemostasis, reduce bleeding, or stop bleeding in the subject, or to reduce the size of a tumor in a subject, in illustrative embodiments, thereby treating cancer in the subject.

Stability can also be determined by assessing certain parameters after thawing the cryopreserved platelets or cryopreserved anti-cancer drug-loaded platelets that are stored at a temperature of more than or equal to −40° C., −35° C., −30° C., −25° C., −15° C., or −10° C., but less than 0° C., in illustrative embodiments, at a temperature in the range of −40° C. to −10° C. In some embodiments, thawing of cryopreserved platelets or cryopreserved anti-cancer drug-loaded platelets as disclosed herein, or cryopreserved platelets or cryopreserved anti-cancer drug-loaded platelets obtained by a process as disclosed herein, can be done by subjecting the cryopreserved platelets or cryopreserved anti-cancer drug-loaded platelets to a temperature above the freezing temperature. For example, subjecting the cryopreserved platelets or cryopreserved anti-cancer drug-loaded platelets to a temperature above 0° C., for example, at least 5° C., 10° C., 15° C., 20° C., 25° C., 30° C., 35° C. In Illustrative embodiments, thawing comprises subjecting the cryopreserved platelets or cryopreserved anti-cancer drug-loaded platelets to a temperature in the range of 20° C. to 40° C., 22° C. to 40° C., 25° C. to 40° C., 30° C. to 40° C., or 32° C. to 40° C. In some embodiments, thawing comprises subjecting the cryopreserved platelets or cryopreserved anti-cancer drug-loaded platelets to a temperature of 37° C.+/−5° C., 37° C.+/−4° C., 37° C.+/−3° C., 37° C.+/−2° C., 37° C.+/−1° C., or 37° C.+/−0.5° C. Thawing herein typically comprises subjecting a cryo-vessel or a cryo-vial having cryopreserved platelets or cryopreserved anti-cancer drug-loaded platelets as disclosed herein to a water-bath set at a temperature of 37° C.+/−2° C. for a time-period until the contents in the cryo-vessel are completely thawed. A skilled artisan can contemplate that the time required for the contents to thaw completely can vary according to the volume of cryopreserved platelets or cryopreserved anti-cancer drug-loaded platelets, dimensions of the cryo-vessel and the temperature at which the cryo-vessels were stored before the thawing. Accordingly, thawing can be done by subjecting cryo-vessels to a water-bath set at a temperature of 37° C.+/−2° C. for at least 1 minute, 2, 3, 4, 5, 6, 7, 8, 9, or 10 minutes, for example in a range of 2-10, 2-9, 2-8, 2-7, or 2-6 minutes.

### Preparation of Cryopreserved Anti-Cancer Drug-Loaded Platelets Using a Single Freezing Temperature

A skilled artisan will understand that a process including a single freezing temperature for freezing anti-cancer drug-loaded platelets to form cryopreserved or frozen anti-cancer drug-loaded platelets and a single-temperature cryopreserved anti-cancer drug-loaded product can in fact, be subjected to a variation in temperatures, but such variation does not include a transition from an initial freezing temperature at or below −50° C. to a target storage temperature at −10° C. to −30° C.

In some aspects, provided herein is a method for preparing cryopreserved anti-cancer drug-loaded platelets that include a single freezing temperature. For example, a method herein can comprise freezing a population of anti-cancer drug-loaded platelets obtained by methods as disclosed herein, at a temperature of between −15° C. to −85° C., in illustrative embodiments between −50° C. to −85° C., to form cryopreserved anti-cancer drug-loaded platelets, and storing cryopreserved anti-cancer drug-loaded platelets at a temperature of between −15° C. to −85° C., in illustrative embodiments, between −50° C. to −85° C. for at least 1 day, 2, 4, 6, 7, 8, 10, 12, 14, 16, 18, 20, 25 days, 1 month, or 2 months. In illustrative embodiments, a method herein can comprise freezing a population of anti-cancer drug-loaded platelets obtained by methods as disclosed herein, at a temperature of between −50° C. to −85° C., to form cryopreserved anti-cancer drug-loaded platelets, and storing cryopreserved anti-cancer drug-loaded platelets at a temperature of between −50° C. to −85° C. for at least 1 day, 2, 4, 6, 7, 8, 10, 12, 14, 16, 18, 20, 25 days, 1 month, 2 months, 3, 4, 6, 8, 10, 12 months, 2, 4, or 5 years. In some embodiments, cryopreserved anti-cancer drug-loaded platelets herein after storing for at least 18 days at a temperature in a range of −50° C. to −85° C. can retain at least 50%, 60%, or 70% of the anti-cancer drug. In some embodiments, wherein the anti-cancer drug is doxorubicin, cryopreserved doxorubicin-loaded platelets after storing for at least 18 days at a temperature in a range of −50° C. to −85° C. can retain at least 50%, 60%, or 70% of doxorubicin. In some embodiments, cryopreserved anti-cancer drug-loaded platelets are stored at a temperature in the range of −75° C. to −85° C. for a time period in a range of between 2, 3, 4, 5, 6, 7, 10, or 14 days on the low end of the range, and 28 days on the high end, between 2, 3, 4, 5, 6, 7, 10, or 14 days on the low end of the range, and 20 days on the high end, between 2 days on the low end of the range, and 7, 8, 10, 14, 18, 28, 30, 45, or 60 days on the high end, between 7 days on the low end of the range, and 8, 10, 14, 18, 28, 30, 45, or 60 days on the high end, or between 10 days on the low end of the range, and 14, 18, 28, 30, 45, or 60 days on the high end. In some embodiments, cryopreserved anti-cancer drug-loaded platelets herein after the storing are administered to a subject having cancer.

### Platelet Derivatives, and Anti-Cancer Drug-Loaded Platelet Derivatives

In some aspects, provided herein is a method for preparing anti-cancer drug-loaded platelet derivatives comprising: obtaining anti-cancer drug-loaded platelets by a process as disclosed herein, lyophilizing the anti-cancer drug-loaded platelets to form anti-cancer drug-loaded platelet derivatives, and storing the anti-cancer drug-loaded platelet derivatives for at least 2, 3, 4, 5, 7, 8, 10, 12, 14, 18, or 20 days at a temperature between 4° C. to 30° C., 8° C. to 30° C., 10° C. to 30° C., 15° C. to 30° C., 20° C. to 30° C., 22° C. to 30° C., or 22° C. to 28° C. Typically, a method herein including lyophilizing does not include cryopreserving. In some embodiments, the anti-cancer drug-loaded platelet derivatives herein can be stored at a room temperature until used for administering to a subject. In some embodiments, temperatures at which anti-cancer drug-loaded platelet derivatives can be stored can be more than 0° C. to less than 45° C., for example, between 0° C. and 10° C., 2° C. and 10° C., 3° C. and 10° C., 0° C. and 7° C., or 0° C. and 5° C. For example, between 5° C. to less than 40° C., 10° C. to 40° C., or 20° C. to 40° C. The anti-cancer drug-loaded platelet derivatives obtained as per a method herein can further be used for treating cancer in a subject by administering a therapeutically effective amount of the anti-cancer drug-loaded platelet derivatives. In some embodiments, the anti-cancer drug-loaded platelet derivatives herein can retain at least 25%, 30%, 35%, 40%, 45%, 50%, 55%, or 60% of the anti-cancer drug. For example, the anti-cancer drug-loaded platelet derivatives herein can retain between 25% to 99%, 25% to 95%, 25% to 90%, 25% to 85%, 25% to 80%, 25% to 75%, 25% to 70%, 30% to 99%, 35% to 99%, 40% to 99%, or 45% to 99%. In some embodiments, the anti-cancer drug-loaded platelet derivatives herein can be stored for at least 12, 14, 18, 20, 25, 28, 1 month, 2, 4, 6, 8, 10, 12 months, 2, 4, 6, 8, or 10 years, and in illustrative embodiments can retain at least 40%, or 50% of the anti-cancer drug.

In some embodiments, the dried platelets, such as freeze-dried platelet derivatives, for example, anti-cancer drug-loaded platelet derivatives have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets, have less than about 10%, such as less than about 8%, such as less than about 6%, such as less than about 4%, such as less than about 2%, such as less than about 0.5% crosslinking of platelet membranes via proteins and/or lipids present on the membranes.

Platelet derivatives, or anti-cancer drug-loaded platelet derivatives herein have been observed to have numerous surprising properties, as disclosed in further detail herein. It will be understood, as illustrated in the Examples of U.S. Pat. No. 11,529,587 and PCT app no. PCT/US2022/079280, that although platelet derivatives in some aspects and embodiments are in a solid, such as a powder form, the properties of such platelet derivatives can be identified, confirmed, and/or measured when a composition comprising such platelet derivatives is in liquid form. In some embodiments, methods for preparing platelet derivatives or anti-cancer drug-loaded platelet derivatives herein can include tangential flow filtration method as disclosed in U.S. Pat. No. 11,529,587, incorporated by reference herein, in its entirety. In some embodiments, methods herein can include obtaining platelets by tangential flow filtration method as disclosed in U.S. Pat. No. 11,529,587.

A skilled artisan would be well-versed with different techniques that are available for measuring particle sizes of platelets, platelet derivatives, anti-cancer drug-loaded platelet derivatives or FDPDs, and microparticles. One such technique, in a non-limiting manner, that can be used for measuring particle sizes is flow cytometry. Flow Cytometry is a technique for quantifying characteristics of cells such as cell number, size and complexity, fluorescence, phenotype, and viability. In general, the forward scatter in a flow cytometry is located in line with the laser intercept and is typically considered a measure of the relative cell size. The side scatter is typically located perpendicular to the laser beam intercept and is used to measure the relative complexity of the cell. Commercially available sizing beads can be used to obtain the forward scatter values to calibrate the instrument in order to measure the sizes of the particles.

In illustrative embodiments, illustrative or target platelet derivatives are CD41 positive and/or CD42 positive. Platelets derivatives (e.g., thrombosomes) herein, in some embodiments, are dry platelet derivatives, or dry platelet derived particles. A skilled artisan will understand that most properties of such dry platelet derivatives are analyzed after the platelet derivatives are rehydrated. Dry platelet derivatives are typically present in a dried substance that includes other components (e.g., saccharides such as, for example, trehalose and/or polysucrose) present along with the platelet derivatives when they were dried. In illustrative platelet derivative compositions herein, less than 5% of the particles are microparticles having a diameter of less than 0.5 μm. In illustrative platelet derivative compositions herein, at least 90% of the particles therein are at least 0.5 μm in diameter. Furthermore, in illustrative embodiments, between 75% and 95% of the platelet derivatives or particles therein are CD41 positive, between 75% and 95% of the platelet derivatives or particles therein are CD42 positive, and less than 5% of the CD 41-positive platelet derivatives or particles therein are microparticles having a diameter of less than 0.5 μm. It will be understood that in such percent calculations, particles are only intended to cover those that can be detected for example by the instrument (e.g., flow cytometer) used to detect CD41 or CD42 or any surface marker.

In some examples of such illustrative embodiments, the platelet derivatives have a potency of at least 1.5 thrombin generation potency units (TGPU) per 10⁶ platelet derivatives. In non-limiting illustrative embodiments, FDPD compositions, or illustrative anti-cancer drug-loaded platelet derivatives herein, such as those prepared according to Example 1 herein, are compositions that include illustrative or target platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and typically such compositions have the ability to generate thrombin in an in vitro thrombin generation assay and/or have the ability to occlude a collagen-coated microchannel in vitro. In illustrative embodiments, the platelet derivatives in such compositions have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the illustrative or target platelet derivatives are between 0.5 and 2.5 μm in diameter by flow cytometry or between 0.5 and 25.0 μm in diameter by dynamic light scattering.

Liquid and dried compositions provided herein, in illustrative embodiments those prepared using freeze drying, and more specifically in some embodiments, prepared using methods provided herein, include particles that can be categorized broadly into populations based on at least one physical property, for example, but not limiting to, the size of the particles obtained. In some embodiments, the particles can be categorized into two populations based on size, typically in embodiments where exosomes are not present in detectable quantities, are not resolvable by the instrument analyzing particle size, and/or are not considered particles: For example, a first population comprising larger particles similar, or much more similar in size to in-dated stored platelets, which can be referred to herein as platelet derivatives, the anti-cancer drug-loaded platelet derivatives herein, FDPDs, platelet-sized particles, a population of platelet derivatives with a size distribution centered around ˜1,000 nm radius, or ˜1,000 nm radius particles, and a second population comprising relatively smaller particles, which can be referred to herein as microparticles, a population of microparticles with a size distribution centered around ˜50 nm radius, or ˜50 nm radius particles (See e.g., FIG. 24A, FIG. 27B, or FIG. 28B, of U.S. Pat. No. 11,529,587 and of PCT app no. PCT/US2022/079280, where the main population of human in-dated stored platelets is centered at around 1,100 or 1,500 nm radius respectively, with a smaller (i.e. microparticle) peak at around 100 nm radius; and the particles in the FDPD composition (i.e. thrombosomes), which have a ˜platelet-sized major peak with a radius of approximately 1,100 nm (FIG. 24A) or 1,000 nm (FIG. 27B and FIG. 28B) (i.e. platelet derivatives) and a microparticle radius peak at approximately 50-75 nm).

A skilled artisan would further understand that the sizes determined for such populations of particles may not always be accurate enough to provide an exact cut-off value/range between these two particle size peaks. However, the difference in the sizes of the two populations can be resolved reproducibly using known methods, for example, using flow cytometry, or by using a particle/cell counter. And approximate size values or size range values can be obtained using such techniques optionally with sizing standards. In some embodiments, a composition comprising platelet derivatives or FDPDs, or anti-cancer drug-loaded platelet derivatives as described herein or prepared according to methods described herein can have a population comprising platelet derivatives or FDPDs, or anti-cancer drug-loaded platelet derivatives that includes between 75 and 99.9%, or in certain illustrative embodiments between 95.1% to 99.9% of total particles in the composition having a radius of at least 200 nm, and the rest of the measurable platelet derivatives, for example above 1 nm radius, are microparticles. In some embodiments, platelet derivatives or FDPDs, or anti-cancer drug-loaded platelet derivatives in such a composition can have a diameter of at least 0.4 μm (i.e., radius of at least 200 nm), and the microparticles in such a composition can have a diameter less than 0.4 μm (i.e., radius less than 200 nm). In other embodiments, platelet derivatives or FDPDs, or anti-cancer drug-loaded platelet derivatives in such a composition can have a diameter of at least 0.5 μm (i.e., radius of at least 250 nm), and the microparticles in such a composition can have a diameter less than 0.5 μm (i.e., radius of less than 250 nm). In some embodiments, the platelet derivatives, the anti-cancer drug-loaded platelet derivatives herein, or FDPDs can have a diameter of at least 0.4 μm, for example in the range of 0.5 μm to 22 μm (i.e., radius in the range of 200 nm or 250 nm to 11,000 nm), and the microparticles can have a diameter less than 0.5 μm (i.e., less than 250 nm radius), for example in the range of 0.04 μm to 0.350 μm (i.e., radius in the range of 20 nm to 175 nm). In some embodiments, the platelet derivatives, the anti-cancer drug-loaded platelet derivatives herein, or FDPDs can have a diameter in the range of 1 μm to 18 μm (i.e., radius in the range of 500 nm to 9,000 nm), and the microparticles can have a diameter in the range of 0.06 μm to 0.2 μm (i.e., radius in the range of 30 nm to 100 nm). In some embodiments, the composition comprises platelet derivatives or FDPDs, or the anti-cancer drug-loaded platelet derivatives herein, and microparticles as the only or essentially the only particles present in the composition, optionally or typically other than exosomes, in embodiments where exosomes are not present in detectable quantities, are not resolvable by the instrument analyzing particle size, and/or are not considered particles. Of course, a composition as described herein may comprise any specific percentage number, or fraction thereof, of platelet derivatives, the anti-cancer drug-loaded platelet derivatives herein, FDPDs or microparticles within the ranges discussed herein.

In some embodiments, platelets, are pooled from a plurality of donors before being used to make platelet derivatives or cryopreserved platelets herein. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

Compositions comprising platelets or platelet derivatives (e.g., thrombosomes), or anti-cancer drug-loaded platelet derivatives such as those prepared according to methods described herein, can have a microparticle content that contributes to less than about 5.0% (e.g., less than about 4.5%, 4.0%, 3.5%, 3.0%, 2.5%, 2.0%, 1.5%, 1.0%, 0.5%, or 0.1%) of the total scattering intensity of all particles from about 1 nm to about 60,000 nm in radius in the composition. In some embodiments, the platelet derivative composition comprises a population of anti-cancer drug-loaded platelet derivatives comprising CD41-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of anti-cancer drug-loaded platelet derivatives comprising CD42-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of anti-cancer drug-loaded platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. As used herein, a content of microparticles “by scattering intensity” refers to the microparticle content based on the scattering intensity of all particles from about 1 nm to about 60,000 nm in radius in the composition. The microparticle content can be measured by any appropriate method, for example, by dynamic light scattering (DLS). In some cases, the viscosity of a sample used for DLS can be at about 1.060 cP (or adjusted to be so), as this is the approximate viscosity of plasma. In some embodiments, the platelet derivative composition as per any aspects, or embodiments comprises a population of anti-cancer drug-loaded platelet derivatives, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, the platelet derivatives, or anti-cancer drug-loaded platelet derivatives have a diameter in the range of 0.5-2.5 μm, and the microparticles have a diameter less than 0.5 μm.

Platelets or platelet derivatives (e.g., thrombosomes), or anti-cancer drug-loaded platelet derivatives as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×10⁵ thrombosomes/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein IIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, platelets or platelet derivatives (e.g., thrombosomes), or anti-cancer drug-loaded platelet derivatives such as those prepared by methods described herein, can have an average CD41 percent positivity of at least 55% (e.g., at least 60%, at least 65%, at least 67%, at least 70%, at least 75%, at least 80%, at least 85%, at least 90%, or at least 95%). In some embodiments, platelets or platelet derivatives, or anti-cancer drug-loaded platelet derivatives herein can have an average CD41 percent positivity in the range of 70%-99%, 70%-95%, 70%-90%, 70%-86%, or 75%-86%. In some embodiments, at least 90%, 91%, 92%, 93%, 94%, 95%, 96%, 97%, 98%, 99% anti-cancer drug-loaded platelet derivatives that are positive for CD 41 have a size in the range of 0.5-2.5 μm. In some embodiments, at least 90%, 91%, 92%, 93%, 94%, 95%, 96%, 97%, 98%, or 99% anti-cancer drug-loaded platelet derivatives that are positive for CD 41 have a size in the range of 0.4-2.8 μm. In some embodiments, at least 90%, 91%, 92%, 93%, 94%, 95%, 96%, 97%, 98%, or 99% anti-cancer drug-loaded platelet derivatives that are positive for CD 41 have a size in the range of 0.3-3 μm.

As another example, platelets or platelet derivatives (e.g., thrombosomes), or anti-cancer drug-loaded platelet derivatives such as those described herein, can have an average CD42 percent positivity of at least 65% (e.g., at least 67%, at least 70%, at least 75%, at least 80%, at least 85%, at least 90%, or at least 95%). In some embodiments, platelets or platelet derivatives, or anti-cancer drug-loaded platelet derivatives can have an average CD42 percent positivity of at least 76%, 77%, 78%, or 79%. In some embodiments, platelets or platelet derivatives, or anti-cancer drug-loaded platelet derivatives can have an average CD42 percent positivity in the range of 76-95%, 76-94%, 77-93%, or 78-90%. In some embodiments, at least 90%, 91%, 92%, 93%, 94%, 95%, 96%, 97%, 98%, or 99% anti-cancer drug-loaded platelet derivatives that are positive for CD 42 have a size in the range of 0.5-2.5 μm. In some embodiments, at least 90%, 91%, 92%, 93%, 94%, 95%, 96%, 97%, 98%, or 99% anti-cancer drug-loaded platelet derivatives that are positive for CD 42 have a size in the range of 0.4-2.8 μm. In some embodiments, at least 90%, 91%, 92%, 93%, 94%, 95%, 96%, 97%, 98%, or 99% anti-cancer drug-loaded platelet derivatives that are positive for CD 42 have a size in the range of 0.3-3 μm.

As another example, platelets or platelet derivatives (e.g., thrombosomes), or anti-cancer drug-loaded platelet derivatives such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% anti-cancer drug-loaded platelet derivatives that are positive for CD 62 have a size in the range of 0.5-2.5 μm. In some embodiments, at least 70%, 75%, 80%, 85%, 90%, 91%, 92%, 93%, 94%, 95%, 96%, 97%, 98%, or 99% anti-cancer drug-loaded platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% anti-cancer drug-loaded platelet derivatives that are positive for CD 62 have a size in the range of 0.3-3 μm.

As yet another example, platelets or platelet derivatives (e.g., thrombosomes), or anti-cancer drug-loaded platelet derivatives such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm. In some embodiments, at least 90%, 91%, 92%, 93%, 94%, 95%, 96%, 97%, 98%, or 99% platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm. In some embodiments, the presence of phosphatidyl serine in/on the platelet derivatives herein is higher than the presence of phosphatidyl serine in/on the platelets, such as, fresh platelets or apheresis platelets. For example, platelet derivatives herein exhibit at least 5 fold, 10 fold, 20 fold, 25 fold, 30 fold, 40 fold, or 50 fold higher presence of phosphatidyl serine as compared to the platelets.

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP). In some embodiments, the platelet activation marker is selected from the group consisting of Annexin V, and CD 62. In some embodiments, the platelet derivatives as described herein show an inability to increase expression of Annexin V in the presence of TRAP. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

In some embodiments, rehydrating the drug-loaded platelets comprises adding to the platelets an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension. In some embodiments, the rehydrated platelets have coagulation factor levels showing all individual factors (e.g., Factors VII, VIII and IX) associated with blood clotting at 40 international units (IU) or greater.

### Methods for Administering an Anti-Cancer Drug to a Subject

In an aspect, provided herein is a method for administering an anti-cancer drug to a subject, comprising administering a therapeutically effective dose of anti-cancer drug-loaded platelet derivatives, or cryopreserved anti-cancer drug-loaded platelets as disclosed herein including transition temperature-cryopreserved product, and single temperature-cryopreserved product. In some embodiments, the subject has cancer, and in some embodiments, the subject is other than a contraindicated person, wherein the contraindicated person has a stent, has a heart condition, had a diagnosed blood clot, and/or has an effective amount of an anti-platelet agent in their bloodstream. In some embodiments, the subject has cancer, and also has one or more of the following indications: has a stent, has a heart condition, had a diagnosed blood clot, and/or has an effective amount of an anti-platelet agent in their bloodstream. In some embodiments, administering an effective dose of cryopreserved anti-cancer drug-loaded platelets, or anti-cancer drug-loaded platelet derivatives herein can be used for treating cancer in a subject, for reducing tumor load in a subject, or for killing cancer or tumor cells in a subject.

It can be understood that there can be a time-delay in preparation of cryopreserved anti-cancer drug-loaded platelet product or anti-cancer drug-loaded platelet derivatives and administering the products to a subject in need thereof. Surprisingly, it has been found that cryopreserved anti-cancer drug-loaded platelet product or anti-cancer drug-loaded platelet derivatives herein can retain at least 50% of the anti-cancer drug upon storing for at least 18 days. In some embodiments, cryopreserved anti-cancer drug-loaded platelet upon storing at a temperature in a range of −75° C. to −85° C. for at least 18 days can retain at least 50% of the anti-cancer drug. In some embodiments, anti-cancer drug-loaded platelet derivatives upon storing at a temperature in a range of 20° C. to 30° C. for at least 18 days can retain at least 50% of the anti-cancer drug. Accordingly, in an aspect, provided herein is a method for administering an anti-cancer drug to a subject, comprising: administering a therapeutically effective dose of anti-cancer drug-loaded platelet derivatives, or cryopreserved anti-cancer drug-loaded platelets, wherein the anti-cancer drug-loaded platelet derivatives, or the cryopreserved anti-cancer drug-loaded platelets are stored for at least 2 days before the administering.

In some embodiments, a therapeutically effective amount, or an effective amount of cryopreserved platelets, or cryopreserved anti-cancer drug-loaded platelets can be at least 0.25 unit, 0.5 unit, 0.75 unit, 1 unit, 2, or 3 units of thawed cryopreserved anti-cancer drug-loaded platelets or frozen anti-cancer drug-loaded platelets. For example, a therapeutically effective amount can be in the range of 0.25 unit to 5 units, in illustrative embodiments, 0.5 unit in a lower end and 3 units, 2 units, or 1 unit in a higher end, or 1 unit in a lower end and 4, 3, or 2 units in a higher end. In some embodiments, 1 unit corresponds to at least 1×10¹¹, 1.5×10¹¹, 2×10¹¹, or 2.5×10¹¹ platelets in a volume of at least around 40 ml, 45 ml, 50 ml, 55 ml, or 60 ml. In some embodiments, 1 unit corresponds to platelets in a range of 1×10¹¹ to 5×10¹¹, 1×10¹¹ to 4×10¹¹, 1×10¹¹ to 4×10¹¹, or 2×10¹¹ to 3×10¹¹ platelets in at least 40 or 50 ml. In illustrative embodiments, 1 unit corresponds to 2.5×10¹¹+/−4.2×10¹¹ platelets in 50+/−4 ml. The unit can refer to a unit of frozen platelets, frozen platelet derivatives, cryopreserved platelets or cryopreserved platelet derivatives, as will be understood depending on the context.

In some embodiments, a therapeutically effective amount, or an effective amount of anti-cancer drug-loaded platelet derivatives, or anti-cancer drug-loaded thrombosomes, cryopreserved platelets, or cryopreserved anti-cancer drug-loaded platelets can be at least 1.0×10⁷, 1.0×10⁸, or 1.0×10⁹ particles, such as anti-cancer drug-loaded platelet derivatives or cryopreserved anti-cancer drug-loaded platelets/kg of the subject. In some embodiments, a therapeutically effective amount, or an effective amount can be from 1.0×10⁷ on the low end of the range to 1.0×10¹⁰/kg, 1.0×10¹¹/kg or 1.0×10¹²/kg of the subject on the high end of the range. In some embodiments, a therapeutically effective amount, or an effective amount can be from 1.0×10⁷ to 1.0×10¹²/kg, 1.0×10⁷ to 1.0×10¹¹/kg, 1.5×10⁹ to 1.0×10¹²/kg, 1.6×10⁷ to 3.0×10⁹/kg, 1.5×10⁹ to 1.1×10¹⁰/kg, 3.0×10⁹ to 1.0×10¹²/kg, 3.0×10⁹ to 1.0×10¹¹/kg, or 3.0×10⁹ to 1.0×10¹⁰/kg of the subject. In some embodiments, a therapeutically effective amount, or an effective amount can be in a range of greater than 1.5×10⁹/kg of the subject on the low end of the range and 1.1×10¹², 1.0×10¹¹, 1.5×10¹⁰, 1.4×10¹⁰, 1.3×10¹⁰, 1.2×10¹⁰, or 1.1×10¹⁰/kg of the subject on the high end.

In some embodiments, administering herein can comprise administering one dose, or more than one dose of anti-cancer drug-loaded platelet derivatives, or anti-cancer drug-loaded thrombosomes, cryopreserved platelets, or cryopreserved anti-cancer drug-loaded platelets, for example at least 2, 3, 4, 5, 6, 7, 8, 9, or 10 doses in a subject. In some embodiments, each dose comprises an effective amount or therapeutically effective amount as disclosed herein. In some embodiments, administering herein can comprise administering one dose, or multiple doses, such as 2, 3, 4, or 5× amounts at the same time, or multiple times to a subject. For example, administering herein can comprise administering between 2 to 5× amounts provided herein, at least 2, 3, 4, 5, 6, 7, 8, 9, or 10 times to a subject. In some embodiments, administering multiple doses herein can be done within 24 hours, 20 hours, 18 hours, 15 hours, 12 hours, 8 hours, 6 hours, 4 hours, 3 hours, 1 hour, 30 minutes, 20 minutes, 15 minutes, 10 minutes or 5 minutes. In some embodiments, administering multiple doses can be done daily, weekly, monthly, and/or as needed depending on the overall health and/or tumor burden status of the subject.

In some embodiments, a subject herein can be a mammal. In some embodiments, a subject herein can be a human. In other embodiments, a subject herein can be a non-human mammal, or a non-human animal. In some embodiments, a non-limiting list of such a non-human animal can be canines, equines, and felines.

In some embodiments of any of the aspects and embodiments herein that include a composition, or in some compositions used in or formed by a process, the platelets or platelet derivatives are derived from a non-human animal. In some embodiments, the non-human animal is selected from the group consisting of canines, equines, and felines. In some exemplary embodiments, the platelet derivatives are derived from canines. In some embodiments, the platelets or platelet derivatives are derived from a human subject.

In some embodiments, the administering herein can include administering topically. Administering can include administering parenterally. Administering can include administering intravenously. Administering can include administering intramuscularly. Administering can include administering intrathecally. Administering can include administering subcutaneously. Administering can include administering intraperitoneally. In illustrative embodiments, administering herein includes administering an effective dose or a therapeutically effective dose of cryopreserved anti-cancer drug-loaded platelets or anti-cancer drug-loaded platelet derivatives intravenously.

Composition, process for preparing a composition, or a method for administering an anti-cancer agent herein can include anti-cancer drugs for treating a wide variety of cancers. Therefore, compositions, processes or methods herein can be used for treating a wide variety of cancers. Non-limiting examples of cancers can include breast cancer, liver cancer, ovarian cancer, lung cancer, renal cancer, bladder cancer, prostate cancer, pancreatic cancer, cervical cancer, color cancer, testicular cancer, thyroid cancer, bile duct cancer, esophageal cancer, skin cancer, kidney cancer, or endometrial cancer. Cancer can comprise carcinoma, myeloma, leukemia, lymphoma, or sarcoma. Cancer can comprise acute lymphoblastic leukemia (ALL), acute myeloid leukemia (AML), adrenocortical carcinoma, AIDS-related cancers, anal cancer, appendix cancer, brain cancer, bone cancer, bronchial cancer, cervical cancer, chronic lymphocytic leukemia (CLL), chronic myelogenous leukemia (CML), chronic myeloproliferative neoplasms, colorectal cancer (CLC), uterine cancer, esophageal cancer, eye cancer (e.g., intraocular melanoma, retinoblastoma, etc.), fallopian tube cancer, gallbladder cancer, gastrointestinal neuroendocrine tumors, germ cell tumors, testicular cancer, ovarian cancer, head cancer, neck cancer, lip and oral cavity cancer, metastatic cancers, neuroblastoma, non-small cell lung cancer (NSCLC), pancreatic cancer, penile cancer, parathyroid cancer, pharyngeal cancer, prostate cancer, pulmonary inflammatory myofibroblast tumor, rectal cancer, retinoblastoma, salivary gland cancer, small intestine cancer, stomach cancer, urethral cancer, vaginal cancer, or vulvar cancer.

A non-limiting examples of diseases (therapeutic indications) that may be treated with the drug-loaded platelets are Acute lymphoblastic leukemia (ALL), Acute myeloid leukemia (AML), Breast cancer (can also be used as an adjuvant therapy for metastasized breast cancer post-surgery), Gastric cancer, Hodgkin lymphoma, Neuroblastoma, Non-Hodgkin lymphoma, Ovarian cancer, Small cell lung cancer, Soft tissue and bone sarcomas, Thyroid cancer, Transitional cell bladder cancer, Wilms tumor, and cancer. A non-limiting list of cargo and therapeutic indications for cargo(s) to be loaded into platelets are doxorubicin, olaparib, and paclitaxel for the therapeutic indications of Acute lymphoblastic leukemia (ALL), Acute myeloid leukemia (AML), Breast cancer (can also be used as an adjuvant therapy for metastasized breast cancer post-surgery), Gastric cancer, Hodgkin lymphoma, Neuroblastoma, Non-Hodgkin lymphoma, Ovarian cancer, Small cell lung cancer, Soft tissue and bone sarcomas, Thyroid cancer, Transitional cell bladder cancer, Wilms tumor, and cancer.

### Methods for Testing Cryopreserved Platelets or Platelet Derivatives for the Ability to Deliver an Anti-Cancer Drug to a Cancer Cell

In an aspect, provided herein is a method for testing platelet derivatives or cryopreserved platelets for the ability to deliver an anti-cancer drug to a cancer cell, comprising contacting platelet derivatives or cryopreserved platelets as disclosed herein with a binding partner of a marker present on the surface of the platelet derivatives or the cryopreserved platelets as disclosed herein, followed by detecting a presence or an absence of an interaction between the binding partner of the marker and the platelet derivatives or the cryopreserved platelets, wherein the presence of an interaction indicates that the platelet derivatives or the cryopreserved platelets have the ability to deliver the anti-cancer drug to the cancer cell. In some embodiments, a marker can be selected from one or more of CD41 (GPIIb), CD61 (GPIIIa), CD62P (P-selectin), GPIb, C-type lectin-like receptor 2 (CLEC-2), P2Y1/P2Y12, α_{IIb}β₃ integrin, PAR receptor, integrin, GPVI, and GPIb-V-IX.

In an aspect, provide herein is a method for testing platelet derivatives or cryopreserved platelets for the ability to deliver an anti-cancer drug to a cancer cell, comprising contacting platelet derivatives or cryopreserved platelets as disclosed herein with a cancer cell expressing one or more of a tumor-associated marker, followed by detecting for a presence or an absence of an interaction between the cancer cell and the platelet derivatives or the cryopreserved platelets, wherein the presence of an interaction indicates that the platelet derivatives or the cryopreserved platelets have the ability to deliver the anti-cancer drug to the cancer cell. In some embodiments, the tumor-associated marker comprises one or more of podoplanin, galectin-3, and CD-44.

Methods for Loading Platelets with Anti-Cancer Drugs

In some embodiments, platelets are isolated prior to incubating or treating the platelets with a drug, in illustrative embodiments, an anti-cancer drug.

Accordingly, in some embodiments, a method for preparing anti-cancer drug-loaded platelets comprises:
a) isolating platelets, for example in a liquid medium;
and
b) incubating or treating the platelets with an anti-cancer drug and with a loading buffer comprising a salt, a base, a loading agent, and optionally ethanol, to form the anti-cancer drug-loaded platelets.

Accordingly, in some embodiments, a method for preparing anti-cancer drug-loaded platelets comprises:
a) isolating platelets, for example in a liquid medium;
b) treating the platelets with an anti-cancer drug to form a first composition; and
c) treating the first composition with a buffer comprising a salt, a base, a loading agent, and optionally at least one organic solvent to form the anti-cancer drug-loaded platelets.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof.

Accordingly, in some embodiments, the method for preparing anti-cancer drug-loaded platelets comprises:
isolating platelets, for example in a liquid medium;
treating the platelets with a buffer comprising a salt, a base, a loading agent, and optionally at least one organic solvent, to form a first composition; and
treating the first composition with an anti-cancer drug, to form the anti-cancer drug-loaded platelets.

In some embodiments, no solvent is used. Thus, in some embodiments, a method for preparing anti-cancer drug-loaded platelets comprises:
a) isolating platelets, for example in a liquid medium; and
a) treating the platelets with an anti-cancer drug and with a loading buffer comprising a salt, a base, and a loading agent, to form the anti-cancer drug-loaded platelets, wherein the method does not comprise treating the platelets with an organic solvent such as ethanol.

Thus, in some embodiments, a method for preparing anti-cancer drug-loaded platelets comprises:
a) isolating platelets, for example in a liquid medium;
b) treating the platelets with an anti-cancer drug to form a first composition; and
c) treating the first composition with a buffer comprising a salt, a base, and a loading agent, to form the anti-cancer drug-loaded platelets,
   wherein the method does not comprise treating the platelets with an organic solvent such as ethanol and the method does not comprise treating the first composition with an organic solvent such as ethanol.

Thus, in some embodiments, a method for preparing anti-cancer drug-loaded platelets comprises:
isolating platelets, for example in a liquid medium;
treating the platelets with a buffer comprising a salt, a base, and a loading agent, to form a first composition; and
treating the first composition with an anti-cancer drug, to form the anti-cancer drug-loaded platelets.
   wherein the method does not comprise treating the platelets with an organic solvent such as ethanol and the method does not comprise treating the first composition with an organic solvent such as ethanol.

In some embodiments, isolating platelets comprises isolating platelets from blood.

In some embodiments, platelets are donor-derived platelets. In some embodiments, platelets are obtained by a process that comprises an apheresis step.

In some embodiments, platelets are derived in vitro. In some embodiments, platelets are derived or prepared in a culture prior to treating the platelets with a drug. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, a method for preparing anti-cancer drug-loaded platelets comprises:
a) preparing platelets; and
b) treating the platelets with an anti-cancer drug and with a loading buffer comprising a salt, a base, a loading agent, and optionally at least one organic solvent, to form the drug-loaded platelets.

Accordingly, in some embodiments, a method for preparing anti-cancer drug-loaded platelets comprises:
a) preparing platelets;
b) treating the platelets with an anti-cancer drug to form a first composition; and
c) treating the first composition with a buffer comprising a salt, a base, a loading agent, and optionally at least one organic solvent, to form the anti-cancer drug-loaded platelets.

Accordingly, in some embodiments, a method for preparing anti-cancer drug-loaded platelets comprises:
a) preparing platelets;
b) treating the platelets with a buffer comprising a salt, a base, a loading agent, and optionally at least one organic solvent, to form a first composition; and
c) treating the first composition with an anti-cancer drug, to form the anti-cancer drug-loaded platelets.

In some embodiments, no solvent is used. Thus, in some embodiments, a method for preparing anti-cancer drug-loaded platelets comprises:
a) preparing platelets; and
b) treating the platelets with an anti-cancer drug and with a loading buffer comprising a salt, a base, and a loading agent, to form the anti-cancer drug-loaded platelets, wherein the method does not comprise treating the platelets with an organic solvent such as ethanol.

Thus, in some embodiments, a method for preparing anti-cancer drug-loaded platelets comprises:
a) preparing platelets;
b) treating the platelets with an anti-cancer drug to form a first composition; and
c) treating the first composition with a buffer comprising a salt, a base, and a loading agent, to form the anti-cancer drug-loaded platelets, wherein the method does not comprise treating the platelets with an organic solvent such as ethanol and the method does not comprise treating the first composition with an organic solvent such as ethanol.

Thus, in some embodiments, a method for preparing anti-cancer drug-loaded platelets comprises:
a) preparing platelets;
b) treating the platelets with a buffer comprising a salt, a base, and a loading agent, to form a first composition; and
c) treating the first composition with an anti-cancer drug, to form the anti-cancer drug-loaded platelets, wherein the method does not comprise treating the platelets with an organic solvent such as ethanol and the method does not comprise treating the first composition with an organic solvent such as ethanol.

In some embodiments, the loading agent is a saccharide. In some embodiments, the saccharide is a monosaccharide. In some embodiments, the saccharide is a disaccharide. In some embodiments, the saccharide is a non-reducing disaccharide. In some embodiments, the saccharide is sucrose, maltose, trehalose, glucose (e.g., dextrose), mannose, or xylose. In some embodiments, the loading agent is a starch.

In some embodiments, the loading agent is a carrier protein. In some embodiments, the carrier protein is albumin. In some embodiments, the carrier protein is bovine serum albumin (BSA).

In some embodiments, a drug, or an anti-cancer drug can be selected from the group consisting of one of the following:
i. a small molecule (that is, an organic compound having a molecular weight up to about 2 KDalton);
ii. a protein;
iii. an oligopeptide;
iv. a non-miRNA nucleic acid, a non-siRNA, and/or a non-mRNA (e.g., non-miRNA, DNA, other naturally or non-naturally occurring nucleic acids, including various modifications thereof); and
V. an aptamer.

In some embodiments of various methods described herein, platelets are loaded with one or more any of a variety of drugs, or anti-cancer drugs. In some embodiments, platelets are loaded with a small molecule. For example, platelets can be loaded with one or more of vemurafenib (ZELBORAF®), dabrafenib (TAFINLAR®), encorafenib (BRAFTOVI™), BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, LXH254, trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, CS3006, WX-554, PD98059, CI1040 (PD184352), hypothemycin, FRI-20 (ON-01060), VTX-Ile, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-oxozeaenol, 5-iodotubercidin, GDC0994, ONC201, buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, erucylphophocholine, erufosine, SR13668, OSU-A9, PH-316, PHT-427, PIT-1, DM-PIT-1, triciribine (triciribine phosphate monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, MLN0128, AZD-2014, CC-223, AZD2014, CC-115, everolimus (RAD001), temsirolimus (CCI-779), ridaforolimus (AP-23573), tipifarnib, BMS-214662, L778123, L744832, FTI-277, PRI-724, CWP232291, PNU74654, PKF115-584, PKF118-744, PKF118-310, PFK222-815, CGP 049090, ZTM000990, BC21, methyl 3-{[(4-methylphenyl) sulfonyl]amino}benzoate (MSAB), AV65, iCRT3, iCRT5, iCRT14, SM04554, LGK 974, XAV939, curcumin (e.g., Meriva®), DIF-1, genistein, NSC668036, FJ9, BML-286 (3289-8625), IWP, IWP-1, IWP-2, JW55, G007-LK, pyrvinium, foxy-5, Wnt-5a, ipafricept (OMP-54F28), SM04690, SM04755, nutlin-3a, IWR1, JW74, okadaic acid, SB239063, SB203580, adenosine diphosphate (hydroxymethyl) pyrrolidinediol (ADP-HPD), 2-[4-(4-fluorophenyl) piperazin-1-yl]-6-methylpyrimidin-4 (3H)-one, PJ34, J01-017a, IC261, PF670462, bosutinib (BOSULIF®), PHA665752, imatinib (GLEEVEC®), ICG-001, Rp-8-Br-CAMP, SDX-308, WNT974, CGX1321, ETC-1922159, AD-REIC/Dkk3, WIKI4, windorphen, NTRC 0066-0, CFI-402257, a (5,6-dihydro)pyrimido[4,5-e]indolizine, BOS172722, S63845, AZD5991, AMG 176, 483-LM, MIK665, TASIN-1 (Truncated APC Selective Inhibitor), osimertinib (AZD9291, merelectinib, TAGRISSO™), erlotinib (TARCEVA®), gefitinib (IRESSA®), neratinib (HKI-272, NERLYNX®), lapatinib (TYKERB®), vetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), AC0010MA EAI045, canertinib (CI-1033), poziotinib (NOV120101, HM781-36B), AV-412, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, GW572016, pyrotinib (SHR1258), palbociclib, ribociclib, abemaciclib, olaparib, veliparib, iniparib, rucaparib, CEP-9722, E7016, E7449, PRN1371, BLU9931, FIIN-4, H3B-6527, NVP-BGJ398, ARQ087, TAS-120, CH5183284, Debio 1347, INCB054828, JNJ-42756493 (erdafitinib), rogaratinib (BAY1163877), FIIN-2, LY2874455, lenvatinib (E7080), ponatinib (AP24534), regorafenib (BAY 73-4506), dovitinib (TKI258), lucitanib (E3810), cediranib (AZD2171), nintedanib (OFEV®, BIBF 1120), brivanib (BMS-540215), ASP5878, AZD4547, BGJ398 (infigratinib), E7090, HMPL-453, MAX-40279, XL999, orantinib (SU6668), pazopanib (VOTRIENT®), anlotinib, AL3818, PRIMA-1 (p53 reactivation induction of massive apoptosis-1), APR-246 (PRIMA-1MET), 2-sulfonylpyrimidines such as PK11007, pyrazoles such as PK7088, zinc metallochaperone-1 (ZMC1; NSC319726/ZMC 1), a thiosemicarbazone (e.g., COTI-2), CP-31398, STIMA-1 (SH Group-Targeting Compound That Induces Massive Apoptosis), MIRA-1 (NSC19630) and its analogs, e.g., MIRA-2 and MIRA-3, RITA (NSC652287), chetomin (CTM), stictic acid (NSC87511), p53R3, SCH529074, WR-1065, arsenic compounds, gambogic acid, spautin-1, YK-3-237, NSC59984, disulfiram (DSF), G418, RETRA (reactivate transcriptional activity), PD0166285, 17-AAG, geldanamycin, ganetespib, AUY922, IPI-504, vorinostat/SAHA, romidepsin/depsipeptide, HBI-8000, RG7112 (RO5045337), RO5503781, MI-773 (SAR405838), DS-3032b, AM-8553, AMG 232, MI-219, MI-713, MI-888, TDP521252, NSC279287, PXN822, ATSP-7041, spiroligomer, PK083, PK5174, PK5196, nutlin 3a, RG7388, Ro-2443, FTY-720, ceramide, OP449, vatalanib (PTK787/ZK222584), TKI-538, sunitinib (SU11248, SUTENT®), thalidomide, lenalidomide (REVLIMID®), axitinib (AG013736, INLYTAR), RXC0004, ETC-159, LGK974, WNT-C59, AZD8931, AST1306, CP724714, CUDC101, TAK285, AC480, DXL-702, E-75, PX-104.1, ZW25, CP-724714, irbinitinib (ARRY-380, ONT-380), TAS0728, AST-1306, AEE-788, perlitinib (EKB-569), PKI-166, D-69491, HKI-357, AC-480 (BMS-599626), RB-200 h, ARRY-334543 (ARRY-543, ASLAN001), CUDC-101, IDM-1, decitabine, cytosine arabinoside, ORY1001 (RG6016), GSK2879552, INCB059872, IMG7289, CC90011, MI1, MI2, MI3, Mi2-2 (MI-2-2), MI463, MI503, MIV-6R, EPZ004777, EPZ-5676, SGC0946, CN-SAH, SYC-522, SAH, SYC-534, MM-101, MM-102, MM-103, MM-401, WDR5-0101, WDR5-0102, WDR5-0103, OICR-9429, tivantinib (ARQ 197), golvatinib (E7050), cabozantinib (XL 184, BMS-907351), foretinib (GSK1363089), crizotinib (PF-02341066), MK-2461, BPI-9016M, TQ-B3139, MGCD265, MK-8033, capmatinib (INC280, INCB28060), tepotinib (MSC2156119J, EMD1214063), CE-35562, AMG-337, AMG-458, PHA-665725, PF-04217903, SU11274, PHA-665752, HS-10241, ARGX-111, glumetinib (SCC244), EMD 1204831, AZD6094 (savolitinib, volitinib, HMPL-504), PLB1001, ABT-700, AMG 208, INCB028060, AL2846, HTI-1066, PT2385, PT2977, 17 allylamino-17-demethoxygeldanamycin, eribulin (HALAVEN®, E389, ER-086526), ibrutinib (PCI-32675, Imbruvica®) (1-[(3R)-3-[4-amino-3-(4-phenoxyphenyl) pyrazolo[3,4-d]pyrimidin-1-yl]piperidin-1-yl]prop-2-en-1-one); AC0058 (AC0058TA); N-(3-((2-((3-fluoro-4-(4-methylpiperazin-1-yl)phenyl)amino)-7H-pyrrolo[2,3-d]pyrimidin-4-yl)oxy)phenyl) acrylamide; acalabrutinib (ACP-196, Calquence®, rINN) (4-[8-amino-3-[(2S)-1-but-2-ynoylpyrrolidin-2-yl]imidazo[1,5-alpyrazin-1-yl]-N-pyridin-2-ylbenzamide); zanubrutinib (BGB-3111) ((7R)-2-(4-phenoxyphenyl)-7-(1-prop-2-enoylpiperidin-4-yl)-1,5,6,7-tetrahydropyrazolo[1,5-a]pyrimidine-3-carboxamide); spebrutinib (AVL-292, 1202757-89-8, Cc-292) (N-[3-[5-fluoro-2-[4-(2-methoxyethoxy)anilino]pyrimidin-4-yl]amino]phenyl]prop-2-enamide); poseltinib (HM71224, LY3337641) (N-[3-[2-[4-(4-methylpiperazin-1-yl) anilino]furo[3,2-d]pyrimidin-4-yl]oxyphenyl]prop-2-enamide); evobrutinib (MSC 2364447, M-2951) (1-[4-[6-amino-5-(4-phenoxyphenyl)pyrimidin-4-yl]amino]methyl]piperidin-1-yl]prop-2-en-1-one); tirabrutinib (ONO-4059, GS-4059, ONO/GS-4059, ONO-WG-307) (1-[4-[6-amino-5-(4-phenoxyphenyl)pyrimidin-4-yl]amino]methyl]piperidin-1-yl]prop-2-en-1-one); vecabrutinib (SNS-062) ((3R,4S)-1-(6-amino-5-fluoropyrimidin-4-yl)-3-[(3R)-3-[3-chloro-5-(trifluoromethyl) anilino]-2-oxopiperidin-1-yl]piperidine-4-carboxamide); dasatinib (Sprycel®; BMS-354825) (N-(2-chloro-6-methylphenyl)-2-[[6-[4-(2-hydroxyethyl) piperazin-1-yl]-2-methylpyrimidin-4-yl]amino]-1,3-thiazole-5-carboxamide); PRN1008, PRN473, ABBV-105, CG′806, ARQ 531, BIIB068, AS871, CB1763, CB988, GDC-0853, RN486, GNE-504, GNE-309, BTK Max, CT-1530, CGI-1746, CGI-560, LFM A13, TP-0158, dtrmwxhs-12, CNX-774, entrectinib, nilotinib, 1-((3S,4R)-4-(3-fluorophenyl)-1-(2-methoxyethyl) pyrrolidin-3-yl)-3-(4-methyl-3-(2-methylpyrimidin-5-yl)-1-phenyl-1H-pyrazol-5-yl) urea, AG 879, AR-772, AR-786, AR-256, AR-618, AZ-23, AZ623, DS-6051, Gö 6976, GNF-5837, GTx-186, GW 441756, LOXO-101, LOXO-195, MGCD516, PLX7486, RXDX101, TPX-0005, TSR-011, venetoclax (ABT-199, RG7601, GDC-0199), navitoclax (ABT-263), ABT-737, TW-37, sabutoclax, obatoclax, BIX-01294 (BIX), UNC0638, A-366, UNC0642, DCG066, UNC0321, BRD 4770, UNC 0224, UNC 0646, UNC0631, BIX-01338, INNO-406, KX2-391, saracatinib, PP1, PP2, ruxolitinib, lestaurtinib (CEP-701), momelotinib (GS-0387, CYT-387), pacritinib (SB1518), fedratinib (SAR302503), BI2536, BI6727, GSK461364, amsacrine, azacitidine, busulfan, carboplatin, capecitabine, chlorambucil, cisplatin, cyclophosphamide, cytarabine, dacarbazine, daunorubicin, docetaxel, doxifluridine, doxorubicin, epirubicin, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, streptozocin, tafluposide, temozolomide, teniposide, tioguanine, topotecan, uramustine, valrubicin, vinblastine, vincristine, vindesine, and vinorelbine. In some embodiment, the anti-cancer drug is a rubicin (e.g., doxorubicin, epirubicin, daunorubicin, idarubicin, and valrubicin). In some embodiments, the anti-cancer drug is a cancer chemotherapy drug having one or more or, in illustrative embodiments, all of the following properties: is a small molecule, has a size less than 2 kDa, and does not bind microtubules. In some embodiments, the anti-cancer drug does not bind microtubules. In some embodiments, the anti-cancer drug binds to microtubules.

In some embodiments, an anti-cancer drug herein does not bind to and/or in some embodiments does not have any specificity or affinity to microtubules or in some non-limiting examples, any component(s) of platelets, for example, human platelets. Not to be limited by theory, in such embodiments an anti-cancer drug herein can be retained in the platelets after loading despite not binding, and/or not having any natural affinity or specificity to any component of platelets. In some embodiments, anti-cancer drug herein is not modified to impart the anti-cancer drug any binding and/or specificity or affinity to any component of the platelets. In some embodiments, anti-cancer drug herein can include modification for purposes other than imparting specificity to any components of platelets.

In some embodiments of various methods described herein, platelets are loaded with one or more any of a variety of drugs, or anti-cancer drugs. In some embodiments, platelets are loaded with a protein (e.g., an antibody or antibody conjugate). For example, platelets can be loaded with one or more of cetuximab (ERBITUX®), necitumumab (PORTRAZZA™, IMC-11F8), panitumumab (ABX-EGF, VECTIBIX®), matuzumab (EMD-7200), nimotuzumab (h-R3, BIOMAb EGFRR), zalutumab, MDX447, OTSA 101, OTSA101-DTPA-90Y, ABBV-399, depatuxizumab (humanized mAb 806, ABT-806), depatuxizumab mafodotin (ABT-414), SAIT301, Sym004, MAb-425, Modotuximab (TAB-H49), futuximab (992 DS), zalutumumab, Sym013, AMG 595, JNJ-61186372, LY3164530, IMGN289, KL-140, RO5083945, SCT200, CPGJ602, GP369, BAY1187982, FPA144 (bemarituzumab), bevacizumab (AVASTIN®), ranibizumab, trastuzumab (HERCEPTIN®), pertuzumab (PERJETA®), trastuzumab-dkst (OGIVRI®), ado-trastuzumab emtansine (KADCYLA®, T-DM1), Zemab, DS-8201a, MFGR1877S, B-701, rilotumumab (AMG102), ficlatuzumab (AV-299), FP-1039 (GSK230), TAK701, YYB101, onartuzumab (MetMAb), ipilimumab (YERVOY®), tremelimumab (CP-675,206), pembrolizumab (KEYTRUDA®), nivolumab (OPDIVO®), atezolizumab (TECENTRIQ®), avelumab (BAVENCIO®), durvalumab (IMFINZI™), BIOO-1, BIOO-2, and BIOO-3.

In some embodiments of various methods described herein, platelets are loaded with one or more any of a variety of drugs, or anti-cancer drugs. In some embodiments, platelets are loaded with an oligopeptide. For example, platelets can be loaded with one or more of RGD-SSL-Dox, LPD-PEG-NGR, PNC-2, PNC-7, RGD-PEG-Suc-PD0325901, VWCS, FWCS, p16, Bac-7-ELP-p21, Pen-ELP-p21, TAT-Bim, Poropeptide-Bax, R8-Bax, CT20p-NP, RRM-MV, RRM-IL 12, PNC-27, PNC-21, PNC-28, Tat-aHDM2, Int-H1-S6A, F8A, Pen-ELP-H1, BACI-ELP-H1, goserelin, leuprolide, Buserelin, Triptorelin, Degarelix, Pituitary adenylate cyclase activating peptide (PACAP), cilengitide, ATN-161 (AcPHSCN-NH2), TTK, LY6K, IMP-3, P16_37-63, VEGFR1-A24-1084, uMMP-2, uTIMP-1, MIC-1/GDF15, RGS6, LGR5, PGI/II, CA242, EN2, UCP2, a HER-2 peptide, MUCIm, HNP1-3, L-glutamine L-tryptophan (IM862), CPAA-783-EPPT1, serum C-peptide, WT1, KIF20A, GV1001, LY6K-177, PAP-114-128, E75, SU18, SU22, ANP, TCP-1, F56, WT1, TERT572Y, disruptin, TREM-1, LFC131, BPP, TH10, BC71, RC-3095, RC-3940-II, RC-3950, (KLAKLAK) 2, RGD-(KLAKLAK) 2, NGR-(KLAKLAK) 2, and SAH-8 (stapled peptides).

In some embodiments of various methods described herein, platelets are loaded with one or more any of a variety of drugs, or anti-cancer drugs. In some embodiments, platelets are loaded with a non-miRNA nucleic acid, a non-siRNA nucleic acid, and a non-mRNA nucleic acid (e.g., non-miRNA, DNA, other naturally or non-naturally occurring nucleic acids, and polymers thereof), including various modifications thereof). For example, platelets can be loaded with one or more of SPC2996, SIRNAPLUS, ALN-HTT, ISIS-199044, custirsen (OGX-011, ISI-112989, TV-1011), ISIS-AR-2.5RX (ISIS-ARRx, AZD-5312, ISIS-AZ1Rx, ISIS-560131), ISIS-STAT3-2.5Rx (ISIS-STAT3-2.5Rx, ISIS-481464, AZD-9150), BP-100-1.01, NOX-A12 (olaptesed peqol), PNT-2258, ATL-1103, RX-0201, ACT-GRO-777, litenimod, trabedersen (AP-2009), IMO-2055, OHR-118, imetelstat, GNKG-168, RG-6061, SPC-3042, STAT3 decoys, an anti-CD22 antibody-MXD3 antisense oligonucleotide conjugate, AST-008, ASncmtRNA, an EGFRAS GPNA, ASPH-1047 (ASPH-0047), STICKY SIRNA, aganirsen, BO-110, NOX—S93, Adva-R46, EZN-4482, EZN-4496, EZN-3889, EZN-3892, EZN-4150, IMO-2125 (HYB-2125), OGX-225, ATL-1101, aqatolimod, AGX-1053, AEG-35156, qataparsen, ISS-1018, CpG-1826 (ODN-1826), CpG-2216 (ODN-2216), CpG-2395, oblimersen, pbi-shRNA K-ras LP, LNA anti-miR-155, ISIS-20408, ISIS-199044, AP-11014, NOX-A50, beclanorsen, ISIS-345794, ISIS-15421, GRO-29A, LOR-2501 (GTI-2501), ISIS-7597, ISIS-3466, ISIS-2503, and GEM-231.

In some embodiments of various methods described herein, platelets are loaded with one or more any of a variety of drugs, or anti-cancer drugs. In some embodiments, platelets are loaded with an aptamer. For example, platelets can be loaded with one or more of ARC126 (RNA), AX102 (RNA), SL (2)-B (DNA), RNV66 (DNA), AS1411 (DNA), FCL-II (DNA, modified form AS1411), NOX-A12 (RNA), E0727 (RNA), CL428 (RNA), KDI130 (RNA), TuTu2231 (RNA), Trimeric apt (DNA), PNDA-3 (DNA), TTA140,41 (DNA), GBI-1042 (DNA), NAS-24 (DNA), YJ-1 (RNA), AGE-apt (DNA), A-P50 (RNA), GL21.T (RNA), OPN—R3 (RNA), AGC03 (DNA), cy-apt (DNA), BC15 (DNA), A9g (RNA), ESTA (DNA), M12-23 (RNA), OX40-apt (RNA), Del60 (RNA), PSMA-4-1BB-apt (RNA), CD16a/c-Met-apt (RNA), VEGF-4-1BB apt (DNA), MP7 (DNA), aptPD-L1 (DNA), R5A1 (RNA), CL-42 (RNA), CD44-EpCAM aptamer (RNA), TIM3Apt (RNA), CD40apt (RNA), AptCTLA-4 (DNA), AON-D211-Aptamer (RNA/DNA), and BN-210.

In some embodiments, a drug, or an ant-cancer drug loaded into platelets is modified. For example, a drug can be modified to increase its stability during the platelet loading process, while the drug is loaded into the platelet, and/or after the drug's release from a platelet. In some embodiments, the modified drug's stability is increased with little or no adverse effect on its activity. For example, the modified drug can have at least 70%, 75%, 80%, 85%, 90%, 95%, 96%, 97%, 98%, 99% or more of the activity of the corresponding unmodified drug. In some embodiments, the modified drug has 100% (or more) of the activity of the corresponding unmodified drug. Various modifications that stabilize drugs are known in the art. In some embodiments, the drug is a nucleic acid, which nucleic acid is stabilized by one or more of a stabilizing oligonucleotide (see, e.g., U.S. Application Publication No. 2018/0311176), a backbone/side chain modification (e.g., a 2-sugar modification such as a 2′-fluor, methoxy, or amine substitution, or a 2′-thio (—SH), 2′-azido (—N3), or 2′-hydroxymethyl (—CH2OH) modification), an unnatural nucleic acid substitution (e.g., an S-glycerol, cyclohexenyl, and/or threose nucleic acid substitution, an L-nucleic acid substitution, a locked nucleic acid (LNA) modification (e.g., the ribose moiety of an LNA nucleotide is modified with an extra bridge connecting the 2′ oxygen and 4′ carbon), conjugation with PEG, a nucleic acid bond modification or replacement (e.g., a phosphorothioate bond, a methylphosphonate bond, or a phosphorodiamidate bond), a reagent or reagents (e.g., intercalating agents such as coralyne, neomycin, and ellipticine; also see US Publication Application Nos. 2018/0312903 and 2017/0198335, each of which are incorporated herein by reference in their entireties, for further examples of stabilizing reagents). In some embodiments, the drug is a polypeptide, which polypeptide is stabilized by one or more of cyclization of the peptide sequence [e.g., between side chains or ends of the peptide sequence (for example, head to tail, N-backbone to N-backbone, end to N-backbone, end to side chain, side chain to N-backbone, side chain to side chain) through disulfide, lanthionine, dicarba, hydrazine, or lactam bridges], a backbone/side chain modification, an unnatural residue substitution (e.g., a D-amino acid, an N-methyl-α-amino acid, a non-proteogenic constrained amino acid or a β-amino acid), a peptide bond modification or replacement [e.g., NH-amide alkylation, the carbonyl function of the peptide bond can be replaced by CH2 (reduced bond:—CH2-NH—), C(═S) (endothiopeptide, —C(═S)—NH—) or PO2H (phosphonamide, —P(═O)OH—NH—), the NH-amide bond can be exchanged by O (depsipeptide, —CO—O—), S (thioester, —CO—S—) or CH2 (ketomethylene, —CO—CH2-), a retro-inverso bond (—NH—CO—), a methylene-oxy bond (—CH2-), a thiomethylene bond (—CH2-S—), a carba bond (—CH2-CH2-), and a hydroxyethylene bond (—CHOH-CH2-)], a disulfide-bridged conjugation with synthetic aromatics (see e.g., Chen et al. Org Biomol Chem. 2017, 15 (8): 1921-1929, which is incorporated by reference herein in its entirety), blocking N- or C-terminal ends of the peptide (e.g., by N-acylation, N-pyroglutamate, or C-amidation or the addition of carbohydrate chains through, for example, glycosylation with glucose, xylose, hexose), an N-terminal esterification (phosphoester), a pegylation modification, and a reagent or reagents (see, e.g., US Publication Application No. 2017/0198335). See. e.g., Vlieghe et al. Drug Discovery Today, 2010, 15, 40-56, which is incorporated by reference herein in its entirety. In some embodiments, a drug or an anti-cancer drug loaded into platelets is not modified.

In some embodiments, a drug, or an anti-cancer drug loaded into platelets is modified to include an imaging agent. For example, a drug can be modified with an imaging agent in order to image the drug loaded platelet in vivo. In some embodiments, a drug can be modified with two or more imaging agents (e.g., any two or more of the imaging agents described herein). In some embodiments, a drug loaded into platelets is modified with a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ⁵²Fe, ⁵⁵Co, ⁹⁴Tc or ⁶⁸Ga; or gamma-emitters such as ¹⁷¹Tc, ¹¹¹In, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to ¹²³I, ¹³¹I or ⁷⁷Br. For example, a positron-emitting radioactive non-metal can include, but is not limited to ¹¹C, ¹³N, ¹⁵O, ¹⁷F, ¹⁸F, ⁷⁵Br, ⁷⁶Br or ¹²⁴I. For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s (dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y, ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, a drug loaded into platelets can be associated with gold or other equivalent metal particles (such as nanoparticles). For example, a metal particle system can include, but is not limited to gold nanoparticles (e.g., Nanogold™).

In some embodiments, a drug, or an anti-cancer drug loaded into platelets that is modified with an imaging agent is imaged using an imaging unit. The imaging unit can be configured to image the drug loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen Z., et. al., Advance of Molecular Imaging Technology and Targeted Imaging Agent in Imaging and Therapy, Biomed Res Int, February 13, doi: 10.1155/2014/819324 (2014) have described various imaging techniques and which is incorporated by reference herein in its entirety.

In some embodiments, such as embodiments wherein the platelets are treated or incubated with the anti-cancer drug and the buffer sequentially as disclosed herein, the anti-cancer drug may be loaded in a liquid medium that may be modified to change the proportion of media components or to exchange components for similar products, or to add components necessary for a given application.

In some embodiments the loading buffer, and/or the liquid medium, may comprise one or more of a) water or a saline solution, b) one or more additional salts, or c) a base. In some embodiments, the loading buffer, and/or the liquid medium, may comprise one or more of a) DMSO, b) one or more salts, or c) a base.

In some embodiments the loading agent is loaded into the platelets in the presence of an aqueous medium. In some embodiments the loading agent is loaded in the presence of a medium comprising DMSO. As an example, one embodiment of the methods herein comprises treating platelets with an anti-cancer drug and with an aqueous loading buffer comprising a salt, a base, a loading agent, and optionally at least one organic solvent, to form the anti-cancer drug-loaded platelets. As an example, one embodiment of the methods herein comprises treating platelets with an anti-cancer drug and with a loading buffer comprising DMSO and comprising a salt, a base, a loading agent, and optionally ethanol, to form the anti-cancer drug-loaded platelets.

In some embodiments the loading buffer, and/or the liquid medium, may comprise one or more salts selected from phosphate salts, sodium salts, potassium salts, calcium salts, magnesium salts, and any other salt that can be found in blood or blood products, or that is known to be useful in drying platelets, or any combination of two or more of these.

Preferably, these salts are present in the composition at an amount that is about the same as is found in whole blood.

In some embodiments, the drug-loaded platelets are prepared by incubating the platelets with the anti-cancer drug in the liquid medium for different durations at or at different temperatures from 15-45° C., or about 37° C. (cell to drug volume ratio of 1:2).

In some embodiments, the platelets form a suspension in a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, one or more other components may be loaded in the platelets. In some embodiments, the one or more other components may be loaded concurrently with the drug. In some embodiments, the one or more other components and the drug may be loaded sequentially in either order. Components may include an agent (e.g., an anti-aggregation agent) that reduces or prevents platelet aggregation and activation during the loading process. Exemplary components (e.g., anti-aggregation agents) may include an anti-aggregation agent such as, Prostaglandin E1 or Prostacyclin and or EDTA/EGTA to prevent platelet aggregation and activation during the loading process. Additional non-limiting anti-aggregation agents may include, GR144053, FR171113, aspirin, MeSADP, PSB 0739, Cangrelor, Tirofiban (e.g., Aggrastat™), and MitoTEMPO, N-acetyyl-L-cysteine, cytcochalasin D, Staurosporine, Mepacrine, actezolamide, or dichloroacetate. These components may be used alone or in combination with one another.

Accordingly, in some embodiments, an agent suitable for treatment of cancer, for example, an anti-cancer drug, such as doxorubicin, may be loaded together with, prior to, or following, a GPIIb/IIIa inhibitor. In some embodiments, the cancer is acute lymphoblastic leukemia. In some embodiments, the cancer is acute myeloid leukemia. In some embodiments, the cancer is breast cancer or metastasized breast cancer. In some embodiments, the cancer is gastric cancer. In some embodiments, the cancer is Hodgkin lymphoma. In some embodiments, the cancer is neuroblastoma. In some embodiments, the cancer is Non-Hodgkin lymphoma. In some embodiments, the cancer is ovarian cancer. In some embodiments, the cancer is small cell lung cancer. In some embodiments, the cancer is soft tissue and bone sarcomas. In some embodiments, the cancer is thyroid cancer. In some embodiments, the cancer is transitional cell bladder cancer. In some embodiments, the cancer is Wilms tumor. In some embodiments, the cancer is liver cancer.

Accordingly, in some embodiments, an agent suitable for treatment of cancer, such as olaparib (also known as AZD-2281, MK-7339, trade name Lynparza®), may be loaded together with, prior to, or following, a GPIIb/IIIa inhibitor. In some embodiments, the cancer is ovarian cancer. In some embodiments, the cancer is breast cancer.

Accordingly, in some embodiments, an agent suitable for treatment of cancer, such as paclitaxel (Taxol®), may be loaded together with, prior to, or following, a GPIIb/IIIa inhibitor. In some embodiments, the cancer is Kaposi sarcoma. In some embodiments, the cancer is breast cancer. In some embodiments, the cancer is non-small cell lung cancer. In some embodiments, the cancer is ovarian cancer.

In some embodiments, an agent suitable for treatment of cancer, such as doxorubicin, may be loaded together with, prior to, or following, P2Y1 receptor activation inhibitor, a P2Y1 agonist, P2Y12 agonist, a P2Y13 agonist, a PAR 1 antagonist, a COX inhibitor, a P2Y12 inhibitor, a thiol supplement, a ROS antagonist, an actin polymerization inhibitor, protein kinase C inhibitor, phospholipase A2 inhibitor, Rho kinase inhibitor, a carbonic anhydrase inhibitor, or a PDK inhibitor.

In some embodiments, the one or more other components that are loaded in the platelets comprise Prostaglandin E1 (PGE1) or Prostacyclin.

In some embodiments, the one or more other components that are loaded in the platelets do not comprise Prostaglandin E1 or Prostacyclin.

In some embodiments, the one or more other components that are loaded in the platelets comprise EGTA.

In some embodiments, the one or more other components that are loaded in the platelets do not comprise EGTA.

In some embodiments, the one or more other components that are loaded in the platelets comprise EDTA.

In some embodiments, the one or more other components that are loaded in the platelets do not comprise EDTA.

The table below shows the effect of the addition of antiplatelet compounds on DOX-induced platelet aggregation:

TABLE A

| | | | | | | Max Platelet Count | | | |
|---|---|---|---|---|---|---|---|---|---|
| | | | | | | (Normalized to Untreated) | | | |
| | | | | | | 0.5 | 1 | 0.6 | 1.2 |
| | | | | Recommended | | mg/mL | mg/mL | mM | mM |
| # | Reagent | Target | Source | concentration | | DOX | DOX | DOX | DOX |
| 1 | GR144053 | GPIIb/IIIa | PMID: | IC50 = 37 nM | | 83% | 42% | N/A | N/A |
| | | inhibitor | 9700979 | | | | | | |
| 2 | PGE1 | P2Y1 | PMID: | 22 nM-1 μM | | N/A | N/A | N/A | N/A |
| | | receptor | 22385219 | | | | | | |
| | | activation | PMID: | | | | | | |
| | | Inhibitor | 22268418 | | | | | | |
| 3 | MeSADP | P2Y1, | PMID: | 10 | μM | 33% | 10% | N/A | N/A |
| | | P2Y12, and | 9442039 | | | | | | |
| | | P2Y13 | | | | | | | |
| | | Agonist | | | | | | | |
| 4 | FR171113 | PAR1 | PMID: | 0.3 | μM | 51% | 16% | N/A | N/A |
| | | antagonist | 10611442 | | | | | | |
| 5 | Aspirin (ASA) | COX | PMID: | 40-500 | μM | 52% | 15% | N/A | N/A |
| | | inhibitor | 3370916 | | | | | | |
| 6 | Cangrelor | P2Y12 | PMID: | 1 | μM | 47% | 26% | N/A | N/A |
| | | Inhibitor | 23236426 | | | | | | |
| 7 | PSB 0739 | P2Y12 | PMID: | 500 | nM | 26% | 18% | N/A | N/A |
| | | Inhibitor | 27695417 | | | | | | |
| 8 | N- | Thiol | PMID: | 5 | mM | N/A | N/A | 73% | N/A |
| | Acetylcysteine | Supplement | 19426282 page | | | | | | |
| | | | 1179 | | | | | | |
| 9 | MitoTEMPO | ROS | PMID: | 10 | μM | N/A | N/A | N/A | 37% |
| | | Antagonist | 25988386 | | | | | | |
| | | | methods 4.3 | | | | | | |
| 10 | Tirofiban | GPIIb/IIIa | PMID: | 5 | μM | N/A | N/A | 73% | N/A |
| | | inhibitor | 11406724 | | | | | | |
| | | | abstract | | | | | | |

The table below shows alternatively proposed antiplatelet compounds to combat DOX-induced platelet aggregation:

TABLE B

| | | | | Recommended | |
|---|---|---|---|---|---|
| # | Reagent | Target | Source | concentration | |
| 1 | Cytochalasin D | actin polymerization | PMID: 10682859 page 357 | 10 | μM |
| 2 | Staurosporine | protein kinase C | PMID: 10051374 methods | 25 nM to | |
| | | inhibitor | “PKC studies” | 10 μM | |
| | | | PMID: 11895774 (function | | |
| | | | or reagent) | | |
| 3 | Mepacrine | Phospholipase A2 | PMID: 3931692 | 2.5-20 | μM |
| | | inhibitor | | | |

In some embodiments, other components may include imaging agents. Such imaging agents can be loaded into platelet derivatives or cryopreserved platelets along with an anti-cancer drug herein. For example, an imaging agent can include, but is not limited to a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as 54Cu, 48V, 52Fe, 55Co, 94Tc or 68Ga; or gamma-emitters such as 171Tc, 111 In, 113 In, or 67Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to 123I, 131I or 77Br. For example, a positron-emitting radioactive non-metal can include, but is not limited to 11C, 13N, 15O, 17F, 18F, 75Br, 76Br or 124I. For example, a hyperpolarized NMR-active nucleus can include, but is not limited to 13C, 15N, 19F, 29Si and 31P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s (dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals 67Cu, 89Sr, 90Y, 153Sm, 185Re, 188Re or 192Ir, and non-metals 32P, 33P, 38S, 38Cl, 39Cl, 82Br and 83Br. In some embodiments, a drug loaded into platelets can be associated with gold or other equivalent metal particles (such as nanoparticles). For example, a metal particle system can include, but is not limited to gold nanoparticles (e.g., Nanogold™).

In some embodiments, the one or more imaging agents loaded concurrently with an anti-cancer drug is imaged using an imaging unit. The imaging unit can be configured to image the drug loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen Z. et. al., (2014) have described various imaging techniques and which is incorporated by reference herein in its entirety.

In some embodiments, the drug-loaded platelets are prepared by incubating the platelets with an anti-cancer drug in the liquid medium for different durations. The step of incubating the platelets to load one or more cargo, such as an anti-cancer drug(s), includes incubating the platelets for a time suitable for loading, as long as the time, taken in conjunction with the temperature, is sufficient for the anti-cancer drug to come into contact with the platelets and, preferably, be incorporated, at least to some extent, into the platelets. For example, in some embodiments, the drug-loaded platelets are prepared by incubating the platelets with the drug in the liquid medium for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the anti-cancer drug-loaded platelets are prepared by incubating the platelets with the drug in the liquid medium for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the anti-cancer drug-loaded platelets are prepared by incubating the platelets with the drug in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs, 10 minutes to 4 hours, 10 minutes to 3 hours, 10 minutes to 2.5 hours, 10 minutes to 2 hours, 10 minutes to 1.5 hours, 10 minutes to 1 hour, 15 minutes to 4 hours, 15 minutes to 3 hours, 15 minutes to 2.5 hours, 15 minutes to 2 hours, 15 minutes to 1.5 hours, 15 minutes to 1 hour, 15 minutes to 45 minutes, or 15 minutes to 35 minutes. In one embodiment, treating platelets, platelet derivatives, or thrombosomes with a an anti-cancer drug, a liquid medium, a buffer comprising a salt, a base, a loading agent, and optionally at least one organic solvent, and/or with any loading protocol described herein to form the drug-loaded platelets comprises contacting the platelets, platelet derivatives, or thrombosomes with a drug, a liquid medium, a buffer comprising a salt, a base, a loading agent, and optionally at least one organic solvent and/or with any loading protocol described herein for a period of time, such as a period of 5 minutes to 48 hours, 10 minutes to 6 hours, 10 minutes to 5 hours, 10 minutes to 4 hours, 10 minutes to 3 hours, 15 minutes to 3 hours, 30 minutes to 3 hours, 45 minutes to 3 hours, 1 hour to 3 hours, or 1.5 hours to 2.5 hours, or such as 2 hours.

In some embodiments, the anti-cancer drug-loaded platelets are prepared by incubating the platelets with the anti-cancer drug in the liquid medium at different temperatures. The step of incubating the platelets to load one or more cargo, such as anti-cancer drug(s), includes incubating the platelets with the anti-cancer drug in the liquid medium at a temperature that, when selected in conjunction with the amount of time allotted for loading, is suitable for loading. In general, the platelets with the drug in the liquid medium are incubated at a suitable temperature (e.g., a temperature above freezing) for at least a sufficient time for the drug to come into contact with the platelets. In embodiments, incubation is conducted at 37° C. In certain embodiments, incubation is performed at 4° C. to 45° C., such as 15° C. to 42° C. For example, in embodiments, incubation is performed at 35° C. to 40° C. (e.g., 37° C.) for 110 to 130 (e.g., 120) minutes and for as long as 24-48 hours.

In some embodiments of a method of preparing anti-cancer drug-loaded platelets disclosed herein, the method further comprises acidifying the platelets, or pooled platelets, to a pH of about 6.0 to about 7.4, prior to a treating step disclosed herein. In some embodiments, the method comprises acidifying the platelets to a pH of about 6.5 to about 6.9. In some embodiments, the method comprises acidifying the platelets to a pH of about 6.6 to about 6.8. In some embodiments, the acidifying comprises adding to the pooled platelets a solution comprising Acid Citrate Dextrose.

In some embodiments, the platelets are isolated prior to a treating step. In some embodiments, the method further comprises isolating platelets by using centrifugation. In some embodiments, the centrifugation occurs at a relative centrifugal force (RCF) of about 800 g to about 2000 g. In some embodiments, the centrifugation occurs at relative centrifugal force (RCF) of about 1300 g to about 1800 g. In some embodiments, the centrifugation occurs at relative centrifugal force (RCF) of about 1500 g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, the platelets are at a concentration from about 2,000 platelets/μl to about 500,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 200,000,000 platelets/μl.

In some embodiments, the buffer is a loading buffer comprising the components as listed in Table 1 herein. In some embodiments, the loading buffer comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the loading buffer includes from about 0.5 mM to about 100 mM of the one or more salts. In some embodiments, the loading buffer includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the loading buffer includes about 5 mM, about 75 mM, or about 80 mM of the one or more salts.

In some embodiments, the loading buffer includes one or more buffers, e.g., N-2-hydroxyethylpiperazine-N′-2-ethanesulfonic acid (HEPES), or sodium-bicarbonate (NaHCO₃). In some embodiments, the loading buffer includes from about 5 to about 100 mM of the one or more buffers. In some embodiments, the loading buffer includes from about 5 to about 50 mM (e.g., from about 5 mM to about 40 mM, from about 8 mM to about 30 mM, about 10 mM to about 25 mM) about of the one or more buffers. In some embodiments, the loading buffer includes about 10 mM, about 20 mM, about 25 mM, or about 30 mM of the one or more buffers. In some embodiments, loading buffer herein can include a buffer having a pH in the range of 6.0 to 8.0, 6.2 to 8.0, 6.5 to 8.0, 6.0 to 7.7, or 6.0 to 7.5.

In some embodiments, the loading buffer includes one or more saccharides, such as monosaccharides and disaccharides, including sucrose, maltose, trehalose, glucose, mannose, dextrose, and xylose. In some embodiments, the loading buffer includes from about 10 mM to about 1,000 mM of the one or more saccharides. In some embodiments, the loading buffer includes from about 50 to about 500 mM of the one or more saccharides. In embodiments, one or more saccharides is present in an amount of from 10 mM to 500 mM, 10 mM to 400 mM, 10 mM to 300 mM, 10 mM to 200 mM, 10 mM to 150 mM, 20 mM to 200 mM, 30 mM to 200 mM, 40 mM to 200 mM, 50 mM to 200 mM, 60 mM to 500 mM, 60 mM to 400 mM, 60 mM to 300 mM, 60 mM to 250 mM, 60 mM to 200 mM. In some embodiments, one or more saccharides comprises trehalose in a concentration of above 50 mM and below 500 mM, for example in a range of between 60 mM at a lower end to 100 mM, 200 mM, 300 mM, 400 mM, or 500 mM at a higher end. In some embodiments, one or more saccharides is present in an amount of from 50 mM to 200 mM. In embodiments, one or more saccharides is present in an amount from 100 mM to 150 mM.

In some embodiments, the loading buffer includes adding an organic solvent, such as ethanol, to the loading solution. In such a loading buffer, the solvent can range from about 0.1% (v/v) to about 5.0% (v/v), such as from about 0.3% (v/v) to about 3.0% (v/v), or from about 0.5% (v/v) to about 2% (v/v).

In some embodiments, the drug comprises doxorubicin (“DOX”). DOX interacts with DNA by intercalation and inhibits macromolecular biosynthesis (Tacar, O. et. al., Doxorubicin: an update on anticancer molecular action, toxicity and novel drug delivery systems, The Journal of Pharmacy and Pharmacology, 65 (2): 157-70. doi: 10.1111/j.2042-7158.2012.01567.x. PMID 23278683 (2013), which is incorporated herein by reference).

In some embodiment, the drug comprises paclitaxel. In some embodiment, the drug comprises paclitaxel that is not in the presence of Cremophor EL. In some embodiments, the drug comprises paclitaxel that is not in the presence (e.g., an excipient) of a polyexthoxylated castor oil. For example, paclitaxel that is not in the presence of an excipient comprising a polyethylene glycol ether.

In some embodiments, the drug comprises a poly ADP ribose polymerase (PARP) inhibitor (PARPi). PARPis prevent the normal repair of DNA breaks which in turn leads to cell death. In some embodiments, the PARPi is olaparib.

In some embodiments, the method further comprises incubating the anti-cancer drug in the presence of the loading buffer prior to the treatment step. In some embodiments, the method further comprises incubating the loading buffer and a solution comprising the anti-cancer drug and water at about 37° C. using different incubation periods. In some embodiments, the solution includes a concentration of about 1 nM to about 1000 mM of the anti-cancer drug. In some embodiments, the solution includes a concentration of about 10 nM to about 10 mM of the anti-cancer drug. In some embodiments, the solution includes a concentration of about 100 nM to 1 mM of the anti-cancer drug. In some embodiments, the solution includes a concentration of from about 10 mg/ml of water to about 100 mg/ml. In some embodiments, the solution includes a concentration of from about 20 mg/ml of water to about 80 mg/ml. In some embodiments, the solution includes a concentration of from about 40 mg/ml of water to about 60 mg/ml. In some embodiments, the incubation of the drug in the presence of the loading buffer is performed from about 1 minute to about 2 hours. In some embodiments, the incubation is performed at an incubation period of from about 5 minutes to about 1 hour. In some embodiments, the incubation is performed at an incubation period of from about 10 minutes to about 30 minutes. In some embodiments, the incubation is performed at an incubation period of about 20 minutes.

In some embodiments, the method further comprises mixing the platelets and the anti-cancer drug in the presence of the loading buffer at 37° C., using a platelet to anti-cancer drug volume ratio of 1:2. In some embodiments, the method further comprises incubating the platelets and the anti-cancer drug in the presence of the loading buffer at 37° C. using a platelet to anti-cancer drug volume ratio of 1:2, using different incubation periods. In some embodiments, the incubation is performed at an incubation period of from about 5 minutes to about 12 hours. In some embodiments, the incubation is performed at an incubation period of from about 10 minutes to about 6 hours. In some embodiments, the incubation is performed at an incubation period of from about 15 minutes to about 3 hours. In some embodiments, the incubation is performed at an incubation period of about 2 hours.

In some embodiments, the concentration of an anti-cancer drug in the anti-cancer drug-loaded platelets is from about 1 nM to about 1000 mM. In some embodiments, the concentration of an anti-cancer drug in the anti-cancer drug-loaded platelets is from about 10 nM to about 10 mM. In some embodiments, the concentration of an anti-cancer drug in the anti-cancer drug-loaded platelets is from about 100 nM to 1 mM.

In some embodiments, the method further comprises drying the anti-cancer drug-loaded platelets. In some embodiments, the drying step comprises freeze-drying the anti-cancer drug-loaded platelets. In some embodiments, the method further comprises rehydrating the anti-cancer drug-loaded platelets obtained from the drying step.

In some embodiments, anti-cancer drug-loaded platelets are prepared by using any one of the methods provided herein.

In some embodiments, rehydrated anti-cancer drug-loaded platelets are prepared by any one method comprising rehydrating the anti-cancer drug-loaded platelets provided herein.

The anti-cancer drug-loaded platelets may be then used, for example, for therapeutic applications as disclosed herein. As another example, the anti-cancer drug-loaded platelets may be employed in functional assays. In some embodiments, the anti-cancer drug-loaded platelets are cold stored, cryopreserved, or lyophilized (to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and 8,097,403. An exemplary spray-drying method includes: combining nitrogen, as a drying gas, with a loading buffer according to the present disclosure, then introducing the mixture into GEA Mobile Minor spray dryer from GEA Processing Engineering, Inc. (Columbia MD, USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE C
Exemplary Lyophilization Protocol

| | Step | Temp. Set | | Type | Duration | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the anti-cancer drug-loaded platelets that are obtained as disclosed herein, such as the step of freeze-drying the anti-cancer drug-loaded platelets that are obtained as disclosed herein, comprises incubating the platelets with a lyophilizing agent (e.g., a non-reducing disaccharide. Accordingly, in some embodiments, the methods for preparing anti-cancer drug-loaded platelets further comprise incubating the anti-cancer drug-loaded platelets with a lyophilizing agent

In some embodiments the lyophilizing agent is a saccharide. In some embodiments the saccharide is a disaccharide, such as a non-reducing disaccharide).

In some embodiments, the platelets are incubated with a lyophilizing agent for a sufficient amount of time and at a suitable temperature to load the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrrolidone (PVP), starch, and hydroxyethyl starch (HES). In some embodiments, exemplary lyophilizing agents can include a high molecular weight polymer, into the loading composition. By “high molecular weight” it is meant a polymer having an average molecular weight of about or above 70 kDa. Non-limiting examples are polymers of sucrose and epichlorohydrin. In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

In some embodiments, the process for preparing a composition includes adding an organic solvent, such as ethanol, to the loading solution. In such a loading solution, the solvent can range from 0.1% to 5.0% (v/v).

Within the process provided herein for making the compositions provided herein, addition of the lyophilizing agent can be the last step prior to drying. However, in some embodiments, the lyophilizing agent is added at the same time or before the drug, the cryoprotectant, or other components of the loading composition. In some embodiments, the lyophilizing agent is added to the loading solution, thoroughly mixed to form a drying solution, dispensed into a drying vessel (e.g., a glass or plastic serum vial, a lyophilization bag), and subjected to conditions that allow for drying of the solution to form a dried composition.

An exemplary saccharide for use in the compositions disclosed herein is trehalose. Regardless of the identity of the saccharide, it can be present in the composition in any suitable amount. For example, it can be present in an amount of 1 mM to 1 M. In some embodiments, it is present in an amount of from 10 mM 10 to 500 mM. In some embodiments, it is present in an amount of from 20 mM to 200 mM. In some embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

The step of incubating the platelets to load them with a cryoprotectant or as a lyophilizing agent includes incubating the platelets for a time suitable for loading, as long as the time, taken in conjunction with the temperature, is sufficient for the cryoprotectant or lyophilizing agent to come into contact with the platelets and, preferably, be incorporated, at least to some extent, into the platelets. In some embodiments, incubation is carried out for about 1 minute to about 180 minutes or longer.

The step of incubating the platelets to load them with a cryoprotectant or lyophilizing agent includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant or lyophilizing agent to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 mL/cm² (e.g., at least about 2.1 mL/cm², at least about 2.2 mL/cm², at least about 2.3 mL/cm², at least about 2.4 mL/cm², at least about 2.5 mL/cm², at least about 2.6 mL/cm², at least about 2.7 mL/cm², at least about 2.8 mL/cm², at least about 2.9 mL/cm², at least about 3.0 mL/cm², at least about 3.1 mL/cm², at least about 3.2 mL/cm², at least about 3.3 mL/cm², at least about 3.4 mL/cm², at least about 3.5 mL/cm², at least about 3.6 mL/cm², at least about 3.7 mL/cm², at least about 3.8 mL/cm², at least about 3.9 mL/cm², at least about 4.0 mL/cm², at least about 4.1 mL/cm², at least about 4.2 mL/cm², at least about 4.3 mL/cm², at least about 4.4 mL/cm², at least about 4.5 mL/cm², at least about 4.6 mL/cm², at least about 4.7 mL/cm², at least about 4.8 mL/cm², at least about 4.9 mL/cm², or at least about 5.0 mL/cm². In some embodiments, the SA/V ratio of the container can be at most about 10.0 mL/cm² (e.g., at most about 9.9 mL/cm², at most about 9.8 mL/cm², at most about 9.7 mL/cm², at most about 9.6 mL/cm², at most about 9.5 mL/cm², at most about 9.4 mL/cm², at most about 9.3 mL/cm², at most about 9.2 mL/cm², at most about 9.1 mL/cm², at most about 9.0 mL/cm², at most about 8.9 mL/cm², at most about 8.8 mL/cm², at most about 8.7 mL/cm², at most about 8.6, mL/cm² at most about 8.5 mL/cm², at most about 8.4 mL/cm², at most about 8.3 mL/cm², at most about 8.2 mL/cm², at most about 8.1 mL/cm², at most about 8.0 mL/cm², at most about 7.9 mL/cm², at most about 7.8 mL/cm², at most about 7.7 mL/cm², at most about 7.6 mL/cm², at most about 7.5 mL/cm², at most about 7.4 mL/cm², at most about 7.3 mL/cm², at most about 7.2 mL/cm², at most about 7.1 mL/cm², at most about 6.9 mL/cm², at most about 6.8 mL/cm², at most about 6.7 mL/cm², at most about 6.6 mL/cm², at most about 6.5 mL/cm², at most about 6.4 mL/cm², at most about 6.3 mL/cm², at most about 6.2 mL/cm², at most about 6.1 mL/cm², at most about 6.0 mL/cm², at most about 5.9 mL/cm², at most about 5.8 mL/cm², at most about 5.7 mL/cm², at most about 5.6 mL/cm², at most about 5.5 mL/cm², at most about 5.4 mL/cm², at most about 5.3 mL/cm², at most about 5.2 mL/cm², at most about 5.1 mL/cm², at most about 5.0 mL/cm², at most about 4.9 mL/cm², at most about 4.8 mL/cm², at most about 4.7 mL/cm², at most about 4.6 mL/cm², at most about 4.5 mL/cm², at most about 4.4 mL/cm², at most about 4.3 mL/cm², at most about 4.2 mL/cm², at most about 4.1 mL/cm², or at most about 4.0 mL/cm². In some embodiments, the SA/V ratio of the container can range from about 2.0 to about 10.0 mL/cm² (e.g., from about 2.1 mL/cm² to about 9.9 mL/cm², from about 2.2 mL/cm² to about 9.8 mL/cm², from about 2.3 mL/cm² to about 9.7 mL/cm², from about 2.4 mL/cm² to about 9.6 mL/cm², from about 2.5 mL/cm² to about 9.5 mL/cm², from about 2.6 mL/cm² to about 9.4 mL/cm², from about 2.7 mL/cm² to about 9.3 mL/cm², from about 2.8 mL/cm² to about 9.2 mL/cm², from about 2.9 mL/cm² to about 9.1 mL/cm², from about 3.0 mL/cm² to about 9.0 mL/cm², from about 3.1 mL/cm² to about 8.9 mL/cm², from about 3.2 mL/cm² to about 8.8 mL/cm², from about 3.3 mL/cm² to about 8.7 mL/cm², from about 3.4 mL/cm² to about 8.6 mL/cm², from about 3.5 mL/cm² to about 8.5 mL/cm², from about 3.6 mL/cm² to about 8.4 mL/cm², from about 3.7 mL/cm² to about 8.3 mL/cm², from about 3.8 mL/cm² to about 8.2 mL/cm², from about 3.9 mL/cm² to about 8.1 mL/cm², from about 4.0 mL/cm² to about 8.0 mL/cm², from about 4.1 mL/cm² to about 7.9 mL/cm², from about 4.2 mL/cm² to about 7.8 mL/cm², from about 4.3 mL/cm² to about 7.7 mL/cm², from about 4.4 mL/cm² to about 7.6 mL/cm², from about 4.5 mL/cm² to about 7.5 mL/cm², from about 4.6 mL/cm² to about 7.4 mL/cm², from about 4.7 mL/cm² to about 7.3 mL/cm², from about 4.8 mL/cm² to about 7.2 mL/cm², from about 4.9 mL/cm² to about 7.1 mL/cm², from about 5.0 mL/cm² to about 6.9 mL/cm², from about 5.1 mL/cm² to about 6.8 mL/cm², from about 5.2 mL/cm² to about 6.7 mL/cm², from about 5.3 mL/cm² to about 6.6 mL/cm², from about 5.4 mL/cm² to about 6.5 mL/cm², from about 5.5 mL/cm² to about 6.4 mL/cm², from about 5.6 mL/cm² to about 6.3 mL/cm², from about 5.7 mL/cm² to about 6.2 mL/cm², or from about 5.8 mL/cm² to about 6.1 mL/cm².

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments the lyophilizing agent as disclosed herein may be a high molecular weight polymer. By “high molecular weight” it is meant a polymer having an average molecular weight of about or above 70 kDa and up to 1,000,000 kDa. Non-limiting examples are polymers of sucrose and epichlorohydrin (polysucrose). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrrolidone (PVP), starch, and hydroxyethyl starch (HES).

In some embodiments, the loading buffer comprises an organic solvent, such as an alcohol (e.g., ethanol). In such a loading buffer, the amount of solvent can range from 0.1% to 5.0% (v/v).

In some embodiments the drug-loaded platelets prepared as disclosed herein have a storage stability that is at least about equal to that of the platelets prior to the loading of the drug.

The loading buffer may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino) propane-sulfonic acid (MOPS); phosphoric; and N-tris(hydroxymethyl)methyl-2-amminoethane sulfonic acid (TES).

Flow cytometry is used to obtain a relative quantification of loading efficiency by measuring the mean fluorescence intensity of the drug in the drug-loaded platelets. Platelets are evaluated for functionality by ADP and/or TRAP stimulation post-loading.

In some embodiments the drug-loaded platelets are lyophilized. In some embodiments the drug-loaded platelets are cryopreserved.

In some embodiments the drug-loaded platelets retain the loaded drug upon rehydration and release the drug upon stimulation by endogenous platelet activators.

In some embodiments the dried platelets (such as freeze-dried platelets) retain the loaded drug upon rehydration and release the drug upon stimulation by endogenous platelet activators. In some embodiments at least about 10%, such as at least about 20%, such as at least about 30% of the drug is retained. In some embodiments from about 10% to about 20%, such as from about 20% to about 30% of the drug is retained.

An example of a drug that may be loaded in a platelet is doxorubicin. Another example is of a drug that may be loaded in a platelet is olaparib. Another example of a drug that may loaded in a platelet is paclitaxel.

Various agents and/or procedures may be used to load the platelets with a drug. In some embodiments, the platelets are loaded with a liposomal formulation of the drug. In some embodiments, the drug is not comprised in a liposomal formulation. In some embodiments, the platelets are loaded with a drug previously incubated with a cell penetrating peptide. In some embodiments, the platelets are loaded with a drug previously incubated with a cationic lipid such as lipofectamine. In some embodiments, the platelets are loaded with the drug in the presence of a detergent. For example, the detergent may be saponin.

In some embodiments, the platelets are loaded by a process comprising endocytosis.

In some embodiments, the platelets are loaded by a process comprising electroporation.

In some embodiments, the platelets are loaded by a process comprising transduction.

In some embodiments, the platelets are loaded by a process comprising sonoporation.

In some embodiments, the platelets are loaded by a process comprising osmotic hypertonic/hypotonic loading/hypotonic shock. Hypotonic shock uses a solution with lower osmotic pressure to induce cell swelling leading to membrane permeability. Hypertonic shock may increase platelet loading of cryoprotectants or lyoprotectants (e.g., trehalose) (Zhou X, et. al., Loading Trehalose into Red Blood Cells by Improved Hypotonic Method, Cell Preservation Technology, 6 (2), https://doi.org/10.1089/cpt.2008.0001 (2008), which is herein incorporated by reference). Additionally and alternatively, hypotonic shock may allow the uptake and internalization of large and/or charged molecules through passive means, such as, endocytosis, micropinocytosis, and/or diffusion.

In some embodiments, the solutes in the hypertonic solution can be, in a non-limiting way, salts, low-molecular weight sugars (e.g., monosaccharides, disaccharides), or low molecular weight inert hydrophilic polymers.

In some embodiments, the platelets are loaded by a process comprising the use of transfection reagents (also described in WO2014118817A2, incorporated by reference herein in its entirety).

### Additional Considerations for Loading of Platelets

Exemplary protocols that employ the foregoing agents or procedures are described below:

A liposome is a vesicle made of phospholipid bilayer. This vesicle can be designed to encapsulate drug of interest, which is delivered inside a cell following the fusion of vesicle and cell membrane.

Liposome encapsulated Doxorubicin (chemotherapy drug) is prepared through rehydration of lyophilized lipids (Sigma-Aldrich, L4395-1VL) with drug in PBS followed by 30 seconds of agitation via vortex, then 30 minutes of incubation at 37° C. The liposomes are then incubated with platelets at 37° C. for 30 minutes. Cells are washed once via centrifugation to remove incorporated liposome encapsulated doxorubicin or free doxorubicin. Drug loaded platelets can be lyophilized in appropriate buffer to create Thrombosomes. Flow cytometry and fluorescence microscopy may be performed to assess drug loading and intracellular localization. A fluorescence microplate reader can be used to obtain quantification of drug load. Light transmission aggregometry will be used to evaluate platelet function post drug load.

Endocytosis is a process through which a cell takes in material from its surroundings. The cell invaginates its plasma membrane to wrap around fluid or particles in its immediate environment. The internalized vesicle buds off from the plasma membrane and remains inside the cell.

Co-incubation of platelets with drug of interest occurs at 37° C. for 1-4 hours during which drug is loaded into platelets via endocytosis. Loaded platelets may then be lyophilized to make Thrombosomes. Loaded drug is detected via flow cytometry or fluorescence microscopy, provided drug is fluorescently tagged or is itself fluorescent. Loaded drug can be detected by HPLC or a microplate reader (e.g., a Tecan plate reader). Endocytic inhibitors such as amiloride (1 mM), phenylarsine oxide (10 μM), cytochalasin D (4 μM), or dynasore (25 μM) can be used to confirm that platelet loading is achieved by endocytosis.

Pep1 is a 21 amino acid cell penetrating peptide with a C-terminal cysteamine group that shuttles cargo such as proteins or peptides into target cells. Pep1 consists of a hydrophobic domain linked to a hydrophilic domain. The hydrophobic, tryptophanrich domain can associate with a target cell membrane and the hydrophobic domains of the cargo protein (See e.g., Heitz, F, et. al., Twenty years of cell-penetrating peptides: from molecular mechanisms to therapeutics, British Journal of Pharmacology, 157, 195-206, (2009), which is incorporated herein by reference in its entirety).

The Pep1 and the cargo protein are complexed by co-incubation at 37° C. for 30 minutes. The Pep1: protein complex is incubated with platelets at 37° C. for at minimum 1 hour to allow Pep-1 mediated loading of protein cargo into the platelet. Platelets are washed by centrifugation to remove cellfree Pep1: protein complex. Loaded platelets may then be lyophilized to make Thrombosomes. Platelets that have accumulated Pep1 can be detected via flow cytometry or fluorescence microscopy if a fluorescent tag is attached to the Cterminus cysteamine of Pep1. If the cargo protein is fluorescently labeled, then platelets containing this cargo may also be detected using flow cytometry or fluorescence microscopy.

The HIV Tat protein is another example of a cell penetrating peptide. The Tat protein includes between 86 and 101 amino acids depending on the subtype. Tat is a regulatory protein that enhances the viral transcription efficiency. Tat also contains a protein transduction domain which functions as a cell-penetrating domain allowing Tat to cross cellular membranes.

Lipofectamine is a cationic lipid; the Lipofectamine positively charged head group interacts with the negatively charged phosphate backbone of nucleic acids to facilitate transfection. Cellular internalization of the nucleic acid is achieved by incubating cells with the complexed Lipofectamine and nucleic acid.

Prepare the Lipofectamine and nucleic acid complex in aqueous buffer at room temperature. Incubate the complexed Lipofectamine and nucleic acid with platelets for 2-3 hours. Transfected platelets may be lyophilized to create Thrombosomes. Fluorescently labeled nucleic acid can be detected via flow cytometry and visualized using fluorescence microscopy. This method of loading is applicable to both RNA and DNA.

An electroporation machine generates electrical pulses which facilitate formation of transient openings in plasma membranes. The increased plasma membrane permeability allows entry of large and/or charged cargo that would otherwise not enter the cell due to membrane barrier.

Perform electroporation of platelets in the presence of desired cargo. Cargos of interest can be detected by flow cytometry and fluorescence microscopy if they are fluorescently tagged.

The influx cell loading strategy harnesses osmosis to load cells with water soluble, polar compounds. Cells are initially placed in a hypertonic solution containing drug of interest. In this hypertonic solution, water will move out of the cell into solution while drug will move into the cell via pinocytosis. Following that, cells are placed in a hypotonic solution in which water will enter the cell, lysing the pinocytic vesicles and thereby releasing drug into the cytosol.

Incubate platelets in hypertonic loading medium containing drug compound at 37° C. for at least 1 hour. Isolate loaded platelets from solution via centrifugation, resuspend platelets in hypotonic lysis medium, and incubate at 37° C. Pinocytic vesicles will burst and release drug into the cytosol. Fluorescently labeled drug can be visualized using fluorescence microscopy to confirm internalization. Flow cytometry may be performed to quantify drug load per cell for fluorescent drug.

Viral vectors are commonly used for transduction of cells. The host cell is driven by the viral vector to express the protein of interest at high load.

Use lentiviral vector to transfect 293T cells to generate pseudovirus, which is collected from the supernatant of this cell culture. The pseudovirus is then used to transduce megakaryocytes. Inside the transduced megakaryocyte, viral core plasmid containing cytomegalovirus promotor drives overexpression of the protein of interest, which gets packaged into platelets that bud off from transduced megakaryocytes.

Human platelets express FcγRIIA receptor which binds to the Fc region of IgG and facilitates internalization of IgG immune complexes. This method of loading platelets provides a route for delivery of therapeutic antibodies.

Incubate fluorescently labeled IgG at 62° C. for 20 minutes to prepare IgG immune complexes. Incubate IgG immune complexes with platelets for 1 hour at 4° C. to allow cells to bind immune complexes. Next, incubate immune complex bound platelets at 37° C. to allow internalization of immune complexes. Flow cytometry can detect internalized fluorophore labeled IgG immune complexes. An anti-IgG-PE antibody specific for immune complexes can be used to identify surface bound, but not internalized, IgG-FITC immune complex.

Examples of drugs and of loading agents are as follows:

| | Cell penetrating | Osmotic hypertonic/ |
|---|---|---|
| Endocytosis | peptide | hypotonic loading |
| Dextran 10K | Dextran 10K | Dextran 10K |
| Dextran 500K | Dextran 3K | Dextran 3K |
| FITC-Albumin | Dextran 500K | Dextran 500K |
| FITC-Bovine IGG | FITC-albumin | FITC-albumin |
| FITC-F(ab)2 | FITC-Bovine IGG | FMLP |
| Histone H1 | FITC-F(ab)2 | Histone H1 |
| Lucifer yellow- | FMLP | Lucifer Yellow |
| slow uptake | | |
| PE | Histone H1 | PE |
| | | (PHYCOERYTRIN) |
| Rabbit IGG | Lucifer yellow | Rabbit IGG |
| Soybean Trypsin | PE | Soybean Trypsin |
| Inhibitor | | Inhibitor |
| Doxorubicin | Rabbit IGG | Doxorubicin |
| Olaparib | Soybean Trypsin Inhibitor | |
| Paclitaxel | | |

In some embodiments, the loading step comprises the use of dextran as a lyophilizing agent. In some embodiments the drug is an antibody. In some embodiments when the drug is an antibody, the drug is labeled with FITC (fluorescein isothiocyanate or 3′,6′-dihydroxy-6-isothiocyanatospiro [2-benzofuran-3,9′-xanthene]-1-one).

In some embodiments, drug-loaded platelets, drug-loaded platelet derivatives, or drug-loaded thrombosomes may shield the drug from exposure in circulation, thereby reducing or eliminating systemic toxicity (e.g. cardiotoxicity) associated with the drug. In some embodiments, drug-loaded platelets, drug-loaded platelet derivatives, or drug-loaded thrombosomes may also protect the drug from metabolic degradation or inactivation. In some embodiments, drug delivery with drug-loaded platelets, drug-loaded platelet derivatives, or drug-loaded thrombosomes may therefore be advantageous in treatment of diseases such as cancer, since drug-loaded platelets, drug-loaded platelet derivatives, or drug-loaded thrombosomes facilitate targeting of cancer cells while mitigating systemic side effects. In some embodiments, drug-loaded platelets, drug-loaded platelet derivatives, or drug-loaded thrombosomes may be used in any therapeutic setting in which expedited healing process is required or advantageous. In some embodiments, the therapeutic indications for cargo to be loaded into platelets include, for example, targeted depletion of cancer cells with chemotherapy drugs and therapeutic or prophylactic treatment of bacterial infection at site of injury with antibiotics.

In some embodiments, provided herein is a method of treating a disease as disclosed herein, comprising administering drug-loaded platelets, drug-loaded platelet derivatives, or drug-loaded thrombosomes as disclosed herein. In some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein.

In some embodiments, a drug may be fluorescent or labeled with a fluorescent moiety. For such a fluorescent or labeled drug, a correlation may be established between the fluorescence intensity and its concentration, and such a correlation may then be used to determine the concentration of the drug over a range of values. A non-limiting example of a method to calculate the doxorubicin amount can be according to the equation

$$X = ( Y + 344.92 ) / 996.6$$

where Y is the fluorescence and X is the concentration.
An analogous correlation can be derived for the value of the amount of doxorubicin in mg:

$$mg of doxorubicin = concentration ( µmol / L ) \times 543.42 g / mol \times 50 µl / well / {10}^{9} \\ or \\ mg of doxorubicin = ( \# µmol DOX / L ) ( mol / {10}^{6} µmol ) ( 543.52 g / mol ) ( {10}^{3} mg / g ) ( L / {10}^{3} ml ) ( 1 ml / {10}^{3} µl ) ( 50 µl / well )$$

An analogous correlation can be derived for the value of the amount of doxorubicin in mg/cell:

$$mg / cell = mg ( Intracellular + Membrane - bound doxorubicin ) /$$

$$total \# of cells in a well .$$

Thus, the concentration of doxorubicin may be quantified from its excitation/emission spectra.

Examples of loading buffer that may be used are shown in Tables 1-4:

TABLE 1
Loading Buffer
Table 1. Loading Buffer is used to load platelets via
endocytosis at 37° C. with gentle agitation as sample
is placed on a rocker. Adjust pH to 6.6-6.8

| | Concentration |
|---|---|
| | (mM unless |
| Component | otherwise specified) |
| NaCl | 75.0 |
| KCl | 4.8 |
| HEPES | 9.5 |
| NaHCO3 | 12.0 |
| Dextrose | 3 |
| Trehalose | 100 |
| Ethanol | 1% (v/v) |

TABLE 2
Buffer A
Table 2. Buffer A is used for loading platelets with
liposome encapsulated drug. Incubation done at 37° C.
with gentle agitation as sample is placed on a rocker.

| | Concentration |
|---|---|
| | (mM unless |
| Component | specified otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

TABLE 3
Buffer B
Table 3. Buffer B is used when incubating platelets with
fluorophore conjugated antibodies for flow cytometry.
This incubation is done at room temperature in the dark.

| | Concentration |
|---|---|
| | (mM unless |
| Component | otherwise specified) |
| Buffer and Salts | Table 4 (below) |
| BSA | 0.35% |
| Dextrose | 5 |
| pH | 7.4 |

Albumin is an optional component of Buffer B

TABLE 4
Concentration of HEPES and of Salts in Buffer B

| | Concentration |
|---|---|
| | (mM unless |
| Component | otherwise specified) |
| HEPES | 25 |
| NaCl | 119 |
| KCl | 5 |
| CaCl₂ | 2 |
| MgCl₂ | 2 |
| Glucose | 6 g/l |

In Table 4 the pH adjusted to 7.4 with NaOH

Albumin is an optional component of Buffer B

In some embodiments, drug-loaded platelets are prepared by incubating the platelets with the drug in a loading buffer having the components shown in the table below.

In some embodiments, the loading buffer has the components as listed above in Table 1.

In some embodiments, incubation is performed at 37° C. using a platelet to drug volume ratio of 1:2, using different incubation periods.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In an aspect, provided herein is a process for preparing a cryopreserved anti-cancer drug-loaded platelet composition comprising cryopreserved anti-cancer drug-loaded platelets, said process comprising:
a) incubating platelets with an anti-cancer drug in the presence of a loading buffer comprising a monosaccharide and/or a disaccharide to obtain a population of anti-cancer drug-loaded platelets;
b) freezing the population of anti-cancer drug-loaded platelets in a cryopreservation medium at a temperature of equal to or less than-50° C. to form an initial frozen anti-cancer drug-loaded platelet composition; and
c) storing the initial frozen anti-cancer drug-loaded platelet composition at a temperature of equal to or more than −40° C., but less than 0° C., or less than −5° C. for at least 2, 4, 5, 7, 8, or 10 days to form the cryopreserved anti-cancer drug-loaded platelet composition. In illustrative embodiments, incubating can be performed at a temperature in the range of 18-42° C., 18-40° C., 18-39° C., 20-42° C., 22-42° C., 24-42° C., 25-42° C., 30-42° C., or 35-42° C. In illustrative embodiment, incubating can be performed for a time period in the range of 5 minutes to 48 hours, 5 minutes to 24 hours, 5 minutes to 12 hours, 5 minutes to 10 hours, 5 minutes to 8 hours, 5 minutes to 6 hours, 5 minutes to 4 hours, 15 minutes to 6 hours, 15 minutes to 4 hours, 20 minutes to 4 hours, 20 minutes to 3 hours, or 20 minutes to 2.5 hours.

In an aspect, provided herein is a composition comprising frozen anti-cancer drug-loaded platelets in a cryopreservation medium in a frozen state, wherein the composition is capable of yielding one or more of the following recited properties after storage for 6 months, upon thawing:
a) is in a liquid state without requiring the addition of a liquid to achieve such liquid state;
b) exhibits a platelet count of at least 1.0×10¹¹/35 ml of the composition;
c) yields a single peak that corresponds to a compromised membrane peak in a membrane integrity assay;
d) exhibits a CD61-positive-microparticle content of less than 50% of the CD61 positive particles in the composition, and
e) retains at least 30%, 35%, 40%, 45%, 50%, 60%, or 70% of the anti-cancer drug. In some embodiments, the composition upon thawing generates thrombin in an in vitro thrombin generation assay.

In an aspect, provided herein, is a method for administering an anti-cancer drug to a subject, said method comprising:
obtaining a composition comprising cryopreserved or frozen anti-cancer drug-loaded platelets by the steps comprising:
   a) incubating platelets with an anti-cancer drug in the presence of a loading buffer comprising a monosaccharide and/or a disaccharide to obtain a population of anti-cancer drug-loaded platelets;
   b) freezing the population of anti-cancer drug-loaded platelets in a cryopreservation medium at a temperature of equal to or less than-50° C. to form an initial frozen anti-cancer drug-loaded platelet composition; and
   c) storing the initial frozen anti-cancer drug-loaded platelet composition at a temperature of equal to or more than −40° C., but less than 0° C., or less than −5° C. for at least 2, 4, 5, 7, 8, or 10 days to form the cryopreserved anti-cancer drug-loaded platelet composition comprising cryopreserved or frozen anti-cancer drug-loaded platelets,
administering a therapeutically effective dose of the composition comprising cryopreserved or frozen anti-cancer drug-loaded platelets to the subject.

In an aspect, provided herein, is a method for administering an anti-cancer drug to a subject, said method comprising:
administering a therapeutically effective dose of a composition comprising cryopreserved or frozen anti-cancer drug-loaded platelets to the subject,
wherein composition comprising frozen anti-cancer drug-loaded platelets in a cryopreservation medium in a frozen state, wherein the composition is capable of yielding one or more of the following recited properties after storage for 6 months, upon thawing:
   a) is in a liquid state without requiring the addition of a liquid to achieve such liquid state;
   b) exhibits a platelet count of at least 1.0×10¹¹/35 ml of the composition;
   c) yields a single peak that corresponds to a compromised membrane peak in a membrane integrity assay;
   d) exhibits a CD61-positive-microparticle content of less than 50% of the CD61 positive particles in the composition, and
   e) retains at least 30%, 35%, 40%, 45%, 50%, 60%, or 70% of the anti-cancer drug.

In an aspect, provided herein, is a method for administering an anti-cancer drug to a subject, comprising:
administering a therapeutically effective dose of cryopreserved anti-cancer drug-loaded platelets, wherein the cryopreserved anti-cancer drug-loaded platelets are stored for at least 2 days at a temperature of equal to or more than −40° C., but less than 0° C., or less than −5° C. before the administering.

In an aspect, provided herein, is a method for administering an anti-cancer drug to a subject, comprising:
a) preparing cryopreserved anti-cancer drug-loaded platelets by loading the anti-cancer drug into platelets and freezing the platelets to prepare the cryopreserved anti-cancer drug-loaded platelets, wherein the cryopreserved anti-cancer drug-loaded platelets comprise the anti-cancer drug;
b) storing cryopreserved anti-cancer drug-loaded platelets for at least 2 days at a temperature of equal to or more than −40° C., but less than 0° C., or less than −5° C.; and
c) administering a therapeutically effective dose of the anti-cancer drug-loaded platelet derivatives or the cryopreserved anti-cancer drug-loaded platelets to the subject.

In some aspects, there is provided a method for administering an anti-cancer drug to a subject, comprising:
administering a therapeutically effective dose of anti-cancer drug-loaded platelet derivatives, or cryopreserved anti-cancer drug-loaded platelets, wherein the subject has cancer, and the subject is other than a contraindicated person, wherein the contraindicated person has a stent, has a heart condition, had a diagnosed blood clot, and/or has an effective amount of an anti-platelet agent in their bloodstream.

In some aspects, there is provided a method for administering an anti-cancer drug to a subject, comprising:
administering a therapeutically effective dose of anti-cancer drug-loaded platelet derivatives, or cryopreserved anti-cancer drug-loaded platelets, wherein the anti-cancer drug-loaded platelet derivatives, or the cryopreserved anti-cancer drug-loaded platelets are stored for at least 2 days before the administering.

In some aspects, there is provided a method for administering an anti-cancer drug to a subject, comprising:
a) preparing anti-cancer drug loaded platelet derivatives, or cryopreserved anti-cancer drug-loaded platelets by loading the anti-cancer drug into platelets and lyophilizing or freezing the platelets to prepare the anti-cancer drug loaded platelet derivatives, or the cryopreserved anti-cancer drug-loaded platelets, respectively, wherein the anti-cancer drug loaded platelet derivatives, or the cryopreserved anti-cancer drug-loaded platelets comprise the anti-cancer drug;
b) storing the anti-cancer drug loaded freeze-dried platelet derivatives, or the cryopreserved anti-cancer drug-loaded platelets for at least 2 days; and
c) administering a therapeutically effective dose of the anti-cancer drug-loaded platelet derivatives or the cryopreserved anti-cancer drug-loaded platelets to the subject.

In some aspects, there is provided a method for preparing cryopreserved anti-cancer drug-loaded platelets, comprising:
a) incubating platelets with an anti-cancer drug in the presence of a loading buffer comprising a monosaccharide and/or a disaccharide at a temperature in the range of 18-42° C. for a time period in the range of 5 minutes to 24 hours, to obtain anti-cancer drug-loaded platelets;
b) cryopreserving the anti-cancer drug-loaded platelets in the presence of DMSO, glycerol, and polysucrose, to obtain cryopreserved anti-cancer drug-loaded platelets; and
c) storing the cryopreserved anti-cancer drug-loaded platelets for at least 2 days, wherein the cryopreserved anti-cancer drug-loaded platelets retain at least 5%, 10%, 15%, 20%, or 25% of the anti-cancer drug after the storing.

In some aspects, provided herein is a method for preparing anti-cancer drug-loaded platelet derivatives, comprising:
a) incubating platelets with an anti-cancer drug in the presence of a loading buffer comprising a monosaccharide and/or a disaccharide at a temperature in the range of 18-42° C. for a time period in the range of 5 minutes to 24 hours, to obtain anti-cancer drug-loaded platelets;
b) lyophilizing the anti-cancer drug-loaded platelets, to obtain anti-cancer drug loaded platelet derivatives; and
c) storing the anti-cancer drug-loaded platelet derivatives for at least 2 days, wherein the anti-cancer drug loaded platelet derivatives retain at least 5%, 10%, 15%, 20%, or 25% of the anti-cancer drug after the storing.

In some aspects, provided herein is a method for testing platelet derivatives or cryopreserved platelets for the ability to deliver an anti-cancer drug to a cancer cell, comprising:
a) obtaining platelet derivatives or cryopreserved platelets;
b) contacting the platelet derivatives or the cryopreserved platelets with a binding partner of a marker specific for platelets; and
c) detecting a presence or an absence of an interaction between the binding partner of the marker and the platelet derivatives or the cryopreserved platelets, wherein the presence of an interaction indicates that the platelet derivatives or the cryopreserved platelets have the ability to deliver the anti-cancer drug to the cancer cell.

In some aspects, provided herein is a method for testing platelet derivatives or cryopreserved platelets for the ability to deliver an anti-cancer drug to a cancer cell, comprising:
a) obtaining platelet derivatives or cryopreserved platelets;
b) contacting the platelet derivatives or the cryopreserved platelets with the cancer cell expressing one or more of a tumor-associated marker; and
c) detecting for a presence or an absence of an interaction between the cancer cell and the platelet derivatives or the cryopreserved platelets, wherein the presence of an interaction indicates that the platelet derivatives or the cryopreserved platelets have the ability to deliver the anti-cancer drug to the cancer cell.

In some aspects, provided herein is a composition comprising cryopreserved anti-cancer drug-loaded platelets herein for use in treating cancer in a subject.

In some aspects, provided herein is a composition comprising anti-cancer drug-loaded platelet derivatives herein for use in treating cancer in a subject.

In some aspects, provided herein is a composition comprising cryopreserved anti-cancer drug-loaded platelets herein for use in manufacturing a medicament for treating cancer in a subject.

In some aspects, provided herein is a composition comprising anti-cancer drug-loaded platelet derivatives herein for use for use in manufacturing a medicament for treating cancer in a subject.

In some embodiments, aspects herein can be combined with any aspects provided in claims herein.

In some aspects, provided herein is a method of preparing drug-loaded platelets, comprising:
treating platelets with a drug and with a loading buffer comprising a salt, a base, a loading agent, and optionally at least one organic solvent, to form the drug-loaded platelets.

In some aspects, provided herein is a method of preparing drug-loaded platelets, comprising:
providing platelets;
   and
treating the platelets with a drug and with a loading buffer comprising a salt, a base, a loading agent, and optionally at least one organic solvent
to form the drug-loaded platelets. In some embodiments, wherein the platelets are treated with the drug and with the buffer sequentially, in either order.

In some aspects, provided herein is a method of preparing drug-loaded platelets, comprising:
(1) treating platelets with a drug to form a first composition; and
(2) treating the first composition with a buffer comprising a salt, a base, a loading agent, and optionally at least one organic solvent, to form the drug-loaded platelets.

In some aspects, provided herein is a method of preparing drug-loaded platelets, comprising: treating the platelets with a buffer comprising a salt, a base, a loading agent, and optionally at least one organic solvent to form a first composition; and treating the first composition with a drug, to form the drug-loaded platelets.

In some aspects, provided herein is a method of preparing drug-loaded platelets, comprising: treating the platelets with a drug in the presence of a buffer comprising a salt, a base, a loading agent, and optionally at least one organic solvent to form the drug-loaded platelets.

In some embodiments of any of the aspects or embodiments herein that include a process for preparing cryopreserved anti-cancer drug-loaded platelets or cryopreserved anti-cancer drug-loaded platelet composition that include a transition in freezing and storing temperature, a process for preparing cryopreserved anti-cancer drug-loaded platelets or cryopreserved anti-cancer drug-loaded platelet composition, freezing can comprise freezing a population of anti-cancer drug-loaded platelets in a cryopreservation medium herein at a temperature of equal to or less than −50° C., −55° C., −60° C., or −65° C., to form an initial frozen anti-cancer drug-loaded platelet composition, and storing can comprise storing the initial frozen anti-cancer drug-loaded platelet composition at a temperature equal to or more than −45° C., −40° C., −35° C., −30° C., −25° C., or −20° C. for at least 2, 4, 6, 7, 10, 15, 20, or 25 days, 1 month, 2, 4, 6, 8, 10, or 12 months, 1 year, 2, 4, 6, 8, or 10 years, to form the cryopreserved anti-cancer drug-loaded platelet composition. In some embodiments, cryopreserved anti-cancer drug-loaded platelets herein are platelet derivatives. In illustrative embodiments, storing comprises storing the initial frozen anti-cancer drug-loaded platelet composition in a freezer set at a temperature of −20° C.+/−2° C. In some embodiments, the freezing comprises subjecting the population of anti-cancer drug-loaded platelets in the cryopreservation medium as disclosed herein at the temperature for at least 30 minutes, 1 hour, 2 hours, 3 hours, 4 hours, 8 hours, 12 hours, 1 day, 2 days, 7 days, 1 week, 2 weeks, 1 month, 2 months, 3 months, or 6 months to form the initial frozen anti-cancer drug-loaded platelet composition. For example, freezing can be done for a time period of 24 hours to 6 months, 24 hours to 5 months, 24 hours to 4 months, 1 day to 6 months, 1 day to 5 months, 1 day to 4 months. In some embodiments, the process further comprises thawing the cryopreserved anti-cancer drug-loaded platelets to form a liquid platelet anti-cancer drug-loaded composition, and administering an effective amount of the liquid platelet anti-cancer drug-loaded composition to a subject in need thereof. In some embodiments, the storing of an initial frozen anti-cancer drug-loaded platelet composition can be done for a time period in the range of 1 month to 10 years, 1 month to 8 years, 1 month to 7 years, 1 month to 5 years, 1 month to 3 years, or 1 month to 1 year. In some embodiments, the cryopreservation medium comprises dimethyl sulfoxide (DMSO) in a concentration in the range of 5% to 8%, for example, DMSO can be 6%+/−1%, +/0.8%, +/−0.6%, +/−0.5%, +/−0.4%, +/−0.3%, +/−0.2%, +/−0.1%. In some embodiments, the cryopreservation medium comprises DMSO in a concentration range of 0.5% to 8%, 2% to 8%, 3% to 8%, or 5% to 8%. In some embodiments, the initial frozen anti-cancer drug-loaded platelet composition is stored at the temperature of in the range of −10° C. to −30° C. for at least 1 month, 2, 3, 4, 5, or 6 months to form cryopreserved anti-cancer drug-loaded platelets, and the cryopreserved anti-cancer drug-loaded platelets upon thawing exhibits one or more of the following: i) retains at least 25%, 30%, 35%, 40%, 45%, or 50% of the anti-cancer drug upon thawing; ii) retains the ability to reduce bleeding in a subject in need thereof; iii) exhibit a platelet count recovery of at least 65%, 70%, or 75%; iv) exhibits a pH of equal to or more than 6.2; v) exhibit an aggregate-free swirling upon a visual inspection; vi) exhibits a platelet count of equal to or more than 1.7×10¹¹ platelets/bag or platelets/cryo-vessel, or per 20-35 ml. In some embodiments, the cryopreserved anti-cancer drug-loaded platelets upon thawing have a diameter in the range of 0.5-5 μm, 1-4 μm, 1-3 μm, or 0.5-2.5 μm, and wherein the composition upon thawing has a CD61-positive-microparticle content in the range of 10-30%, 10-35%, 10-40%, 10-45%, or 10-50%. In some embodiments, a therapeutically effective amount of cryopreserved anti-cancer drug-loaded platelets or cryopreserved anti-cancer drug-loaded platelet derivatives, frozen anti-cancer drug-loaded platelets or frozen anti-cancer drug-loaded platelet derivatives upon storing at a temperature equal to or more than −45° C., −40° C., −35° C., −30° C., −25° C., or −20° C. for at least 2, 4, 6, 7, 10, 15, 20, or 25 days, 1 month, 2, 4, 6, 8, 10, or 12 months, 1 year, 2, 4, 6, 8, or 10 years, upon thawing retain the ability to kill cancer cells or tumor cells in a subject in need thereof. In some embodiments, a therapeutically effective amount is an amount that is capable of killing cancer cells in a subject. Accordingly, in some embodiments, a therapeutically effective amount can be at least 0.25 unit, 0.5 unit, 0.75 unit, 1 unit, 2, or 3 units of thawed cryopreserved anti-cancer drug-loaded platelets or frozen anti-cancer drug-loaded platelets. For example, a therapeutically effective amount can be in the range of 0.25 unit to 5 units, in illustrative embodiments, 0.5 unit in a lower end and 3 units, 2 units, or 1 unit in a higher end, or 1 unit in a lower end and 4, 3, or 2 units in a higher end. In some embodiments, 1 unit corresponds to at least 1×10¹¹, 1.5×10¹¹, 2×10¹¹, or 2.5×10¹¹ platelets in a volume of at least around 40 ml, 45 ml, 50 ml, 55 ml, or 60 ml. In some embodiments, 1 unit corresponds to platelets in a range of 1×10¹¹ to 5×10¹¹, 1×10¹¹ to 4×10¹¹, 1×10¹¹ to 4×10¹¹, or 2×10¹¹ to 3×10¹¹ platelets in at least 40 or 50 ml. In illustrative embodiments, 1 unit corresponds to 2.5×10¹¹+/−4.2×10¹¹ platelets in 50+/−4 ml. The unit can refer to a unit of frozen platelets, frozen platelet derivatives, cryopreserved platelets or cryopreserved platelet derivatives, as will be understood depending on the context.

In some embodiments of any of the aspects or embodiments herein that include a composition comprising frozen anti-cancer drug-loaded platelets or cryopreserved anti-cancer drug-loaded platelets in a cryopreservation medium in a frozen state as disclosed herein, or a process for preparing a cryopreserved platelet composition, the composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., or −20° C.+/−5° C. upon thawing exhibits a platelet count of at least 1.0×10¹¹/20-35 ml of the composition, upon thawing retains the ability to retain at least 50% of the anti-cancer drug, and upon thawing retains the ability to kill cancer cells in a subject. For example, exhibits a platelet count of at least 1.0×10¹¹/20 ml of the composition, 1.0×10¹¹/25 ml of the composition, 1.0×10¹¹/30 ml of the composition, 1.0×10¹¹/35 ml of the composition. In some embodiments, a composition herein upon thawing exhibits a platelet count of at least 1.1×10¹¹/20-35 ml of the composition, 1.2×10¹¹/20-35 ml of the composition, 1.3×10¹¹/20-35 ml of the composition, 1.4×10¹¹/20-35 ml of the composition, 1.5×10¹¹/20-35 ml of the composition, 1.6×10¹¹/20-35 ml of the composition, or 1.7×10¹¹/20-35 ml of the composition. In some embodiments, the cryopreserved anti-cancer drug-loaded platelets herein are cryopreserved anti-cancer drug-loaded platelet derivatives. In some embodiments, the frozen anti-cancer drug-loaded platelets herein are frozen anti-cancer drug-loaded platelet derivatives. Typically, a composition as disclosed herein upon thawing is in a liquid state without requiring the addition of a liquid to achieve such a liquid state. In some embodiments, cryopreserved platelets, or frozen platelets herein do not comprise freeze-dried platelet derivatives or lyophilized platelet derivatives. In some embodiments, a composition upon thawing yields a single peak that corresponds to a compromised membrane peak in a membrane integrity assay. In some embodiments, the membrane integrity assay comprises incubating the composition with calcein acetoxymethyl (AM) to form a treated composition and analyzing the treated composition using flow cytometry. In some embodiments, the membrane integrity assay comprises incubating the composition with calcein acetoxymethyl (AM) for 20 minutes before performing the flow cytometry, and wherein the flow cytometry comprises detecting fluorescence produced by metabolized calcein AM and retained by particles in the treated composition. In some embodiments, the in vitro thrombin generation assay comprises generating thrombin in the presence of tissue factor, and phospholipids. In some embodiments, the in vitro thrombin generation assay consists of generating thrombin in the presence of tissue factor and phospholipids. In some embodiments, the frozen anti-cancer drug-loaded platelets or cryopreserved anti-cancer drug-loaded platelets herein upon thawing have a diameter in the range of 0.5-5 μm, 1-4 μm, 1-3 μm, or 0.5-2.5 μm. In some embodiments, a composition comprising frozen anti-cancer drug-loaded platelets herein upon thawing has a CD61-positive-microparticle content in the range of 5-50%, 5-45%, 5-40%, 5-35%, 5-30%, 10-30%, 10-40%, 10-50%, or 10-60%. In some embodiments, a composition as disclosed herein comprise CD61-positive-microparticles having a diameter less than 1 μm, 0.8 μm or 0.5 μm. In some embodiments, a composition herein upon thawing has a pH equal to or more than 6.0, 6.2, or 6.5. In some embodiments, a composition herein upon thawing has a pH in the range of 6.2 to 8.0. In some embodiments, a composition herein have a dispersed property such that after thawing the composition, the frozen anti-cancer drug-loaded platelets therein form no visible aggregates upon a visual inspection. In some embodiments, the composition comprises a dispersed suspension property such that aggregation of platelets in the composition is not observed by visual inspection of the composition after thawing. In some embodiments, the composition has a swirling property, such that swirling of the composition can be observed by visual inspection of the composition after thawing. In some embodiments, a composition comprises a cryopreservation medium comprises dimethyl sulfoxide (DMSO) at a concentration of 0.5% to 10%, 1% to 8%, or 3% to 8%. In some embodiments, in a composition herein, no more than 50% of the frozen platelet derivates upon thawing are positive for CD62, or Annexin V. For example, in a composition herein, particles positive for CD62 are less than 90%, 85%, 80%, 70%, 65%, 60%, or 50%. For example, in a composition herein, particles positive for Annexin V are less than 90%, 85%, 80%, 70%, 65%, 60%, or 50%. For example, in a composition herein, particles positive for Annexin V are in a range of 1-50%, 1-40%, 1-30%, 1-20%, 1-10%, 5-50%, 5-40%, 5-30%, or 5-20%. For example, in a composition herein, particles positive for CD62 are in a range of 1-50%, 1-40%, 1-30%, 1-20%, 1-10%, 5-50%, 5-40%, 5-30%, or 5-20%. In some embodiments, frozen anti-cancer drug-loaded platelets or cryopreserved anti-cancer drug-loaded platelets in a composition herein are less activated as compared to freeze-dried platelet derivatives or lyophilized platelet derivatives. In some embodiments, frozen anti-cancer drug-loaded platelets or cryopreserved anti-cancer drug-loaded platelets in a composition are not lyophilized platelet derivatives, or not freeze-dried platelet derivatives. In some embodiments, a composition herein exhibits a platelet count of at least 1.0×10¹¹/20 ml of the composition. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing exhibits a CD61-positive-microparticle content of less than 50% of the CD61 positive particles in the composition. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing yields a single peak that corresponds to a compromised membrane peak in a membrane integrity assay. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing generates thrombin in an in vitro thrombin generation assay. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing retains the ability to kill cancer cells or tumor cells in a subject, ability to reduce tumor burden in a subject, or the ability to reduce the weight of the tumor in a subject. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing retains at least 30%, 35%, 40%, 45%, 50%, 60%, or 70% of the loaded anti-cancer drug. In illustrative embodiments, a composition herein is capable of yielding one or more, two or more, three or more, or all of the properties after storing for 12 months. In some embodiments, a composition comprising frozen platelets in a cryopreservation medium in a frozen state, after storage for at least 1 month, 2, 3, 4, 5, 6, 8, 10, or 12 months, in an illustrative embodiments at a temperature in a range of −10° C. to −30° C., or −20° C.+/−5° C., upon thawing exhibits a liquid state without the addition of a liquid, is capable of killing cancer cells in a subject, exhibits a platelet count of at least 1.0×10^{11/35} ml of the composition, exhibits a CD61-positive-microparticle content of less than 50% of the CD61 positive particles in the composition, and/or generates thrombin in an in vitro thrombin generation assay.

In some embodiments of any aspects or embodiments herein that include cryopreserved anti-cancer drug-loaded platelets, a method for preparing cryopreserved anti-cancer drug-loaded platelets, a method for administering an anti-cancer agent to a subject, or cryopreserved anti-cancer drug-loaded platelets used in or obtained by any of the methods herein, an effective dose of cryopreserved anti-cancer drug-loaded platelets comprises the anti-cancer drug (e.g., a rubicin such as doxorubicin) that is at least 1.2, 1.3, 1.4, or 1.5 fold lower in amount and/or concentration than a regulatory agency-approved dose of the anti-cancer drug that is present in a liposomal formulation. In some embodiments, an effective dose of the anti-cancer drug in the cryopreserved anti-cancer drug-loaded platelets comprises the anti-cancer drug that is 1.5 to 10, 1.5 to 9, 1.5 to 8, 1.5 to 6, 1.5 to 5, 1.5 to 4, 1.5 to 3, 2 to 10, 3, to 10, 4 to 10, or 5 to 10 fold lower in amount and/or concentration than the dose of the anti-cancer drug that is present in a liposomal formulation of the anti-cancer drug that is administered to the subject. In some embodiments, the anti-cancer drug can be any drug disclosed herein, in some embodiments, the anti-cancer drug can be a rubicin selected from the group of doxorubicin, epirubicin, daunorubicin, idarubicin, or valrubicin, and in illustrative embodiments, the anti-cancer drug comprises doxorubicin, or any salts thereof. In some embodiments, an effective dose of the anti-cancer drug in the cryopreserved anti-cancer drug loaded platelets more effectively reduces a tumor burden, kills cancer or tumor cells in the subject, or reduces size of a tumor in the subject as compared to a dose of the anti-cancer drug that is identical or more than that of the anti-cancer drug in the cryopreserved anti-cancer drug loaded platelets, but present in a liposomal formulation that is administered to the subject. In some embodiments, the reduction in tumor burden, killing of cancer or tumor cells, or reduction in the size of a tumor can be at least 1.2, 1.4, 1.5, 1.75, 2, 3, 4, or 5 fold effective, or between 1.2 to 10, 1.5 to 10, 1.2 to 8, 1.2 to 6, 1.2 to 5, 1.2 to 4, 1.2 to 3, 1.5 to 10, 2 to 10, 3 to 10, or 5 to 10 fold effective in an effective dose of the anti-cancer drug in the cryopreserved anti-cancer drug loaded platelets as compared to a liposomal formulation of the anti-cancer drug. In some embodiments, an effective dose of the anti-cancer drug (e.g., a rubicin such as doxorubicin) in the cryopreserved anti-cancer drug-loaded platelets leads to a reduced accumulation in or around the tumor cells as compared to the same dose of the anti-cancer drug in a liposomal formulation. However, in some illustrative embodiments, despite this, the anti-cancer drug-loaded platelets are at least as effective, or more effective at killing cancer cells in the tumor and/or otherwise reducing tumor burden in the subject compared to the same dose of the anti-cancer drug (e.g., a rubicin such as as doxorubicin) in a liposomal formulation. In some embodiments, the accumulation can be at least 1.2, 1.4, 1.5, 1.75, 2, 3, 4, or 5 fold, or between 1.2 to 10, 1.5 to 10, 1.2 to 8, 1.2 to 6, 1.2 to 5, 1.2 to 4, 1.2 to 3, 1.5 to 10, 2 to 10, 3 to 10, or 5 to 10 fold less in an effective dose of the anti-cancer drug in the cryopreserved anti-cancer drug loaded platelets as compared to a liposomal formulation of the anti-cancer drug. In some embodiments, an effective dose of the anti-cancer drug in the cryopreserved anti-cancer drug loaded platelets leads to an increased assimilation in or around the tumor cells as compared to a dose of the anti-cancer drug in a liposomal formulation. In some embodiments, the assimilation can be at least 1.2, 1.4, 1.5, 1.75, 2, 3, 4, or 5 fold, or between 1.2 to 10, 1.5 to 10, 1.2 to 8, 1.2 to 6, 1.2 to 5, 1.2 to 4, 1.2 to 3, 1.5 to 10, 2 to 10, 3 to 10, or 5 to 10 fold more in an effective dose of the anti-cancer drug in the cryopreserved anti-cancer drug loaded platelets as compared to a liposomal formulation of the anti-cancer drug.

In some embodiments of any aspects or embodiments herein that include anti-cancer drug-loaded platelet derivatives, a method for preparing anti-cancer drug-loaded platelet derivatives, a method for administering an anti-cancer agent to a subject, or anti-cancer drug-loaded platelet derivatives used in or obtained by any of the methods herein, an effective dose of anti-cancer drug-loaded platelet derivatives comprises the anti-cancer drug in an amount and/or concentration that is at least 1.2, 1.3, 1.4, or 1.5 fold lower than a regulatory agency-approved dose of the anti-cancer drug that is present in a liposomal formulation. In some embodiments, an effective dose of the anti-cancer drug in the anti-cancer drug-loaded platelet derivatives comprises the anti-cancer drug that is 1.5 to 10, 1.5 to 9, 1.5 to 8, 1.5 to 6, 1.5 to 5, 1.5 to 4, 1.5 to 3, 2 to 10, 3, to 10, 4 to 10, or 5 to 10 fold lower in amount and/or concentration than the dose of the anti-cancer drug that is present in a liposomal formulation of the anti-cancer drug that is administered to the subject. In some embodiments, the anti-cancer drug can be any drug disclosed herein, and in illustrative embodiments, the anti-cancer drug comprises a rubicin, for example doxorubicin, or any salts thereof. In some embodiments, an effective dose of the anti-cancer drug in the anti-cancer drug loaded platelet derivatives more effectively reduces a tumor burden, kills cancer or tumor cells, or reduces size of a tumor in the subject as compared to a dose of the anti-cancer drug that is identical or more than that of the anti-cancer drug in the anti-cancer drug loaded platelet derivatives, but present in a liposomal formulation that is administered to the subject. In some embodiments, the reduction in tumor burden, killing of cancer or tumor cells, or reduction in the size of a tumor can be at least 1.2, 1.4, 1.5, 1.75, 2, 3, 4, or 5 fold effective, or between 1.2 to 10, 1.5 to 10, 1.2 to 8, 1.2 to 6, 1.2 to 5, 1.2 to 4, 1.2 to 3, 1.5 to 10, 2 to 10, 3 to 10, or 5 to 10 fold effective in an effective dose of the anti-cancer drug in the anti-cancer drug loaded platelet derivatives as compared to a liposomal formulation of the anti-cancer drug. In some embodiments, an effective dose of the anti-cancer drug (e.g., a rubicin such as as doxorubicin) in the anti-cancer drug loaded platelet derivatives leads to a reduced accumulation in or around the tumor cells as compared to a dose of the anti-cancer drug in a liposomal formulation. In some embodiments, the accumulation can be at least 1.2, 1.4, 1.5, 1.75, 2, 3, 4, or 5 fold, or between 1.2 to 10, 1.5 to 10, 1.2 to 8, 1.2 to 6, 1.2 to 5, 1.2 to 4, 1.2 to 3, 1.5 to 10, 2 to 10, 3 to 10, or 5 to 10 fold less in an effective dose of the anti-cancer drug in the anti-cancer drug loaded platelet derivatives as compared to a liposomal formulation of the anti-cancer drug. In some embodiments, an effective dose of the anti-cancer drug in the anti-cancer drug loaded platelet derivatives leads to an increased assimilation in or around the tumor cells as compared to a dose of the anti-cancer drug in a liposomal formulation. In some embodiments, the assimilation can be at least 1.2, 1.4, 1.5, 1.75, 2, 3, 4, or 5 fold, or between 1.2 to 10, 1.5 to 10, 1.2 to 8, 1.2 to 6, 1.2 to 5, 1.2 to 4, 1.2 to 3, 1.5 to 10, 2 to 10, 3 to 10, or 5 to 10 fold more in an effective dose of the anti-cancer drug in the anti-cancer drug loaded platelet derivatives as compared to a liposomal formulation of the anti-cancer drug.

In some embodiments of any aspects or embodiments herein that include a method for administering an anti-cancer drug to a subject, the subject has cancer, and the subject is other than a contraindicated person, wherein the contraindicated person has a stent, has a heart condition, had a diagnosed blood clot, and/or has an effective amount of an anti-platelet agent in their bloodstream. In some embodiments, method includes a subject is other than the person having the effective amount of the anti-platelet agent in their bloodstream. In some embodiments, a method include a subject ceased being administered or ceased self-administering the anti-platelet agent such that the anti-platelet agent is no longer present at a detectable level in their blood stream, or is present in their bloodstream at an ineffective amount, at the time of administering the therapeutically effective dose of the anti-cancer drug-loaded platelet derivatives or cryopreserved anti-cancer drug-loaded platelets, in some embodiments, the anti-platelet agent is a GPIIb/IIIa inhibitor. In some embodiments, a subject has cancer, and the subject has one or more of the following indications: has a stent, has a heart condition, had a diagnosed blood clot, and has an effective amount of an anti-platelet agent in their bloodstream. In some embodiments, the anti-cancer drug-loaded platelet derivatives, or the cryopreserved anti-cancer drug-loaded platelets are stored for at least 2 days before the administering. In some embodiments, a method further comprises, after the storing and before the administering, preparing the anti-cancer drug loaded platelet derivatives for administration by rehydrating the anti-cancer drug-loaded platelet derivatives, or preparing the cryopreserved anti-cancer drug-loaded platelets for administration by thawing the cryopreserved anti-cancer drug-loaded platelets, in some embodiments, the anti-cancer drug is doxorubicin. In some embodiments, cancer cells in the subject comprise one or more tumor-associated markers selected from podoplanin, galectin-3, or CD-44. In some embodiments, administering is performed by administering a therapeutically effective dose of the anti-cancer drug-loaded platelet derivatives. In some embodiments, administering is performed by administering a therapeutically effective dose of the cryopreserved anti-cancer drug-loaded platelets. In some embodiments, the cryopreserved anti-cancer drug-loaded platelets comprise 0.5-5.5% DMSO, 0.5-3% glycerol, and 3-10% polysucrose. In some embodiments, administering comprises a) preparing anti-cancer drug loaded platelet derivatives, or cryopreserved anti-cancer drug-loaded platelets by loading the anti-cancer drug into platelets and lyophilizing or freezing the platelets to prepare the anti-cancer drug loaded platelet derivatives, or the cryopreserved anti-cancer drug-loaded platelets, respectively, wherein the anti-cancer drug loaded platelet derivatives, or the cryopreserved anti-cancer drug-loaded platelets comprise the anti-cancer drug; b) storing the anti-cancer drug loaded freeze-dried platelet derivatives, or the cryopreserved anti-cancer drug-loaded platelets for at least 1 day, 2, 3, 4, 5, 7, 8, 9, 10 days, 1 month, 2, 4, 6, 8, 10, or 12 months; and c) administering a therapeutically effective dose of the anti-cancer drug-loaded platelet derivatives or the cryopreserved anti-cancer drug-loaded platelets to the subject. In illustrative embodiments, the anti-cancer drug comprises doxorubicin, or a salt of doxorubicin, to form doxorubicin loaded platelet derivatives or cryopreserved doxorubicin loaded platelets. In some embodiments, an effective dose of the anti-cancer drug in the anti-cancer drug-loaded platelet derivatives or in the cryopreserved anti-cancer drug-loaded platelets comprises the anti-cancer drug that is at least 1.1, 1.2, 1.3, 1.4, or 1.5 fold lower in concentration than a regulatory agency-approved dose of the anti-cancer drug that is present in a liposomal formulation. In some embodiments, an effective dose of the anti-cancer drug in the anti-cancer drug-loaded platelet derivatives or in the cryopreserved anti-cancer drug-loaded platelets comprises the anti-cancer drug that is 1.5 to 10, 1.2 to 10, 1.2 to 8, 1.2 to 6, 1.2 to 5, 1.2 to 4, 1.2 to 3, 1.5 to 10, 2 to 10, 3 to 10, or 5 to 10 fold lower in concentration than the dose of the anti-cancer drug that is present in a liposomal formulation of the anti-cancer drug that is administered to the subject.

In some embodiments of any aspects or embodiments herein that include cryopreserved anti-cancer drug-loaded platelets, a method for preparing cryopreserved anti-cancer drug-loaded platelets, or cryopreserved anti-cancer drug-loaded platelets used in or obtained by any of the methods herein, the cryopreserved anti-cancer drug-loaded platelets are stored at a temperature in the range of −75° C. to −85° C. In some embodiments, the cryopreserved anti-cancer drug-loaded platelets are stored at a temperature in the range of −15° C. to −70° C., −50° C. to −85° C., −55° C. to −85° C., −60° C. to −85° C., or −75° C. to −85° C. In some embodiments, the cryopreserved anti-cancer drug-loaded platelets are stored for between 2, 3, 4, 5, 6, 7, 10, or 14 days on the low end of the range, and 18 days on the high end, between 2 days on the low end of the range, and 7, 8, 10, 14, 18, 28, 30, 45, or 60 days on the high end, between 7 days on the low end of the range, and 8, 10, 14, 18, 28, 30, 45, or 60 days on the high end, or between 10 days on the low end of the range, and 14, 18, 28, 30, 45, or 60 days on the high end. In some embodiments, the cryopreserved anti-cancer drug-loaded platelets are stored for at least 1 month, 2, 3, 4, 6, 8, 10, or 12 months, in illustrative embodiments, before administering to a subject. In some embodiments, the cryopreserved anti-cancer drug-loaded platelets retain at least 30%, 40%, 45%, 50%, 55%, 60%, 65%, 70%, or 75% of the anti-cancer drug after the storing, in illustrative embodiments, the anti-cancer drug is doxorubicin. In some embodiments, the loading buffer comprises a monosaccharide, and a disaccharide, and further comprises polysucrose. In some embodiments, the anti-cancer drug in the cryopreserved anti-cancer drug-loaded platelets is associated with the cryopreserved platelets without the requirement of any linker. In some embodiments, the linker is a peptide or any other molecule having a specificity to any component of platelets, for example, platelet membrane. In some embodiments, the anti-cancer drug in the cryopreserved anti-cancer drug-loaded platelets is associated with the cryopreserved platelets by a linker, wherein the linker is a peptide or any other molecule that does not have a specificity to any components of platelets, for example, platelet membrane. In some embodiments, provided herein is a composition comprising cryopreserved anti-cancer drug-loaded platelets, prepared by any of the methods herein.

In some embodiments of any aspects or embodiments herein that include anti-cancer drug-loaded platelet derivatives, a method for preparing anti-cancer drug-loaded platelet derivatives, or anti-cancer drug-loaded platelet derivatives used in or obtained by any of the methods herein, the anti-cancer drug-loaded platelet derivatives are stored for between 2, 3, 4, 5, 6, 7, 10, or 14 days on the low end of the range, and 18 days on the high end, between 2, 3, 4, 5, 6, 7, 10, or 14 days on the low end of the range, and 28 days on the high end, or between 2, 3, 4, 5, 6, 7, 10, or 14 days on the low end of the range, and 20 days on the high end, between 2 days on the low end of the range, and 7, 8, 10, 14, 18, 28, 30, 45, or 60 days on the high end, between 7 days on the low end of the range, and 8, 10, 14, 18, 28, 30, 45, or 60 days on the high end, or between 10 days on the low end of the range, and 14, 18, 28, 30, 45, or 60 days on the high end. In some embodiments, the anti-cancer drug-loaded platelet derivatives are stored for at least 1 month, 2, 4, 6, 8, 10, 12 months, 1 year, 2, 4, 5, 6, 8, or 10 years, in illustrative embodiments at a temperature in a range of 10° C. to 30° C., 20° C. to 30° C., 20° C. to 28° C., 20° C. to 26° C., 20° C. to 25° C., 20° C. to 23° C., 22° C. to 28° C., or 23° C. to 27° C. In some embodiments, the anti-cancer drug-loaded platelet derivatives are stored at a temperature of 25° C.+/−5° C., 25° C.+/−4° C., 25° C.+/−3° C., 25° C.+/−2° C., or 25° C.+/−1° C. In some embodiments, the anti-cancer drug-loaded platelet derivatives are stored for a time period in the range of 1 month to 10 years, 1 month to 8 years, 1 month to 5 years, or 1 month to 3 years. In some embodiments, the anti-cancer drug-loaded platelet derivatives herein upon storing can retain at least 25%, 30%, 35%, 40%, 45%, 50%, 55%, or 60% of the anti-cancer drug. For example, the anti-cancer drug-loaded platelet derivatives herein upon storing can retain 25% to 95%, 25% to 90%, 25% to 85%, 25% to 80%, 25% to 75%, 25% to 70%, 25% to 65%, 25% to 60%, 25% to 55%, 25% to 50%, 30% to 95%, 40% to 95%, 45% to 95%, 50% to 95%, 55% to 95%, 60% to 95%, or 65% to 95% of the anti-cancer drug upon storing. In some embodiments, the anti-cancer drug is doxorubicin, and the doxorubicin loaded-platelet derivatives retain 4-25, 4-20, 4-15, or 10-25 fg/plt of doxorubicin upon storing. In some embodiments, the concentration of the anti-cancer drug per platelet derivatives or platelets in the anti-cancer drug loaded platelet derivatives varies within 5% to 40%, 5% to 35%, 5% to 30%, 5% to 25%, or 5% to 20% during the storing. In some embodiments, the concentration of the doxorubicin-loaded platelet derivatives is in the range of 1.0×10⁶/μl to 2.5×10⁶/μl, 1.1×10⁶/μl to 1.8×10⁶/μl, 1.3×10⁶/μl to 1.8×10⁶/μl, or 1.3×10⁶/μl to 2×10⁶/μl during the storing. In some embodiments, the loading buffer comprises a monosaccharide, and a disaccharide, and further comprises polysucrose. In some embodiments, the anti-cancer drug in the anti-cancer drug-loaded platelet derivatives is associated with the platelet derivatives without the requirement of any linker. In some embodiments, the linker is a peptide having a specificity to the platelet membrane. In some embodiments, provided herein is a composition comprising anti-cancer drug-loaded platelet derivatives, prepared by any of the methods herein.

In some embodiments of any aspects or embodiments herein that include cryopreserved anti-cancer drug-loaded platelets, a method for preparing cryopreserved anti-cancer drug-loaded platelets, or cryopreserved anti-cancer drug-loaded platelets used in or obtained by any of the methods herein, the cryopreserved anti-cancer drug-loaded platelets are stored at a temperature in the range of −60° C. to −90° C., −65° C. to −90° C., −70° C. to −90° C., −75° C. to −90° C., −75° C. to −85° C., or −78° C. to −82° C. In some embodiments, the cryopreserved anti-cancer drug-loaded platelets are stored at a temperature in the range of −10° C. to −30° C., −15° C. to −50° C., −15° C. to −45° C., −15° C. to −40° C., −15° C. to −35° C., −15° C. to −30° C., −15° C. to −25° C., or −18° C. to −23° C. In some embodiments, the cryopreserved anti-cancer drug-loaded platelets are stored at a temperature of −20° C.+/−5° C., −20° C.+/−4° C., −20° C.+/−3° C., −20° C.+/−2° C., or −20° C.+/−1° C. In some embodiments, In some embodiments, the cryopreserved anti-cancer drug-loaded platelets are stored at a temperature in the range of −10° C. to −30° C. for at least 1 day, 2, 3, 4, 5, 6, 7, 8, 9, 10, 14, 20, 25, 28 days, 1 month, 2, 4, 6, 8, 10 months, 1 year, 2, 4, 6, 8, or 10 years. In some embodiments, the cryopreserved doxorubicin loaded platelets retain at least 25%, 30%, 35%, 40%, 45%, 50%, 55%, 60%, 70%, or 75% of the loaded anti-cancer drug upon storing. In some embodiments, the cryopreserved doxorubicin loaded platelets retain 25% to 99%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 30% to 95%, 40% to 95%, 45% to 95%, 50% to 95%, 55% to 95%, 60% to 95%, 65% to 95%, or 50% to 99% of the loaded anti-cancer drug upon storing. In some embodiments, the anti-cancer drug is doxorubicin, and the cryopreserved doxorubicin loaded platelets retain 4-25, 4-20, 4-15, or 10-25 fg/plt of doxorubicin upon storing. In some embodiments, the concentration of the anti-cancer drug in the cryopreserved anti-cancer drug loaded platelets varies within 5% to 40%, 5% to 35%, 5% to 30%, 5% to 25%, or 5% to 20% during the storing. In some embodiments, the cryopreserved anti-cancer drug loaded platelets retain at least 30%, 35%, 40%, 45%, 50%, 55%, 60%, 65%, or 70% of the anti-cancer drug after thawing as compared to before the cryopreservation. In some embodiments, the concentration of the anti-cancer drug loaded platelet derivatives, the doxorubicin-loaded platelet derivatives, the cryopreserved anti-cancer drug loaded platelets, or the cryopreserved doxorubicin-loaded platelets is in the range of 1.0×10⁶/μl to 2.5×10⁶/μl, 1.1×10⁶/μl to 1.8×10⁶/μl, 1.3×10⁶/μl to 1.8×10⁶/μl, or 1.3×10⁶/μl to 2×10⁶/μl during the storing.

In some embodiments of any of aspects or embodiments herein that include a method for testing platelet derivatives or cryopreserved platelets for the ability to deliver an anti-cancer drug to a cancer cell, the marker is one or more of CD41, CD61, and CD62. In some embodiments, the method comprises testing the platelet derivatives, and in illustrative embodiments, the platelet derivatives are anti-cancer drug-loaded platelet derivatives. In some embodiments, anti-cancer drug-loaded platelet derivatives are doxorubicin-loaded platelet derivatives. In some embodiments, the presence of the interaction indicates passing a quality control test by the platelet derivatives, the anti-cancer drug-loaded platelet derivatives, or the doxorubicin-loaded platelet derivatives of a specific lot. In some embodiments, the method comprises testing the cryopreserved platelets, in illustrative embodiments, the cryopreserved platelets are cryopreserved anti-cancer drug loaded platelets. In some embodiments, the cryopreserved anti-cancer drug loaded platelets are cryopreserved doxorubicin-loaded platelets. In some embodiments, the tumor-associated marker comprises one or more of podoplanin, galectin-3, and CD-44. In some embodiments, the presence of the interaction indicates passing a quality control test by the cryopreserved platelets, the cryopreserved anti-cancer drug-loaded platelets, or the cryopreserved doxorubicin-loaded platelets of a specific lot.

In some embodiments of any of aspects or embodiments herein that include an anti-cancer drug, cryopreserved anti-cancer drug-loaded platelets, anti-cancer drug loaded platelet derivatives, or any of the methods that uses or produces cryopreserved anti-cancer drug-loaded platelets or anti-cancer drug loaded platelet derivatives herein, or that includes a method of administrating an anti-cancer drug, the anti-cancer drug is selected from the group consisting of vemurafenib, dabrafenib, encorafenib, BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, LXH254, trametinib (GSK1120212), cobimetinib, binimetinib (MEK162), selumetinib (AZD6244), PD0325901, MSC1936369B, SHR7390, TAK-733, CS3006, WX-554, PD98059, CI1040 (PD184352), hypothemycin, FRI-20 (ON-01060), VTX-Ile, 25-OH-D3-3-BE (B3CD, bromoacetoxycalcidiol), FR-180204, AEZ-131 (AEZS—131), AEZS—136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-oxozeaenol, 5-iodotubercidin, GDC0994, ONC201, buparlisib (BKM120), alpelisib (BYL719), WX-037, copanlisib (BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS—5584 (SB2343), PKI-402, wortmannin, LY294002, PI-103, rigosertib, XL-765, LY2023414, SAR260301, KIN-193 (AZD-6428), GS-9820, AMG319, GSK2636771, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, erucylphophocholine, erufosine, SR13668, OSU-A9, PH-316, PHT-427, PIT-1, DM-PIT-1, triciribine (triciribine phosphate monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, MLN0128, AZD-2014, CC-223, AZD2014, CC-115, everolimus (RAD001), temsirolimus (CCI-779), ridaforolimus (AP-23573), tipifarnib, BMS-214662, L778123, L744832, FTI-277, PRI-724, CWP232291, PNU74654, PKF115-584, PKF118-744, PKF118-310, PFK222-815, CGP 049090, ZTM000990, BC21, methyl 3-{[(4-methylphenyl) sulfonyl]amino}benzoate (MSAB), AV65, iCRT3, iCRT5, iCRT14, SM04554, LGK 974, XAV939, curcumin, DIF-1, genistein, NSC668036, FJ9, BML-286 (3289-8625), IWP, IWP-1, IWP-2, JW55, G007-LK, pyrvinium, foxy-5, Wnt-5a, ipafricept (OMP-54F28), SM04690, SM04755, nutlin-3a, IWR1, JW74, okadaic acid, SB239063, SB203580, adenosine diphosphate (hydroxymethyl) pyrrolidinediol (ADP-HPD), 2-[4-(4-fluorophenyl) piperazin-1-yl]-6-methylpyrimidin-4 (3H)-one, PJ34, J01-017a, IC261, PF670462, bosutinib, PHA665752, imatinib, ICG-001, Rp-8-Br-CAMP, SDX-308, WNT974, CGX1321, ETC-1922159, AD-REIC/Dkk3, WIKI4, windorphen, NTRC 0066-0, CFI-402257, a (5,6-dihydro)pyrimido[4,5-e]indolizine, BOS172722, S63845, AZD5991, AMG 176, 483-LM, MIK665, TASIN-1 (Truncated APC Selective Inhibitor), osimertinib (AZD9291, merelectinib, TAGRISSO™), erlotinib, gefitinib, neratinib (HKI-272), lapatinib, vetanib, rociletinib (CO-1686), olmutinib (HM61713, BI-1482694), naquotinib (ASP8273), nazartinib (EGF816, NVS—816), PF-06747775, icotinib (BPI-2009H), afatinib (BIBW 2992), dacomitinib (PF-00299804, PF-804, PF-299, PF-299804), avitinib (AC0010), AC0010MA EAI045, canertinib (CI-1033), poziotinib (NOV120101, HM781-36B), AV-412, WZ4002, brigatinib (AP26113), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS—10296, GW572016, pyrotinib (SHR1258), palbociclib, ribociclib, abemaciclib, olaparib, veliparib, iniparib, rucaparib, CEP-9722, E7016, E7449, PRN1371, BLU9931, FIIN-4, H3B-6527, NVP-BGJ398, ARQ087, TAS—120, CH5183284, Debio 1347, INCB054828, JNJ-42756493 (erdafitinib), rogaratinib (BAY1163877), FIIN-2, LY2874455, lenvatinib (E7080), ponatinib (AP24534), regorafenib (BAY 73-4506), dovitinib (TKI258), lucitanib (E3810), cediranib (AZD2171), nintedanib (BIBF 1120), brivanib (BMS-540215), ASP5878, AZD4547, BGJ398 (infigratinib), E7090, HMPL-453, MAX-40279, XL999, orantinib (SU6668), pazopanib, anlotinib, AL3818, PRIMA-1 (p53 reactivation induction of massive apoptosis-1), APR-246 (PRIMA-1MET), PK11007, PK7088, zinc metallochaperone-1 (ZMC1; NSC319726/ZMC 1), COTI-2, CP-31398, STIMA-1 (SH Group-Targeting Compound That Induces Massive Apoptosis), MIRA-1 (NSC19630), MIRA-2, MIRA-3, RITA (NSC652287), chetomin (CTM), stictic acid (NSC87511), p53R3, SCH529074, WR-1065, gambogic acid, spautin-1, YK-3-237, NSC59984, disulfiram (DSF), G418, RETRA (reactivate transcriptional activity), PD0166285, 17-AAG, geldanamycin, ganetespib, AUY922, IPI-504, vorinostat/SAHA, romidepsin/depsipeptide, HBI-8000, RG7112 (RO5045337), RO5503781, MI-773 (SAR405838), DS—3032b, AM-8553, AMG 232, MI-219, MI-713, MI-888, TDP521252, NSC279287, PXN822, ATSP-7041, spiroligomer, PK083, PK5174, PK5196, nutlin 3a, RG7388, Ro-2443, FTY-720, ceramide, OP449, vatalanib (PTK787/ZK222584), TKI-538, sunitinib (SU11248), thalidomide, lenalidomide, axitinib (AG013736), RXC0004, ETC-159, LGK974, WNT-C59, AZD8931, AST1306, CP724714, CUDC101, TAK285, AC480, DXL-702, E-75, PX-104.1, ZW25, CP-724714, irbinitinib (ARRY-380, ONT-380), TAS0728, AST-1306, AEE-788, perlitinib (EKB-569), PKI-166, D-69491, HKI-357, AC-480 (BMS-599626), RB-200 h, ARRY-334543 (ARRY-543, ASLAN001), CUDC-101, IDM-1, decitabine, cytosine arabinoside, ORY1001 (RG6016), GSK2879552, INCB059872, IMG7289, CC90011, MI1, MI2, MI3, Mi2-2 (MI-2-2), MI463, MI503, MIV-6R, EPZ004777, EPZ-5676, SGC0946, CN-SAH, SYC-522, SAH, SYC-534, MM-101, MM-102, MM-103, MM-401, WDR5-0101, WDR5-0102, WDR5-0103, OICR-9429, tivantinib (ARQ 197), golvatinib (E7050), cabozantinib (XL 184, BMS-907351), foretinib (GSK1363089), crizotinib (PF-02341066), MK-2461, BPI-9016M, TQ-B3139, MGCD265, MK-8033, capmatinib (INC280, INCB28060), tepotinib (MSC2156119J, EMD1214063), CE-35562, AMG-337, AMG-458, PHA-665725, PF-04217903, SU11274, PHA-665752, HS—10241, ARGX-111, glumetinib (SCC244), EMD 1204831, AZD6094 (savolitinib, volitinib, HMPL-504), PLB1001, ABT-700, AMG 208, INCB028060, AL2846, HTI-1066, PT2385, PT2977, 17 allylamino-17-demethoxygeldanamycin, eribulin (E389, ER-086526), ibrutinib (PCI-32675) (1-[(3R)-3-[4-amino-3-(4-phenoxyphenyl) pyrazolo[3,4-d]pyrimidin-1-yl]piperidin-1-yl]prop-2-en-1-one), AC0058 (AC0058TA), N-(3-((2-((3-fluoro-4-(4-methylpiperazin-1-yl)phenyl)amino)-7H-pyrrolo[2,3-d]pyrimidin-4-yl)oxy)phenyl) acrylamide, acalabrutinib (ACP-196, rINN) (4-[8-amino-3-[(2S)-1-but-2-ynoylpyrrolidin-2-yl]imidazo[1,5-alpyrazin-1-yl]-N-pyridin-2-ylbenzamide), zanubrutinib (BGB-3111) ((7R)-2-(4-phenoxyphenyl)-7-(1-prop-2-enoylpiperidin-4-yl)-1,5,6,7-tetrahydropyrazolo[1,5-a]pyrimidine-3-carboxamide), spebrutinib (AVL-292, 1202757-89-8, Cc-292) (N-[3-[5-fluoro-2-[4-(2-methoxyethoxy)anilino]pyrimidin-4-yl]amino]phenyl]prop-2-enamide), poseltinib (HM71224, LY3337641) (N-[3-[2-[4-(4-methylpiperazin-1-yl)anilino]furo[3,2-d]pyrimidin-4-yl]oxyphenyl]prop-2-enamide), evobrutinib (MSC 2364447, M-2951) (1-[4-[6-amino-5-(4-phenoxyphenyl)pyrimidin-4-yl]amino]methyl]piperidin-1-yl]prop-2-en-1-one), tirabrutinib (ONO-4059, GS-4059, ONO/GS-4059, ONO-WG-307) (1-[4-[6-amino-5-(4-phenoxyphenyl)pyrimidin-4-yl]amino]methyl]piperidin-1-yl]prop-2-en-1-one), vecabrutinib (SNS-062) ((3R,4S)-1-(6-amino-5-fluoropyrimidin-4-yl)-3-[(3R)-3-[3-chloro-5-(trifluoromethyl) anilino]-2-oxopiperidin-1-yl]piperidine-4-carboxamide), dasatinib (BMS-354825) (N-(2-chloro-6-methylphenyl)-2-[6-[4-(2-hydroxyethyl) piperazin-1-yl]-2-methylpyrimidin-4-yl]amino]-1,3-thiazole-5-carboxamide), PRN1008, PRN473, ABBV-105, CG-806, ARQ 531, BIIB068, AS871, CB1763, CB988, GDC-0853, RN486, GNE-504, GNE-309, BTK Max, CT-1530, CGI-1746, CGI-560, LFM A13, TP-0158, DTRMWXHS-12, CNX-774, entrectinib, nilotinib, 1-((3S,4R)-4-(3-fluorophenyl)-1-(2-methoxyethyl) pyrrolidin-3-yl)-3-(4-methyl-3-(2-methylpyrimidin-5-yl)-1-phenyl-1H-pyrazol-5-yl) urea, AG 879, AR-772, AR-786, AR-256, AR-618, AZ-23, AZ623, DS—6051, Go 6976, GNF-5837, GTx-186, GW 441756, LOXO-101, LOXO-195, MGCD516, PLX7486, RXDX101, TPX-0005, TSR-011, venetoclax (ABT-199, RG7601, GDC-0199), navitoclax (ABT-263), ABT-737, TW-37, sabutoclax, obatoclax, BIX-01294 (BIX), UNC0638, A-366, UNC0642, DCG066, UNC0321, BRD 4770, UNC 0224, UNC 0646, UNC0631, BIX-01338, INNO-406, KX2-391, saracatinib, PP1, PP2, ruxolitinib, lestaurtinib (CEP-701), momelotinib (GS-0387, CYT-387), pacritinib (SB1518), fedratinib (SAR302503), BI2536, BI6727, GSK461364, amsacrine, azacitidine, busulfan, carboplatin, capecitabine, chlorambucil, cisplatin, cyclophosphamide, cytarabine, dacarbazine, daunorubicin, docetaxel, doxifluridine, doxorubicin, epirubicin, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, streptozocin, tafluposide, temozolomide, teniposide, tioguanine, topotecan, uramustine, valrubicin, vinblastine, vincristine, vindesine, and vinorelbine. In some embodiments, the anti-cancer drug is a chemotherapy drug. In some embodiments, the chemotherapy drug is selected from an alkylating agent, an antimetabolite, an anthracycline, an antitumor antibiotic, a platinum-containing compound, or a plant alkaloid. In some embodiments, the chemotherapy drug is methotrexate, pralatrexate, doxorubicin, vincristine, mitoxantrone, etoposide, or bleomycin. In some embodiments, the chemotherapy drug is an anthracycline or doxorubicin. In some embodiments, the anti-cancer drug is selected from a small molecule, and in some embodiments, the small molecule is selected from a kinase inhibitor, a receptor tyrosine kinase inhibitor, a non-receptor tyrosine kinase inhibitor, a serine/threonine kinase inhibitor, an epigenetic inhibitor, a BCL-2 inhibitor, a Hedgehog pathway inhibitor, a proteasome inhibitor, or a PARP inhibitor. In some embodiments, the anti-cancer drug is a biologic. In some embodiments, the anti-cancer drug is one or more antibody-drug conjugate (ADC) payloads, in some cases, the ADC payload is selected from a tubulin inhibitor payload or a DNA-damaging agent, typically, the tubulin inhibitor payloads are selected from an auristatin, or a maytansine. In some embodiments, the DNA-damaging agent is selected from a calicheamicin, a Duocarymycin, a Pyrrolobenzodiazepine (PBD) dimer, or an amatoxin. In some embodiments, the ADC payload is an antibiotic of the ADC. In some embodiments, the ADC payload is an enzyme of the ADC. In some embodiments, the payload is a protein toxin or immunotoxin of the ADC. In some embodiments, the anti-cancer drug is a therapeutic agent that is approved for treating at least one type of cancer by a regulatory agency, in some embodiments, the regulatory agency is tasked with testing the safety and efficacy of a therapeutic agent. In some embodiments, the regulatory agency is one of U.S. Food and Drug Administration (FDA), European Medicines Agency (EPA), and China Food and Drug Administration. In some embodiments, the anti-cancer drug is an active agent of the therapeutic agent.

The following non-limiting examples are provided purely by way of illustration of exemplary embodiments, and in no way limit the scope and spirit of the present disclosure.

### EXAMPLES

### Example 1: Preparation of Doxorubicin Loaded Cryopreserved Platelets (DOX-CPP) and Doxorubicin Loaded Freeze-Dried Platelet-Derived Hemostat (DOX-FPH)

Doxorubicin (DOX) was loaded onto apheresis platelets to create DOX-CPP and DOX-FPH. Apheresis platelet units (Atlanta Blood Services) were acidified with acid citrate dextrose (ACD) to establish a pH of 6.6-6.8. Cell count was obtained using the AcT Diff Hematology Analyzer (Beckman Coulter). The APU was then centrifuged at 1470×g for 20 minutes at 21° C. After centrifugation, the platelet poor plasma (PPP) was removed and the platelet pellet was resuspended at 2500×10³ cells/μL in 10 ml of loading buffer and 10 ml of PBS with 2 μM of each platelet aggregation inhibitors PGE1, GR144053, and EGTA (Tocris, catalog no. 1620, 1263, and 2807 respectively) and protease inhibitor (thermos Scientific, catalog no. PIA32953) a. A non-limiting example of the loading buffer composition is detailed below.

TABLE 5
Loading Buffer

| Loading Buffer | Value | |
|---|---|---|
| Component | (±1%) | |
| HEPES | 7.6 | mM |
| NaCl | 60 | mM |
| KCl | 3.84 | mM |
| Dextrose | 2.4 | mM |
| NaHCO₃ | 9.6 | mM |
| Trehalose | 80 | mM |

| | |
|---|---|
| Ethanol | 0.8% |
| pH | 6.6-6.8 |

Platelets were incubated with inhibitors for 10 minutes at 37° C. to prevent platelet activation. 7 mg of doxorubicin (Sigma Aldrich, catalog no. D1515) was dissolved in 700 μl cell culture grade water. An equal volume of loading buffer or PBS with and without DOX was added for loading or negative control respectively. 2.5 mg of DOX was added into 10 ml of platelets. These sublots were incubated at 37° C. on the rocker for 3 hours in the dark to allow for DOX loading. During the 3-hour incubation 1 μM of PGE1 and GR144053 were resupplied at every hour to inhibit aggregation. Loaded and unloaded platelets were each diluted 12-fold in loading buffer and then centrifugated at 1470×g for 20 minutes at 21° C. to remove any free DOX in the loaded sublot. Free DOX was removed with the supernatant and the remaining loaded or unloaded platelets were resuspended at 2500×10³ in the loading buffer (Table: Loading Buffer) plus 6% polysucrose to create DOX-loaded FPH or loading buffer plus 6% polysucrose containing 1% DMSO and 1% Glycerol to create DOX-loaded CPP.

For the DOX-loaded CPP, a concentration test using AcT Diff was performed for a target of 2000×10³ platelets/μl. 5 ml of the product was aliquoted into 32 ml FEP bag. The bags were transferred to a −80° C. freezer for cryopreservation. As per one of the working lots, a concentration of 1.7×10⁶/μl DOX-loaded CPP has 44.1 μM of doxorubicin with 14.1 fg of doxorubicin/cryopreserved platelet.

For DOX-loaded FPH, a concentration test using AcT Diff was performed for a target of 10,000×10³ platelets/μl. 1 ml of the product was added to prelabeled 5 ml amber vials. A stopper was added, and vial was transferred to the Stellar Lyophilizer with a pre-chilled shelf of −40° C. The product was lyophilized according to the following recipe. After the lyophilization vials were stopped and capped to be transferred to a 80° C. oven for baking for 24 hours. After the baking step, the lyophilized vials were stored at room temperature (approximately between 20° C. to 25° C.) till further use. As per one of the working lots, a concentration of 1.6×10⁶/μl DOX-loaded FPH has 30.3 UM of doxorubicin with 10.3 fg of doxorubicin/platelet derivative. It is understood that a skilled artisan can modify the concentration of DOX-loaded CPP or DOX-loaded FPH to achieve a certain concentration of doxorubicin. Also, it can be understood that the concentration of doxorubicin within the cryopreserved platelets and platelet derivatives can vary depending upon the initial concentration of doxorubicin that is used and also due to variability in performing the method multiple number of times.

TABLE 6
Lyophilization Protocol

| | Temperature | | Time | | Vacuum pressure | |
|---|---|---|---|---|---|---|
| Freezing | | | | | | |
| Ramp to | −40° | C. | 0 | mins | 0 | mTorr |
| Hold at | −40° | C. | 180 | mins | 0 | mTorr |
| Final Freezing | | | | | | |
| Hold at | −40° | C. | 0 | mins | 100 | mTorr |
| Primary Drying | | | | | | |
| Ramp to | −10° | C. | 360 | mins | 0 | mTorr |
| Hold at | −10° | C. | 360 | mins | 0 | mTorr |
| Ramp to | +5° | C. | 180 | mins | 0 | mTorr |
| Hold at | +5° | C. | 360 | mins | 0 | mTorr |
| Ramp to | +30° | C. | 300 | mins | 0 | mTorr |
| Hold at | +30° | C. | 720 | mins | 0 | mTorr |
| Hold at | +30° | C. | 720 | mins | 200 | mTorr |
| Hold at | +30° | C. | 60 | mins | 0 | mTorr |
| Secondary Drying | | | | | | |
| Hold at | +30° | C. | 9999 | mins | 0 | mTorr |
| Total Recipe Time | | | | | | |
| is ~54 hours | | | | | | |

### Example 2: Quantification of DOX Loading in FPH and CPP

Doxorubicin loading was quantified before and after the product was lyophilized to obtain DOX-FPH or cryopreserved to obtain DOX-CPP as per the process of Example 1. Prior to quantifying the loaded DOX, the best optical density (OD) for DOX from a plate reader and standard curve of the DOX was determined.

For determining the best optical density, 1 mg of DOX was dissolved into 100 μl of cell culture water and sonicated for 10 minutes to create the stock solution. The stock solution was then diluted into 100 μg/ml. To get the highest absorbance, plate reader was used to absorbance scan and the highest OD reading was the best wavelength.

For determining the standard curve, the stock solution for the OD was diluted into 625 μg/ml to make a serial dilution with a dilution factor of 0.5. Clear 96 well plate was used to load 100 μl of the samples. The absorbance was read at 490 nm, and a standard curve was created.

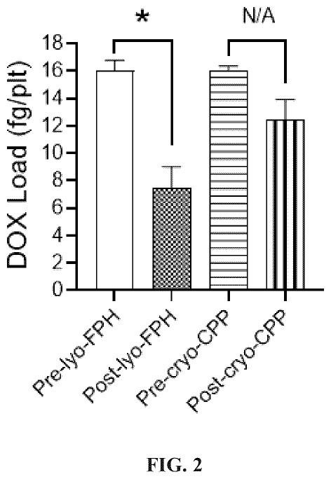

The doxorubicin content was quantified for FPH and CPP pre/post lyophilization and pre/post cryopreservation, respectively. AcT Diff Hematology Analyzer was used to count the cells after loading which included DOX-PBS, DOX-FPH, unloaded FPH, DOX-CPP, and unloaded CPP. 2 ml of DOX loaded CPP/FPH and unloaded CPP/FPH were prepared for a total platelet count of 3.2×10⁹ platelets each. Post cryopreservation, DOX-CPP was removed from the freezer and thawed in a 37° C. water bath for 8 minutes and then rehydrated by adding 25 ml of 0.9% saline. Post lyophilized DOX-FPH was rehydrated with sterile water equivalent to the fill volume prior to lyophilization. All samples were centrifugated at 1470×g for 20 minutes at room temperature. The supernatant was discarded and 200 μl RIPA lysis buffer (25 mM Tris HCl, 150 mM NaCl. 1.0% (v/v) NP-40, 1% Sodium Deoxycholate, 0.1% (w/v) SDS at a pH of 7.6; ThermoFisher Scientific, catalog no 89901) was added to each pellet for 30 minutes. The samples were centrifugated again at 1200×g for 20 minutes at room temperature. The supernatant was collected and 100 μl of the supernatant sample was added to a clear 96 well plate. The absorbance was read at 490 nm and OD reading was compared with the standard curve. FIG. 2 shows the quantification of loaded doxorubicin in pre-lyophilized and post-lyophilized DOX-FPH and pre-cryopreserved and post-cryopreserved DOX-CPP with one of the lots produced by the process of Example 1. Referring to FIG. 2, it can be observed that there is more doxorubicin retained in the cryopreserved platelets in comparison to the freeze-dried platelets. Therefore, given the same number of platelets, cryopreserved DOX-loaded platelets (DOX-CPP) retains more doxorubicin as compared to the lyophilized product (DOX-FPH). Referring to FIG. 2, it can be observed that at least 60% of doxorubicin is retained in DOX-CPP (Post-cryo-CPP) as compared to before cryopreservation (Pre-cryo-CPP). Similarly, it can be observed that at least 40% of doxorubicin is retained in DOX-FPH (Post-lyo-FPH) as compared to before lyophilization (Pre-lyo-FPH).

### Example 3: Stability Study on Cell Counts and Doxorubicin Retention Over Time

The stability of DOX-CPP and DOX-FPH was determined at 0, 2, 3, 5, 9, 14, and 18 days. To obtain stability analysis, the DOX-CPP and the DOX-FPH were prepared as per Example 1, and the highest OD and standard curve was determined as described in Example 2.

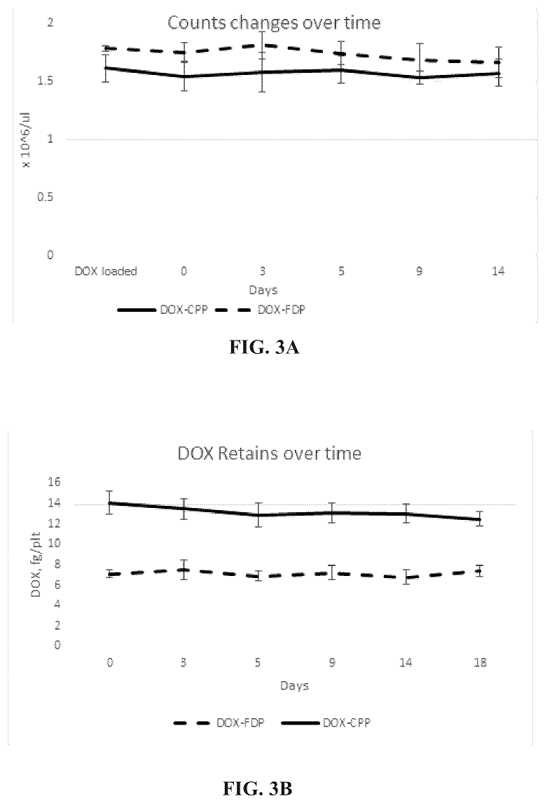

AcT Diff Hematology Analyzer was used to count the cells in all samples which included DOX-PBS, DOX-FPH, unloaded FPH, DOX-CPP, and unloaded CPP. 2 ml of DOX loaded CPP/FPH and unloaded CPP/FPH were prepared for a total platelet count of 3.2×10⁹ platelets each. DOX-CPP and un-loaded CPP were removed from the freezer and thawed in a 37° C. water bath for 8 minutes and then rehydrated by adding 25 ml of 0.9% saline. DOX-FPH and unloaded FPH were rehydrated with sterile water equivalent to the fill volume prior to lyophilization. Both loaded and unloaded CPP and FPH were looked at to determine whether DOX had changed the cell counts. All samples were centrifugated at 1470×g for 20 minutes at room temperature. The supernatant was discarded and 200 μl RIPA lysis buffer (25 mM Tris HCl, 150 mM NaCl, 1.0% (v/v) NP-40, 1% Sodium Deoxycholate, 0.1% (w/v) SDS at a pH of 7.6; ThermoFisher Scientific, catalog no 89901) was added to each pellet for 30 minutes. The samples were centrifugated again at 1200×g for 20 minutes at room temperature. The supernatant was collected and 100 μl of the supernatant sample was added to a clear 96 well plate. The absorbance was read at 490 nm and OD reading was compared with the standard curve. FIG. 3A and FIG. 3B show the platelet count and doxorubicin retention over time, respectively. It can be observed from FIG. 3A that the platelet count in the case of both DOX-CPP and DOX-FPH varies within the range of 5% to 30% for up to 14 days of storing. From FIG. 3B, it can be observed that DOX-CPP and DOX-FPH retain 70-100% of drug loaded for up to 18 days following the preservation mechanism (cryopreserved or freeze-dried).

### Example 4: Clot Formation Analysis of DOX-CPP and DOX-FPH Using T-TAS®

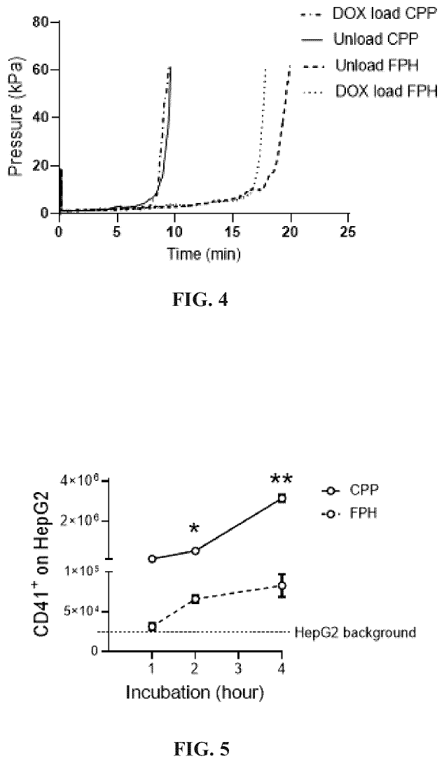

The clot formation of the DOX-CPP and DOX-FPH was compared with unloaded CPP and unloaded FPH to determine if the loaded DOX changes the clot function of the product. DOX-CPP and the DOX-FPH were prepared as per Example 1. DOX-CPP and un-loaded CPP were removed from the freezer and thawed in a 37° C. water bath for 8 minutes and then rehydrated by adding 25 ml of 0.9% saline. DOX-FPH and unloaded FPH were rehydrated with sterile water equivalent to the fill volume prior to lyophilization. AcT Diff Hematology Analyzer was used to count the cells in all samples which included DOX-loading buffer, DOX-FPH, unloaded FPH, DOX-CPP, and unloaded CPP. All samples were prepared in OCTAPLAS® (Octapharma) to create 80 k/μl in octaplas. All samples were created immediately prior to running on the T-TAS. An AR chip was used. Samples were incubated for 5 minutes with periodic inversion of the microcentrifuge tube to mix the sample before running it. After 5 minutes of incubation, 480 μl of each sample was combined with 20 μl of CaCTI reagent and 450 μl of that mixture was immediately pipetted into the reagent reservoir and run on the T-TAS. FIG. 4 shows the clot formation time for DOX-CPP, unloaded CPP, DOX-FPH, and unloaded FPH. The results clearly show that the loading of doxorubicin does not affect the product's ability to clot when compared to the unloaded product. In fact, the DOX-CPP and unloaded CPP have similar clot formation time.

### Example 5: Characterize Cell Interaction Between CPP and FPH to HepG2 Cancer Cells with CD41 Marker Using Flow Cytometry

The receptor mediated surface binding between HepG2 cancer cells and cryopreserved platelets (CPP)/freeze-dried platelet-derived hemostats (FPH) without DOX loading was investigated. CPP and FPH without DOX loading were prepared as per Example 1 but without including or loading with doxorubicin. HepG2 is a cell line exhibiting epithelial-like morphology that was isolated from a hepatocellular carcinoma of a 15-year-old, white male youth with liver cancer. Untreated HepG2 cells served as negative control groups. HepG2 cells were seeded at a density of 10⁵ cells in a 12-well plate (Nunc™ Cell-Culture Treated Multidishes, catalog no: 150628) After incubation for 24 hours, the original medium was replaced with fresh 0.5% FBS DMEM (ATCC-formulated Eagle's Minimum Essential Medium, catalog No. 30-2003) supplemented with CPP or FPH product at an equivalent platelet count of 0.3×10⁶ platelets/μl. The cells were then washed three times with PBS to remove an unbound CPP or FPH. The cells were again washed with cold PBS three times, harvested by a scraper, and then suspended in PBS. Next the cells were incubated with CD41-FTIC platelet marker for 20 minutes followed by washing with PBS. The fluorescence intensity was determined by flow cytometry with the FITC channel. Approximately 10,0000 events for each sample were measured to assess the surface binding between the HepG2 cells and CPP or FPH over three different time periods (1, 2 and 4 hours). FIG. 5 shows the surface interaction between the HepG2 cells and CPP or FPH. Further the experiment confirmed increased surface interaction as a function of time; the longer the HepG2 cell and CPP/FPH were incubated together the higher is their interaction.

### Example 6: Characterize Intracellular Uptake of DOX Loaded Products in HepG2 Cells

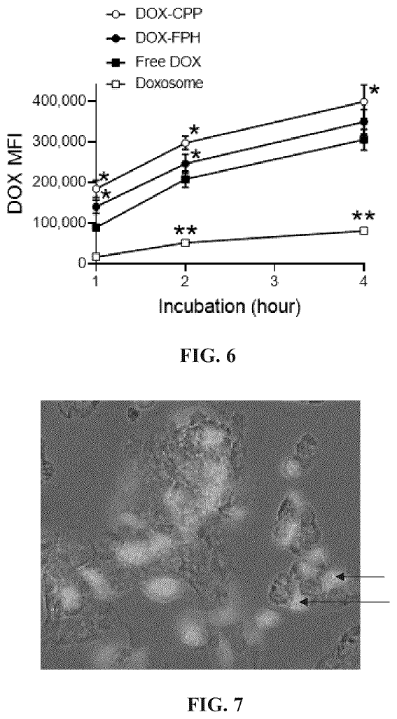

Intracellular uptake of different DOX products into HepG2 cells was investigated. The different DOX products included: DOX-CPP, DOX-FPH, Doxosome® (Encapsulated Nanosciences), and free doxorubicin (Free-DOX). Untreated HepG2 cells served as negative control groups. HepG2 cells were seeded at a density of 10⁵ cells in a 12-well plate (Nunc™ Cell-Culture Treated Multidishes, catalog no: 150628) After incubation for 24 hours, the original medium was replaced with fresh 0.5% FBS DMEM (ATCC-formulated Eagle's Minimum Essential Medium, catalog No. 30-2003) supplemented with DOX-CPP, DOX-FPH, Doxosome, or Free-DOX at an equivalent concentration of 0.6 μM doxorubicin. The approximate concentrations of DOX-CPP, DOX-FPH, and Doxosome to achieve the concentration of 0.6 μM were 0.02×10⁶ DOX-CPP/μl, 0.04×10⁶ DOX-FPH/μl, and 0.3×10⁶ particles/μl, respectively. The cells were then washed three times with PBS to remove an unbound DOX product. The cells were again washed with cold PBS three times, harvested by a scraper, and then suspended in PBS. Next the cells were incubated with CD41-FTIC platelet marker for 20 minutes followed by washing with PBS. The intercellular fluorescence intensity was determined by flow cytometry with the PE-Cy5 channel to measure DOX. Approximately 10,0000 events for each sample were measured to assess DOX product (DOX-CPP, DOX-FPH, Doxosome, Free-DOX) uptake by HepG2 cells. FIG. 6 shows that the intracellular uptake of DOX-CPP and DOX-FPH are greater than either free-DOX or Doxosome, and Doxosome showing the lowest uptake. Also, amongst DOX-CPP and DOX-FPH, DOX-CPP shows higher intracellular uptake.

### Example 7: Intercellular Localization Imaging of DOX-CPP Inside HepG2 Cells

Microscopic image of intracellular localization of DOX-CPP in HepG2 cells was investigated. DOX-CPP was prepared as per the process of Example 1. HepG2 cells were seeded at a density of 10⁵ cells in a 12-well plate (Nunc™ Cell-Culture Treated Multidishes, catalog no: 150628) After incubation for 24 hours, the original medium was replaced with fresh 0.5% FBS DMEM (ATCC-formulated Eagle's Minimum Essential Medium, catalog No. 30-2003) supplemented with DOX-CPP at an equivalent platelet count of 0.3×10⁶ platelets/μl. The cells were then washed three times with cold PBS and fixed with 4% paraformaldehyde for 15 minutes. Fluorescence images were taken using a fluorescence microscope (Olympus). From these images, it can be observed that DOX has been released from the CPP product inside many of the HepG2 cell (See e.g., two representative locations of Dox within HepG2 cells pointed by the black arrows in FIG. 7).

### Example 8: Receptor Mediated Binding Between CPP or FPH with HepG2 Cells

Receptor mediated binding between CPP or FPH with HepG2 cells was investigated by inhibiting either a platelet receptor or a HepG2 receptor. Pooled apheresis platelets were incubated with 50 μM CFDA-Se for 60 minutes at room temperature then acidified to pH of 6.6-6.8 with citric acid. CPP and FPH were then created as disclosed in Example 1 but without doxorubicin. Untreated HepG2 cells served as negative control groups. HepG2 cells were seeded at a density of 10⁵ cells in a 12-well plate (Nunc™ Cell-Culture Treated Multidishes, catalog no: 150628) After incubation for 24 hours, the original medium was replaced at 2 hours with fresh 0.5% FBS DMEM (ATCC-formulated Eagle's Minimum Essential Medium, catalog No. 30-2003).

To determine the effect of platelet receptor inhibition on the binding between CPP/FPH and HepG2 cells first both CPP and FPH were incubated with tirofiban to block the GpIIb/IIIa receptor. Then the HepG2 cells were supplemented with tirofiban treated CPP, tirofiban treated FPH, untreated CPP, or untreated FPH at an equivalent platelet count of 0.3×10⁶ platelets/μl. The cells were then washed with cold PBS three times, harvested by a scraper, and then suspended in PBS. The fluorescence intensity was determined by flow cytometry with the FITC channel. Approximately 10,0000 events for each sample was measured to assess the surface binding between the HepG2 cells and tirofiban treated CPP or FPH.

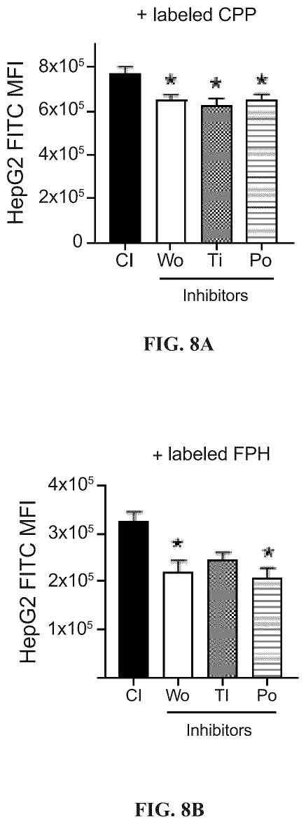

To determine the effect of HepG2 receptor inhibition on the binding between CPP/FPH and HepG2 cells, first HepG2 cells were treated with either anti-podoplanin blocking monoclonal antibody or phagocytosis (wortmannin) inhibitor for 30 minutes. Then the HepG2 cells were incubated with either CPP or FPH. The HepG2 cells were washed with cold PBS three times, harvested by a scraper, and then suspended in PBS. The fluorescence intensity was determined by flow cytometry with the FITC channel. Approximately 10,0000 events for each sample was measured to assess the surface binding between the HepG2 cells CPP or FPH. FIG. 8A and FIG. 8B show the effect of binding between CPP and FPH with HepG2 cells when either one of the inhibitors is introduced. CPP and FPH binding is reduced (16%-30%) in the presence of either one of the inhibitors. The experiment confirms platelet interaction with liver cancer cells includes receptor mediated surface binding and platelet encapsulation via phagocytosis. Aside from these three inhibitors (tirofiban, podoplanin blocking mabs, wortmannin) other inhibitors/blocking mabs were tested too, but they didn't show reduction in comparison to the control. Table 7 below shows the different inhibitors/blocking mabs tested for change in comparison to control.

TABLE 7

| Inhibitor | Affected mechanism |
|---|---|
| Significance | |
| Podoplanin | the interaction between Podoplanin (cancer cell) |
| blocking mabs | and CLEC-2 (platelets) |
| Tirofiban | Inhibitor of GP IIb/IIIa receptors |
| Wortmannin | Inhibitor of receptor-mediated phagocytosis |
| No Significance | |
| P-selectin | the interaction between P-selectin (platelets) |
| blocking mabs | and PSGL-1 (cancer cell) |
| Chlorpromazine | Inhibitor of clathrin-mediated endocytosis |
| Genistein | Inhibitor of caveolae-mediated endocytosis |

### Example 9: In Vitro Cytotoxicity of DOX-CPP, DOX-FPH, Doxosome, and Free DOX Against HepG2 Cells Determined by WST-1 Assay

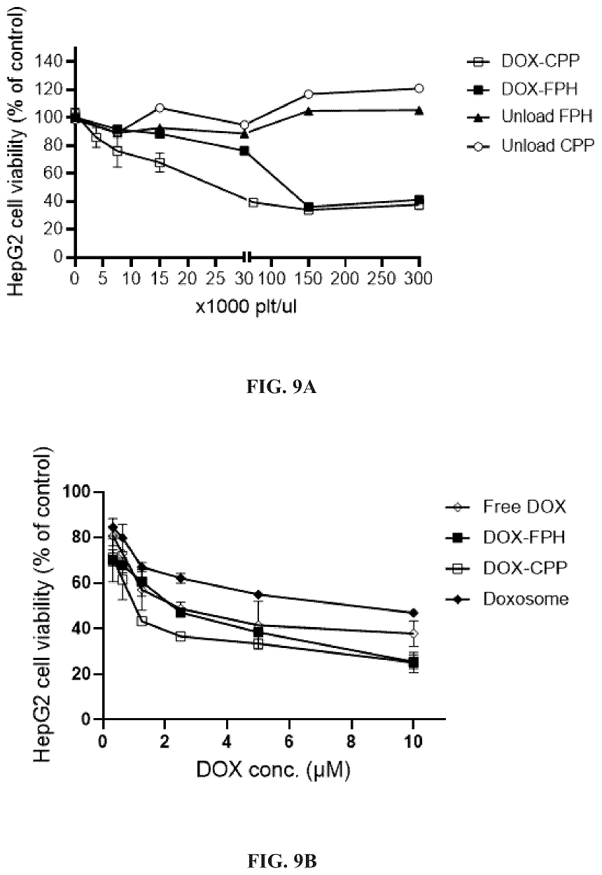

Dox-CPP, and DOX-FPH were prepared by the method of Example 1. WST-1 assay is water-soluble tetrazolium salt assay that provides a mechanism to measure cell viability and cytotoxicity in cells. An increase in the number of viable cells leads to an increase in the activity of mitrochondrial dehydrogenases which in turn results in an increase in the amount of formazan dye produced. The formazan dye produced from WST-1 by viable cells is quantified by colorimetric measurement of the absorbance pf the dye at OD-44-nm. HepG2 cells were seeded on 96 well plates at a density of 5×10³ per well. After 24 hours of incubation, DOX-CPP, DOX-FPH, Doxosome, and Free-DOX were mixed into fresh 0.5% FBS DMEM (ATCC-formulated Eagle's Minimum Essential Medium, catalog No. 30-2003) culture medium with equivalent DOX concentrations ranging from 0, 0.25, 2.5, 5, 10, and 20 μM. All DOX solutions were then added to the 96 well plates. Unloaded CPP and unloaded FPH were used as controls. After treatment for 48 hours, the cultured medium was removed and fresh 0.5% FBS DMEM medium containing WST-1 dye (90 μl medium plus 10 μl WST-1 reagent) was added to each well and incubated again for 2 hours at 37° C. The absorbance was measured at 440 nm using microplate reader and cell viability was expressed as a percentage of the untreated cells. All treatment groups had two replicates. FIG. 9A shows that with the same platelet counts, DOX-CPP has increased HepG2 inhibition relative to DOX-FPH. Also unloaded CPP and unloaded FPH did not affect the cancer cell growth. FIG. 9B shows the cell viability is decreased with increasing doxorubicin concentration. Furthermore IC₅₀ (table below) for HepG2 is reduced by 3-3.5 fold in DOX-CPP and DOX-FPH relative to Doxosomes in 48 hours of treatment.

TABLE 8

| DOX | IC₅₀ (μM) |
|---|---|
| DOX-CPP | 2.0 |
| DOX- FPH | 2.9 |
| Free-DOX | 4.3 |
| Doxosome | 6.8 |

### Example 10: DIR Labeling of DOX-CPP and Doxosomes and Distinguishing Between DiR Signal and Doxorubicin Signal

DOX-CPP was prepared according to Example 1. Both DOX-CPP and Doxosomes were labeled with DiR dye. DiR dye is lipophilic carbocyanine with near IR absorption and emission. DIR dyes insert into the phospholipid bilayer membrane, staining the entire cell surface. To label DOX-CPP and Doxosomes with DiR dye, first CBC analysis was performed followed by adjustment with PBS of each product. 50 μM of DiR dye was then incubated in each product (DOX-CPP and Doxosomes) for 30 minutes at room temperature. For DOX-CPP labeled product, the sample was spun at 1500 g for 20 minutes and then washed with PBS before analysis. The Doxosome labeled product was used DiRectly for analysis. The DiR DOX-CPP sample was captured on the plate reader to confirm no fluorescence spectra overlap between DiR and doxorubicin (spectra not shown). It was observed that the doxorubicin signal is at 480/585 nm and the DiR signal is at 748/780 nm, the plate reader shows no interference of the two signals. DiR labeled Doxosome and DiR labeled DOX-CPP was done to enable visualization using IVIS® (In Vivo Imaging System) Lumina™ S5 Imaging System (Perkin Elmer, perkinelmer.com) for pilot animal study.

### Example 11: Phase 0: Perform Time Course and Assess Biodistribution of DiR Labeled DOX-CPP and DiR Labeled Doxosome in B-NGD Mice (Non-Tumor) for Future Tumor Bearing B-NDG Mice Study

The optimized time course for DIR labeled DOX-CPP and DIR labeled Doxosomes was evaluated based on fluorescence signal changes detected by IVIS. The animals' care and housing were in accordance to standard, Biocytogen Boston (biocytogen.com Boston, MA) Institutional Animal Care and Use Committee. An acclimation period of approximately a week was implemented between animal receipt and any experimental procedures. The products that were tested for time course and biodistribution were DIR labeled DOX-CPP and DIR labeled Doxosomes.

To create the DIR labeled DOX-CPP and DiR labeled Doxosomes, the following steps were performed at Biocytogen. 5 mg/mL of DiR stock solution was created by adding 2 mL of DMSO into solid DiR bottle (10 mg in bottle). The DiR stock solution was stored at room temperature in the absence of light. DOX-CPP arrived at Biocytogen stored at −80° C. The DOX-CPP vial was thawed in a 37° C. water bath for approximately 2-3 minutes and used within 3 hours post thaw. Doxosome solution was created by adding 50 μl of Doxosomes into 1 ml of PBS. 5 mg/mL of DiR stock solution was added to thawed DOX-CPP product, the combination was mixed well by gently pipetting about 10 minutes. The DiR and DOX-CPP combination was incubated on the rocker for 30 minutes in the dark followed by centrifugation at 1400 rpm for 15 minutes at room temperature. The supernatant was then removed and the cells were resuspended in 1 ml PBS at room temperature. Finally, the solution was mixed by gently pipetting for about 20 minutes. The DiR labeled DOX-CPP was used within 2 hours after labeling and gently pipetted again before injection. For DiR labeled Doxosome 5 mg/mL of DiR solution was added to Doxosome solution to achieve a final DiR dye concentration of approximately 50 μg/ml and mixed well by gently pipetting. The DiR and Doxosome solution were incubated on the rocker for 30 minutes in the dark. The DiR labeled Doxosomes were directly used and again gently pipetted before injection.

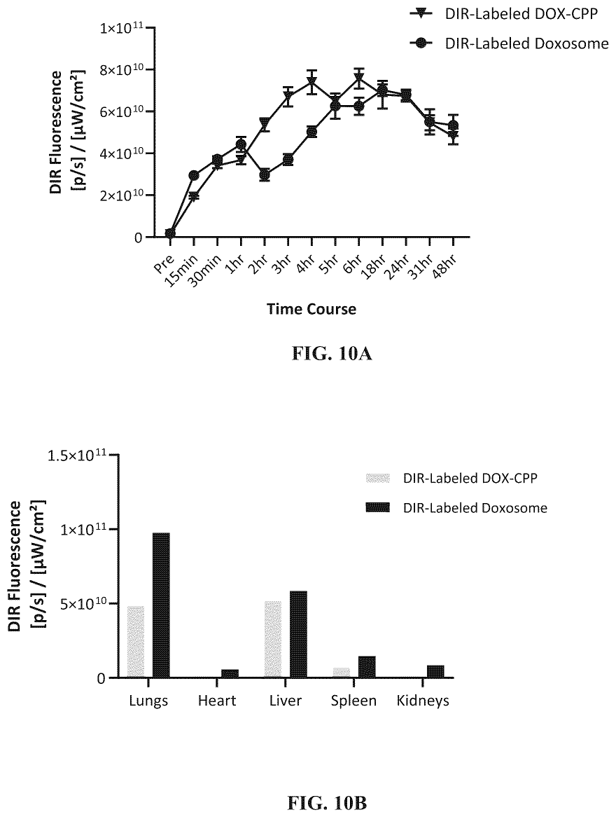

Six non-tumor B-NDG mice were separated in two groups (n=3) for administration of the two products (DiR labeled DOX-CPP and DiR labeled Doxosomes). Pre-IVIS image of each mouse was taken prior to administration of the products. 200 μl volume of DiR labeled DOX-CPP or DiR labeled Doxosomes was administered to each mouse tail vein injection. There were about 1.6×10⁹ platelets/mL of Dir labeled DOX-CPP and 0.1 mg/ml of DiR labeled Doxosomes. In vivo IVIS imaging was done at different time courses (15 min, 30 min, 1 h, 2 h, 3 h, 4 h, 6 h, 18 h, 24 h, 48 h after dosing) for DIR labeled DOX-CPP and DIR labeled Doxosomes. Prior to each image being taken, isoflurane anesthesia was delivered to the mouse. FIG. 10A shows the distribution of DiR labeled DOX-CPP and DiR labeled Doxosomes during the different time courses. Based on the time course study, 6 hours was chosen as the starting time for imaging for Phase 1a study. Sustained retention was observed up to 48 hours post administration. FIG. 10B shows the accumulation of the two products in different organs. Both products preferentially accumulate in the liver as shown. Accumulation of DiR labeled Doxosomes is also prominent in the lungs. Furthermore, DiR labeled Doxosomes appear to spread to multiple organs, for example, heart and kidneys, when compared to DiR labeled DOX-CPP.

### Example 12: Phase 1a: Evaluate the Accumulation of Dir Labeled DOX-CPP and DiR Labeled Doxosome into Hep3B Luc Orthotopic Human Liver Tumor in B-NDG Mice

The Hep3B-Luc tumor cell line was maintained in vitro as a monolayer culture in EMEM supplement with 2 mM L-Glutamine and 10% fetal bovine serum (FBS) in a humidified incubator at 37° C. in an atmosphere with 5% CO2. The cells grew in an exponential growth phase and were harvested for inoculation. Mice were acclimated for 2 weeks prior to inoculation intra-hepatically with Hep3B-Luc tumor cells, therefore, all the mice studied were orthotopic models. Mice were anesthetized using 2% isoflurane in an anesthesia induction chamber. Mice were inoculated intra-hepatically with 0.5×10⁶ Hep3B-Luc per mouse. Mice bearing HepBB-Luc orthotopic liver tumor were enrolled into groups 8-10 days post inoculation based on tumor load as determined by bioluminescence imaging (BLI) using the IVIS. There were 6 groups of tumor bearing mice, 5 in each group. DiR labeled DOX-CPP and DiR labeled Doxosomes were prepared according to the process described in Example 11. Dir-labeled DOX-CPP and DiR labeled Doxosomes were prepared in three different doses, undiluted, 1:4 diluted, and 1:10 diluted. The products were diluted with PBS. Non-diluted was having a dose of 2.18 mg/kg, 1:4 diluted was having a dose of 0.54 mg/kg, and 1:10 diluted was having a dose of 0.218 mg/kg. Each group consisted of 5 mice and were dosed according to Table 9 below. 200 μl volume of DiR labeled DOX-CPP or DiR labeled Doxosomes at the different doses was administered intravenously per mouse by tail vein injection. The mice used for this study were administered with either Dir-labeled DOX-CPP or DiR labeled Doxosomes once and the day of the administration was considered as day 0. Mice were maintained on 2.5% isoflurane via nose cones.

TABLE 9

| | Treatment | | | | Route of | Imaging for DIR |
|---|---|---|---|---|---|---|
| G | Groups | N | Dose | | Administration | 748/780 nm Signal |
| 1 | DIR labeled DOX- | 5 | 2.18 | mg/kg | IV | Pre-, 6 h, 12 h, 24 h, 48 h, |
| | CPP, non-diluted | | | | | and 24 days |
| 2 | DIR labeled DOX- | 5 | 0.54 | mg/kg | IV | Pre-, 6 h, 12 h, 24 h, 48 h, |
| | CPP, 1:4 diluted | | | | | and 24 days |
| 3 | DIR labeled DOX- | 5 | 0.218 | mg/kg | IV | Pre-, 6 h, 12 h, 24 h, 48 h, |
| | CPP, 1:10 diluted | | | | | and 24 days |
| 4 | DIR labeled | 5 | 2.18 | mg/kg | IV | Pre-, 6 h, 12 h, 24 h, 48 h, |
| | Doxosomes, non- | | | | | and 24 days |
| | diluted. | | | | | |
| 5 | DIR labeled | 5 | 0.54 | mg/kg | IV | Pre-, 6 h, 12 h, 24 h, 48 h, |
| | Doxosomes, 1:4 | | | | | and 24 days |
| | diluted | | | | | |
| 6 | DIR labeled | 5 | 0.218 | mg/kg | IV | Pre-, 6 h, 12 h, 24 h, 48 h, |
| | Doxosomes 1:10 | | | | | and 24 days |
| | diluted | | | | | |

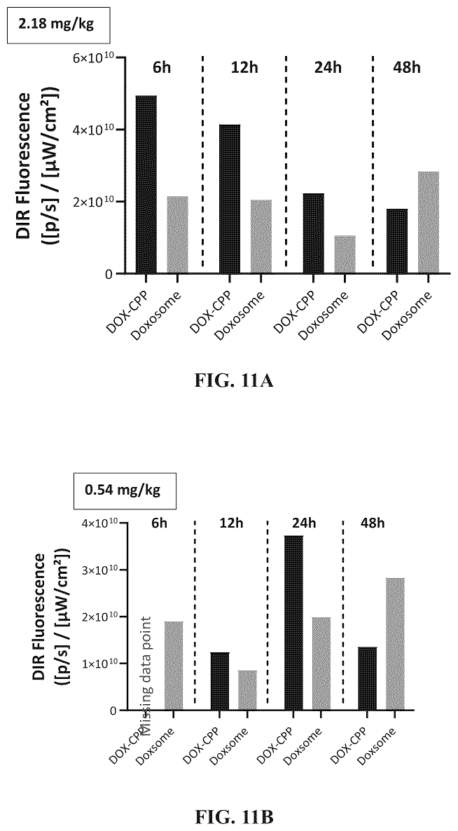

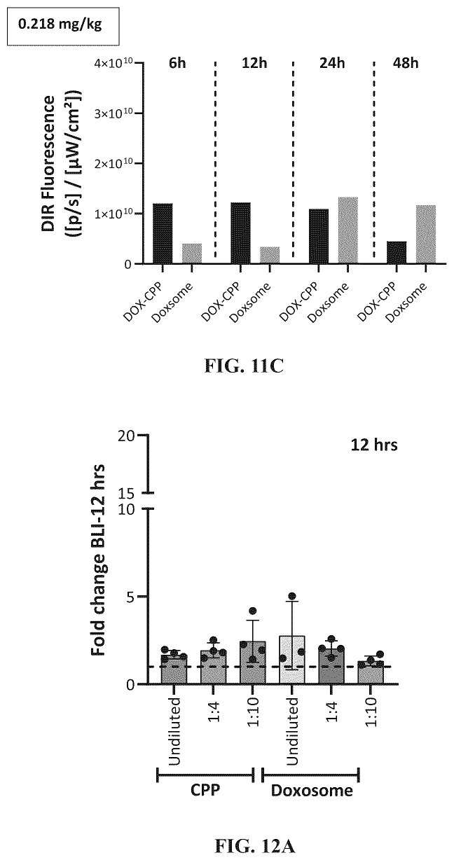

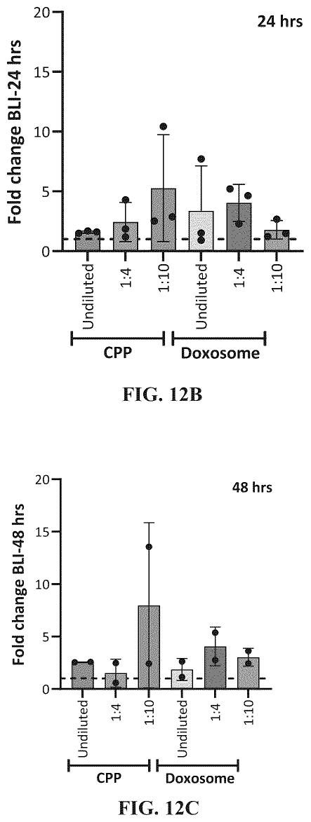

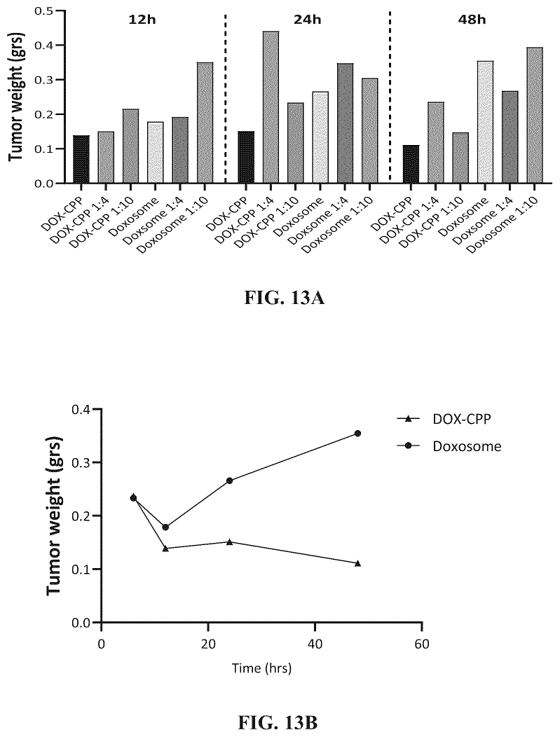

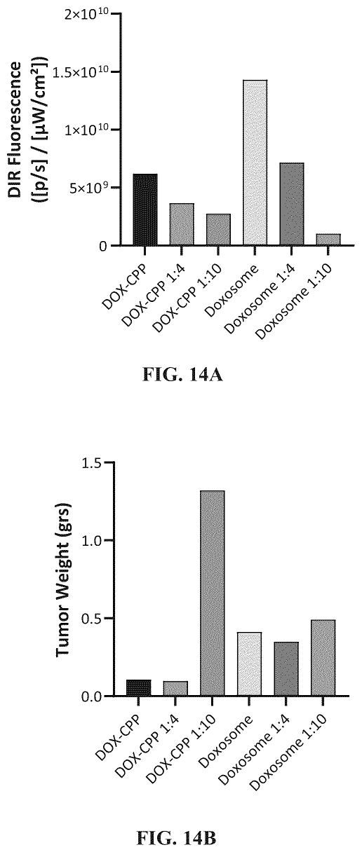

At each time point after dosing of the product an in-vivo IVIS image was captured for all mice. Following this, one mouse was selected from each group to be sacrificed to harvest the organs for ex-vivo IVIS imaging of the organs and ex-vivo weight of the tumor. FIG. 11A-11C show the DOX-CPP and Doxosomes accumulation for the different dosages for time points, 6 h, 12 h, 24 h, and 48 h. In vivo bioluminescence imaging (BLI) using IVIS was used to measure the tumor burden of the different doses at the various timepoints. Due to the wide distribution of the tumor burden measurements pre-treatment, a fold change was calculated to normalize the data. A fold change is the tumor burden measurement at a specific timepoint/pre-treatment signal. FIG. 12A-12C show the tumor burden fold change of the different doses at three different time points. FIG. 12A-12C show an overall decrease in tumor burden (BLI) for samples treated with DOX-CPP in a dose dependent manner. There is no clear dose correlation observed for the Doxosomes treated mice. FIG. 13A shows the tumor weight for all three doses of each product at different time points. FIG. 13B shows the tumor weight as a function of time for the undiluted dose (2.18 mg/kg) for each product. Overall tumor burden as measured by tumor weight in FIG. 13A decreased for mice treated with DOX-CPP compared to Doxosomes treated mice, more specifically the decrease in tumor weight is evident for the undiluted dose of DOX-CPP in comparison Doxosomes as shown in FIG. 13B. FIG. 14A shows the product accumulation of the three dosages at the tumor site at 24 days; FIG. 14B shows the tumor weight at 24 days from the three dosages of the two products. It can be observed that undiluted DOX-CPP, and DOX-CPP 1:4 is able to effectively reduce the weight of tumor as compared to undiluted Doxosomes, and Doxosomes 1:4 (FIG. 14B), despite less product accumulation (FIG. 14A) at 24 days.

### Example 13. One Year Stability Study of Pooled CPP Product Stored at −20° C. (Transition Temperature Cryopreserved-Product)

The stability of a pooled CPP product was tested at different timepoints for a storage period of 1 year. 7 Apheresis Platelet Units (APU) were pooled resulting in 7 units or cryo-vessels of Pooled CPP product. Initially the 7 units (having unloaded platelets) were stored at ≤−65° C. freezer for a minimum of 24 hours to form an initial frozen platelet composition. After the initial storage, the cryo-vessels were transferred to a −20° C. freezer for storage to form cryopreserved platelets (transition temperature cryopreserved-product). One unit of the Pooled CPP product (transition temperature cryopreserved-product) was removed at different time points and tested according to the criteria below.
Visual Inspection of cracks, tears, breaks of the unit bag.
Visual Inspection of aggregate free swirling of the product in the bag.
Platelet count per bag.
pH of the product.

Before initiating each test, the product was removed from the freezer, thawed in a 37° C. water bath for 8 minutes, then rehydrated by adding 25 mL of 0.9% saline. Visual inspection of breaks, visual inspection of aggregate free swirling, platelet count per bag, and pH of product are indicators for the release criteria of the product. The platelet count per bag was obtained using either the Beckman Coulter AcT Diff 2 Hematology Particle Analyzer or the Beckman Coulter D×H Hematology Analyzer (Beckman Coulter, beckmancoulter.com). The table below (Table 10) details the results of the tests time points.

### Platelet Count

The platelet count per bag was obtained using either the Beckman Coulter AcT Diff 2 Hematology Particle Analyzer or the Beckman Coulter D×H Hematology Analyzer (Beckman Coulter, beckmancoulter.com).

TABLE 10

Release Criteria

| | | Aggregate- | | Platelet |
|---|---|---|---|---|
| | Visual | free | | Count/Bag |
| Timepoint | Inspection | Swirling | pH | (≥1.7 × |
| (Months) | (Pass/Fail) | (Pass/Fail) | (≥6.2) | 10¹¹) |
| 1 | Pass | Pass | 7.3 | 2.20E+11 |
| 3 | Pass | Pass | 7.6 | 2.20E+11 |
| 6 | Pass | Pass | 7.7 | 2.20E+11 |
| 9 | Pass | Pass | 7.8 | 2.10E+11 |
| 12 | Pass | Pass | 7.7 | 2.10E+11 |

The results of the stabilization study demonstrate product stability of a transition temperature cryopreserved-product after 12 months of storage at −20° C. The product at all time points passed all release criteria. There were no cracks, breaks, or leaks by visual inspection. Aggregate-free swirl test was confirmed at all time points. The minimum pH and minimum platelet count/bag criteria were achieved at all time points. Although a gradual increase in pH was observed for the 12 months, it did not affect the viability of the product. The platelet count/bag was consistent at the different time points. The findings here confirm the stability of the CPP pooled product (transition temperature cryopreserved-product) at the −20° C. storage temperature for 1 year. Therefore, the present Example provides a proof-of-concept that cryopreserved platelets obtained as per a process comprising a transition in a freezing temperature and a storing temperature is capable of being stored at −20° C. for at least 1 year.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

While the embodiments of the invention are amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

What is claimed is:

1. A process for preparing a cryopreserved anti-cancer drug-loaded platelet composition comprising cryopreserved anti-cancer drug-loaded platelets, said process comprising:
a) incubating platelets with an anti-cancer drug in the presence of a loading buffer comprising a monosaccharide and/or a disaccharide at a temperature in the range of 18-42° C. for a time period in the range of 20 minutes to 12 hours, to obtain a population of anti-cancer drug-loaded platelets;
b) freezing the population of anti-cancer drug-loaded platelets in a cryopreservation medium at a temperature of equal to or less than −50° C. to form an initial frozen anti-cancer drug-loaded platelet composition; and
c) storing the initial frozen anti-cancer drug-loaded platelet composition at a temperature in the range of −10° C. to −30° C. for at least 10 days to form the cryopreserved anti-cancer drug-loaded platelet composition.

2. The process of claim 1, wherein the anti-cancer drug is a chemotherapy drug, and wherein the chemotherapy drug is selected from an alkylating agent, an antimetabolite, an anthracycline, an antitumor antibiotic, a platinum-containing compound, or a plant alkaloid.

3. The process of claim 1, wherein the anti-cancer drug is a chemotherapy drug.

4. The process of claim 1, wherein the anti-cancer drug is doxorubicin, epirubicin, daunorubicin, idarubicin, or valrubicin.

5. The process of claim 1, wherein the anti-cancer drug is doxorubicin.

6. The process of claim 1, wherein the storing comprises storing the initial frozen anti-cancer drug-loaded platelet composition in a freezer set at a temperature of −20° C.+/−2° C.

7. The process of claim 1, wherein the freezing comprises subjecting the population of anti-cancer drug-loaded platelets in the cryopreservation medium at the temperature for at least 30 minutes to form the initial frozen anti-cancer drug-loaded platelet composition.

8. The process of claim 1, wherein the process further comprises thawing the anti-cancer drug-loaded cryopreserved platelets to form a liquid platelet composition comprising anti-cancer drug-loaded platelets, and administering an effective amount of the liquid platelet composition comprising the anti-cancer drug-loaded platelets to a subject in need thereof.

9. The process of claim 1, wherein the storing is done for a time period in the range of 1 month to 1 year.

10. The process of claim 1, wherein the cryopreservation medium comprises dimethyl sulfoxide (DMSO) in a concentration in the range of 5% to 8%.

11. The process of claim 1, wherein the cryopreservation medium comprises 0.5-5.5% DMSO, 0.5-3% glycerol, and 3-10% polysucrose.

12. The process of claim 1, wherein the initial frozen anti-cancer drug-loaded platelet composition is stored at the temperature of in the range of −10° C. to −30° C. for at least 6 months.

13. The process of claim 1, wherein a therapeutically effective amount of the cryopreserved anti-cancer drug-loaded platelets upon thawing retain the ability to kill cancer cells.

14. A composition comprising frozen anti-cancer drug-loaded platelets in a cryopreservation medium in a frozen state, wherein the composition is capable of yielding the following recited properties after storage for 6 months, upon thawing:
a) is in a liquid state without requiring the addition of a liquid to achieve such liquid state;
b) exhibits a platelet count of at least 1.0×10^{11/35} ml of the composition;
c) yields a single peak that corresponds to a compromised membrane peak in a membrane integrity assay;
d) exhibits a CD61-positive-microparticle content of less than 50% of the CD61 positive particles in the composition; and
e) retains at least 30%, 35%, 40%, 45%, 50%, 60%, or 70% of the anti-cancer drug.

15. The composition of claim 14, wherein the membrane integrity assay comprises incubating the composition with calcein acetoxymethyl (AM) to form a treated composition and analyzing the treated composition by flow cytometry.

16. The composition of claim 14, wherein the frozen anti-cancer drug-loaded platelets upon thawing have a diameter in the range of 0.5-5 μm and wherein the composition upon thawing has a CD61-positive-microparticle content in the range of 10-30%.

17. The composition of claim 14, wherein the anti-cancer drug is a chemotherapy drug, and wherein the chemotherapy drug is selected from an alkylating agent, an antimetabolite, an anthracycline, an antitumor antibiotic, a platinum-containing compound, or a plant alkaloid.

18. The composition of claim 14, wherein the anti-cancer drug is a chemotherapy drug.

19. The composition of claim 14, wherein the anti-cancer drug is doxorubicin, epirubicin, daunorubicin, idarubicin, or valrubicin.

20. The composition of claim 14, wherein the anti-cancer drug is doxorubicin.
